SEQUENCE LISTING (64 sequences)

SEQ ID NO: 1 - protein, 934 aa, Homo sapiens
  misc_feature: Incyte ID No 2072638CD1
MTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 2 - protein, 236 aa, Homo sapiens
  misc_feature: Incyte ID No 747515CD1
MASLDRVKVLVLGDSGVGKSSLVHLLCQNQVLGNPSWTVGCSVDVRVHDYKEGTPEEKTYYIELWDVGGSVGSASSVKSTRAVFYNSVNGIIFVHDLTNKKSSQNLRRWSLEALNRDLVPTGVLVTNGDYDQEQFADNQIPLLVIGTKLDQIHETKRHEVLTRTAFLAEDFNPEEINLDCTNPRYLAAGSSNAVKLSRFFDKVIEKRYFLREGNQIPGFPDRKRFGAGTLKSLHYD

SEQ ID NO: 3 - protein, 171 aa, Homo sapiens [sequence omitted]

SEQ ID NO: 4 - protein, 316 aa, Homo sapiens [sequence omitted]

SEQ ID NO: 5 - protein, 513 aa, Homo sapiens [sequence omitted]

SEQ ID NO: 6 - protein, 123 aa, Homo sapiens
  misc_feature: Incyte ID No 948883CD1
MLCWSAFAMGIGQVRAPPKSPRGCCKAFCSSFCASAAISSTVRGSSKPKCVITVARVCAAGCTWAYAESLWGVRRLLLNPNETLSGVTGLPYVRVSMPRSCSARLRRFCWSASCVRKVAGAGV

SEQ ID NO: 7 - protein, 125 aa, Homo sapiens
  misc_feature: Incyte ID No 5665403CD1
MILYTIFLKVQEDSADYGDLWASLRIAFPLRFFLSVSHTFSPNFRHTLPAFPLLIHSSPSLNLIVYPVACCLCTEGRWMESVLSCPCWPSWCLPSAQSLHSPCCSYFSAMHSFRRSVINFFLIPL

SEQ ID NO: 8 - protein, 267 aa, Homo sapiens
  misc_feature: Incyte ID No 7493065CD1
MRVAALISGGKDSCYNMMQCIAAGHQIVALANLRPAENQVGSDELDSYMYQTVGHHAIDLYAEAMALPLYRRTIRGRSLDTRQVYTKCEGDEVEDLYELLKLVKEKEEVEGISVGAILSDYQRIRVENVCKRLNLQPLAYLWQRNQEDLLREMISSNIQAMIIKVAALGLDPDKHLGKTLDQMEPYLIELSKKYGVHVCGEGGEYETFTLDCPLFKKKIIVDSSEVVIHSADAFAPVAYLRFLELHLEDKVSSVPDNYRTSNYIYNF

SEQ ID NO: 9 - protein, 71 aa, Homo sapiens
  misc_feature: Incyte ID No 7493531CD1
MAICGSALHFHDSLYHGTTGILYMSAAVQPALLSFPCGMAPSHGRELIVTHSSFEVHFHAANTVHADIPKT

SEQ ID NO: 10 - protein, 642 aa, Homo sapiens
  misc_feature: Incyte ID No 3321454CD1
MAAEWGGGVGYSGSGPGRSRWRWSGSVWVRSVLLLLGGLRASATSTPVSLGSSPPCRHHVPSDTEVINKVHLKANHVVKRDVDEHLRIKTVYDKSVEELLPEKKNLVKNKLFPQAISYLEKTFQVRRPAGTILLSRQCATNQYLRKENDPHRYCTGECAAHTKCGPVIVPEEHLQQCRVYRGGKWPHGAVGVPDQEGISDADFVLYVGALATERCSHENIISYAAYCQQEANMDRPIAGYANLCPNMISTQPQEFVGMLSTVKHEGFSAGLFAFYHDKDGNPLTSRFADGLPPFNYSLGLYQWSDKVVRKVERLWDVRDNKIVRHTVYLLVTPRVVEEARKHFDCPVLEGMELENQGGVGTELNHWEKRLLENEAMTGSHTQNRVLSRITLALMEDTGRQMLSPYCDTLRSNPLQLTCRQDQRAVAVCNLQKFPKPLPQEYQYFDELSGIPAEDLPYYGGSVEIADYCPFSQEFSWHLSGEYQRSSDCRILENQPEIFKNYGAEKYGPHSVCLIQKSAFVMEKCERKLSYPDWGSGCYQVSCSPQGLKVWVQDTSYLCSRAGQVLPVSIQMNGWIHDGNLLCPSCWDFCELCPPETDPPATNLTRALPLDLCSCSSSLVVTLWLLLGNLFPLLAGFLLCIWH

SEQ ID NO: 11 - protein, 277 aa, Homo sapiens
  misc_feature: Incyte ID No 189299CD1
MGNTIRALVAFIPADRCQNYVVRDLREMPLDKMVDLSGSQLRRFPLHVCSFRELVKLYLSDNHLNSLPPELGQLQNLQILALDFNNFKALPQVVCTLKQLCILYLGNNKLCDLPSELSLLQNLRTLWIEANCLTQLPDVVCELSLLKTLHAGSNALRLLPGQLRRLQELRTIWLSGNRLTDFPTVLLHMPFLEVIDVDWNSIRYFPSLAHLSSLKLVIYDHNPCRNAPKVAKGVRRVGRWAEETPEPDPRKARRYALVREESQELQAPVPLLPPTNS

SEQ ID NO: 12 - protein, 419 aa, Homo sapiens
  misc_feature: Incyte ID No 7488057CD1
MGPLSAPPCTQHITWKGLLLTASLLNFWNPPTTAQVTIEAQPPKVSEGKDVLLLVHNLPQNLTGYIWYKGQIRDLYHYVTSYVVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGENLYLSCFADSNPPAQYSWTINGKFQLSGQKLSIPHITTKHSGLYACSVRNSATGKESSKSMTVKVSDWTLP

SEQ ID NO: 13 - protein, 142 aa, Homo sapiens
  misc_feature: Incyte ID No 7486411CD1
MSFLTVPYKLPVSLSVGSCVIIKGTLIDSSSNEPQLQVDFYTEMNEDSEIAFHLRVHLGRRVVMNSREFGIWMLEENLHYVPFEDGKPFDLRIYVCHNEYEVKVNGEYIYAFVHRIPPSYVKMIQVWRDVSLDSVLVNNGRR

SEQ ID NO: 14 - protein, 119 aa, Homo sapiens [sequence omitted]

SEQ ID NO: 15 - protein, 249 aa, Homo sapiens [sequence omitted]

SEQ ID NO: 16 - protein, 314 aa, Homo sapiens [sequence omitted]

SEQ ID NO: 17 - protein, 183 aa, Homo sapiens [sequence omitted]

SEQ ID NO: 18 - protein, 621 aa, Homo sapiens [sequence omitted]

SEQ ID NO: 19 - protein, 79 aa, Homo sapiens [sequence omitted]

SEQ ID NO: 20 - protein, 83 aa, Homo sapiens [sequence omitted]

SEQ ID NO: 21 - protein, 204 aa, Homo sapiens
  misc_feature: Incyte ID No 2771521CD1
MLVAAAAERNKDPILHVLRQYLDPAQRGVRVLEVASGSGQHAAHFARAFPLAEWQPSDVDQRCLDSIAATTQAQGLTNVKAPLHLDVTWGWEHWGGILPQSLDLLLCINMAHVSPLRCTEGLFRAAGHLLKPRALLITYGPYAINGKISPQSNVDFDLMLRCRNPEWGLRDTALLEDLGKASGLLLERMVDMPANNKCLIFRKN

SEQ ID NO: 22 - protein, 83 aa, Homo sapiens
  misc_feature: Incyte ID No 7095792CD1
MIVFIAIFLMLVSSSQSCLSFACCVYLLSFNFEQFLSLSLVSFAIDSFEEASYFVECPSFWVCLLFSHDHMRVRHFWQAYYQK

SEQ ID NO: 23 - protein, 174 aa, Homo sapiens
  misc_feature: Incyte ID No 7112696CD1
MNSISAVNAFRFLALATVAAAMTGCSFLQVGESEYSCKGMPDGVTCMSARDVYQLTENENFRQVVEQNQSAKDQAIKEGKSIEEVLPAVQPHIAAGERYVVPKPARNPIPIRSQATVMRVWVAPWESDSGDLNVPGFIYTEIEPRRWEIGTPAPKPTPSIRPLESRKNTSSPAT

SEQ ID NO: 24 - protein, 771 aa, Homo sapiens
  misc_feature: Incyte ID No 7759388CD1
MAPGPFSSALLSPPPAALPFLLLLWAGASRGQPCPGRCICQNVAPTLTMLCAKTGLLFVPPAIDRRVVELRLTDNFIAAVRRRDFANMTSLVHLTLSRNTIGQVAAGAFADLRALRALHLDSNRLAEVRGDQLRGLGNLRHLILGNNQIRRVESAAFDAFLSTVEDLDLSYNNLEALPWEAVGQMVNLNTLTLDHNLIDHIAEGTFVQLHKLVRLDMTSNRLHKLPPDGLFLRSQGTGPKPPTPLTVSFGGNPLHCNCELLWLRRLTREDDLETCATPEHLTDRYFWSIPEEEFLCEPPLITRQAGGRALVVEGQAVSLRCRAVGDPEPVVHWVAPDGRLLGNSSRTRVRGDGTLDVTITTLRDSGTFTCIASNAAGEATAPVEVCVVPLPLMAPPPAAPPPLTEPGSSDIATPGRPGANDSAAERRLVAAELTSNSVLIRWPAQRPVPGIRMYQVQYNSSVDDSLVYRMIPSTSQTFLVNDLAAGRAYDLCVLAVYDDGATALPATRVVGCVQFTTAGDPAPCRPLRAHFLGGTMIIAIGGVIVASVLVFIVLLMIRYKVYGDGDSRRVKGSRSLPRVSHVCSQTNGAGTGAAQAPALPAQDHYEALREVESQAAPAVAVEAKAMEAETASAEPEVVLGRSLGGSATSLCLLPSEETSGEESRAAVGPRRSRSGALEPPTSAPPTLALVPGGAAARPRPQQRYSFDGDYGALFQSHSYPRRARRTKRHRSTPHLDGAGGGAAGEDGDLGLGSARACLAFTSTEWMLESTV

SEQ ID NO: 25 - protein, 841 aa, Homo sapiens
  misc_feature: Incyte ID No 8165414CD1
MSVVGIDLGFQSCYVAVARAGGIETIVNEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGHGDNPGPRLLSRGTEHSCDFGFSTRKLPEMDID

SEQ ID NO: 26 - protein, 394 aa, Homo sapiens
  misc_feature: Incyte ID No 2540610CD1
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHRPRILEMDKEENRRSVLLPTHRRRGSFSSENYWRKSYESSEDCSEAAGSPARKVKMRRH

SEQ ID NO: 27 - protein, 196 aa, Homo sapiens [sequence omitted]

SEQ ID NO: 28 - protein, 87 aa, Homo sapiens
  misc_feature: Incyte ID No 1480069CD1
MLSCLLTSFSMAPVEGTVRWQQCRVVERPRGRRFEADQLLSVKQYSGRSTDFGVTQTWVLVPAYLYQLCDLGEVAEPLCASVSICTK

SEQ ID NO: 29 - protein, 233 aa, Homo sapiens
  misc_feature: Incyte ID No 2310442CD1
MAWTVLLLGLLSHCTGYVTSYVLTQPPSVSVAPGKTARISCGGNNIGSKSVHWYQQKPGQAPVLVVYDDSDRPSGIPERFSGSNSGNTATLTISRVEAGDEADYYCQVWDSSSDHSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 373 aa, Homo sapiens
  misc_feature: Incyte ID No 7503731CD1
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSRGGGAEQPSFKWGCIRLGLFKSNKMFWLRKLFCFQCKSSQCSKQSSVFCSPLSLTDVCTWLRSPGASQALLFSTSHLPSTPCKLMQTPFLPPAAELFRLPGQGLKQCQPLPSQSYC

SEQ ID NO: 31 - protein, 301 aa, Homo sapiens
  misc_feature: Incyte ID No 7506368CD1
MALAARLWRLLPFRRGAAPGSRLPAGTSGSRGHCGPCRFRGFEVMGNPGTFNRGLLLSALSYLGFETYQVISQAAVVHATAKVEEILEQADYLYESGETEKLYQLLTQYKESEDAELLWRLARASRDVAQLSRTSEEEKKLLVYEALEYAKRALEKNESSFASHKWYAICLSDVGDYEGIKAKIANAYIIKEHFEKAIELNPKDATSIHLMGIWCYTFAEMPWYQRRIAKMLFATPPSSTYEKALGYFHRAEQGKTYLKLHNKKLAAFWLMKAKDYPAHTEEDKQIQTEAAQLLTSFSEKN

SEQ ID NO: 32 - protein, 193 aa, Homo sapiens
  misc_feature: Incyte ID No 7509087CD1
MLVAAAAERNKDPILHVLRQYLDPAQRGVRVLEVASGSGQHAAHFARAFPLAEWQPSDVDQRCLDSIAATTQAQGLTNVKAPLHLDVTWGWEHWGGILPQSLDLLLCINMAHVSPLRCTEGLFRAAGHLLKPRALLITYGGGGALSFSKPQPASLSQALWEIPRPWSSGLCPHAHSPMPSMGRSPPRATWTLT

SEQ ID NO: 33 - DNA, 3716 bp, Homo sapiens
  misc_feature: Incyte ID No 2072638CB1
ccatcttccacctgcagccttctgaccaggcgaattctactgcgaggccgccgagtggatccaggatccggatgggtcgtggtatgctatgacccgaaagcgttccgagggagccgtggtcaacgtccagccaactgacaaagaattcactgttcggctggagacagagaagcggctgcacacggtgggcgagccggtggagttcagatgcatcctggaggctcagaatgttcccgaccgttactttgctgtctcctgggccttcaacagctcgctcatcgccaccatgggtcctaacgctgtgcctgtcctcaacagcgaatttgctcaccgggaagccaggggacagcttaaggtggccaaagagagcgacagtgtctttgtgctgaagatctaccacctccgccaggaagatagcgggaaatacaactgccgggtgactgagcgagagaaaaccgtgaccggggaattcattgataaggagagcaagcgtcccaagaacatccccatcatagtcctccccctcaagagcagcatctccgtggaggtggccagcaatgccagcgtcatccttgagggcgaggacctgcgcttctcctgcagtgtccgcacggcaggcaggccgcagggtcgcttctctgtcatctggcagcttgtggacaggcagaaccgccgcagcaatatcatgtggctagaccgggatggcaccgtgcagccaggctcgtcctactgggagcgcagcagctttgggggcgtccagatggagcaggtgcagcccaactcgttcagcctgggcatcttcaacagcaggaaggaggacgagggccagtatgaatgccatgtgactgaatgggtgcgggcagtggatggcgagtggcagattgttggggagcgccgggccagcactcccatctccatcacagctcttgaaatgggcttcgcagtcacagccatctcccggacaccgggggtgacctacagcgactcctttgacttgcagtgtatcatcaaaccccactaccctgcctgggtccccgtgtcggtgacatggcggttccagccggtgggcacggtggagttccatgacttggtgaccttcacccgggacggaggggtccagtggggggacaggtcctccagcttccgaacccgaactgccatcgagaaggctgagtccagcaacaacgtccgcctaagcatcagccgagccagtgacacggaagcaggcaagtaccagtgtgtggcagagctgtggcggaagaactacaacaacacctggacgcgactggcggagaggacctccaacctgctggagatcagggtgctgcagccagtgacaaagctgcaggtgagcaaatcgaagaggaccctcaccctggtggaaaacaagcccattcagttgaactgctcagtcaagtctcagactagccagaactcccactttgcggtgctctggtatgtccacaagccctcggatgccgatggcaagcttatcctgaagaccacccacaactccgcctttgaatacggtacttacgccgaggaggagggcctgagagccaggctccagtttgagaggcatgtgtcggggggcctgttcagcctcaccgtccagagagccgaggtcagcgacagcggcagctactactgccacgtggaggagtggctgctgagccccaactacgcctggtacaagctggcagaggaggtttctgggcgcacagaagtcactgtgaaacagccagacagccgcctgaggctcagccaagcccaggggaacctgtcggttctggagacccggcaggtacagctggagtgtgtggttctcaaccgcaccagcataacctcccagctcatggtggaatggtttgtatggaagcccaaccaccctgagcgggagactgtggcccgcttgagccgtgacgccaccttccactatggagagcaggcagccaagaacaatctgaaggggcggctgcatttggagagtccttcccccggcgtgtaccgtctcttcatccagaacgtggctgtgcaggacagcgggacctacagctgccatgtggaggagtggctgcccagccccagtggcatgtggtataagcgggcagaggacaccgctgggcagacagctctgacagtcatgcgaccagatgcttccctgcaggtggacacagtggtccccaatgccacggtctctgagaaggcagctttccagctggactgtagcatcgtgtcccgctccagccaggactcccgcttcgctgtggcctggtattccctgaggactaaagctggggggaaaaggagcagccctggcctggaagaacaggaagaggaaagggaggaggaggaggaggaggaggaggacgacgacgacgacgacccaacagagcggacggccctgctgagcgtgggcccagatgctgtctttggcccagagggcagtccttgggagggcaggcttcgcttccagaggctctccccggtgctctaccggctcacagtgctgcaggcaagcccccaagatacaggcaattactcctgccatgtggaggagtggctgcccagccctcagaaggaatggtaccggctgacggaggaggagtcagcccccatcggcatccgtgttctagatacaagtcccaccctccagtccatcatctgctccaacgacgcactcttctacttcgtcttcttctaccctttccccatctttggcattcttatcatcaccatccttctggtgcgtttcaagagccggaactccagcaagaactctgatgggaagaatggggtgcctctgctgtggatcaaagagccacacctcaactactcccctacttgcctggagccccctgttctcagtatccatccaggggccatagactaagcgggtgatgccccagcggatgttggccacggaggagctgaggctctccctttctctgtgattggacagttgacagcacccaaactctggggtgcatgtgtgtggaaagttgtcagacttgaaaagtgttccaagttcccagtcagtcacagagacagactgcctctcggtggcagtcttggttggttagctatttgcgcgcaaatgttgtgatcctgccattatagatttcttgtttctgtttttagtaatgtagtgagtagctccaggtgccacatctactcacagatttatctagtattctcagatagatgttacagggcttcttattctttgtaatgtactctttttaaatccctttagtttaccctttttggattccttaatgtggacgaatttctcttacgtacaactgacagcaaaaggaagggcgaactttctagtgacaaggaatctcttccaagactttgtttttgcacatttgaaaatgccacccatggatcaaaatatacccaaacgttttttactttcttaacaagacttaagaattgtgtgtagtgtgggcaaaatttgtatgttgtcttttccctcagctggagttattggaaccactttgtagtcaagacgaaagcactgaattttgcttcaaagaactgtgtatgtacaagagaaatcctgcataaccccattaggagtagatggtgcccggcctatctgtcagggaggcaaaaaaggcttcatcccatccttgccaaaaaataagaaaactgtcttggagaatgggtcagaagccccaaacggcacacactttccaaattaaagt

SEQ ID NO: 34 - DNA, 2398 bp, Homo sapiens
  misc_feature: Incyte ID No 747515CB1
agatggcgtccctggatcgggtgaaggtactggtgttgggagactcaggtgttgggaaatcttcgttagtccatctcctatgccaaaatcaagtgctgggaaatccatcatggactgtgggctgctcagtggatgtcagagttcatgattacaaagaaggaaccccagaagagaagacctactacatagaattatgggatgttggaggctctgtgggcagtgccagcagcgtgaaaagcacaagagcagtattctacaactccgtaaatggtattattttcgtacacgacttaacaaataagaagtcctcccaaaacttgcgtcgttggtcattggaagctctcaacagggatttggtgccaactggagtcttggtgacaaatggggattatgatcaagaacagtttgctgataaccaaataccactgttggtaatagggactaaactggaccagattcatgaaacaaagcgccatgaagttttaactaggactgctttcctggctgaggatttcaatccagaagaaattaatttggactgcacaaatccacggtacttagctgcaggttcttccaatgctgtcaagctcagtaggttttttgataaggtcatagagaagagatactttttaagagaaggtaatcagattccaggctttcctgatcggaaaagatttggggcaggaacattaaagagccttcattatgactgaattacactcatcctttggaagagtgagcaagcagtggcagtttttcacagctcatcttgctgtgttcaattattaccatcacagccttttaacaaaatcatcttaaaatgctacccttcagccttaccctttaatggaaaaatgaaaggaagtgacaatacgggaggtccaaactttgtccctgttctctgtgttccttacctttctgtccctgtgtatagattatgtaaaagccttgtgtaaatatgagatgttgtcaaaatgatgcagtaaatgagcaatgacagtgtactgcagagaaaatttactcttgcctagaactggagggtttttatgggtctgtaattttcccacactcattgctgaaagcttaattaagtacttcaaaaacgtatctccattgttttaccttcttgaggggaacggtcttgttaaccagccctgagttgtctaccccaaacaatctctgtcattttcaaagatgcaaaatggtgttatttaattgtctccaccattgtcacacacaggaatgcctaataatagcaacccttgtctccctcttctctcctttgcaaatgggtcagtgactggaagaggcggactaatagccagagttaaatataaatacaaattaataatacatagagaacagcaataccagaaaaaaagaattctggtaaaatgatgtgaaaaattgacagctccctcactcttaaggttgctgctatatacagtctaggttttctgtttggaaataggtagggtaaaatctaagacctgcacaagggcagtgagagacatttacagcctcctctctatttgtttttttaaggaaaagtcaactcctgaaatgtcccttagctataatcagaaaactaagaatattattctgtgtcaacaatgtatttatggagagaagtaaaaataagttccacagcaacacatttacatgaattatgaactaggattcttggatttcataatcactccaaagctttttggggatggtgtgtgtgtgtgtgtgtgtccgtaattgttcatcattactttttttaacctatctttttgcttgcttaatcttctctgccccagttcttcctgagtatatgaatttgatttctaaaaacaatttattcaacactctatcacccgtattctctctctggctctgctgcaggtctctgcactcctgattttgctccatacttttaaataacaataataaatatacagttttcatttctcatttaaaaaataggtggaaaacaccaaattcactttctgttccattttaattaggtttcagaagtatcttggaaatcaaggtatgctgcaagaggatattaaaataagaatgttagagagtccttttagaaaggcagtcttgggaatttggaaaaggatttttaaagcagctttcatcagctagactgttctccagcctttctttacttcctctttgaaaggacatagccctggtttctttttgacacaaaggaaagctatgcaaatcatgatctgtatgtagaattggtcacaagtatgttgttgcataggctgtacagttgggaagtagaaaccaaatgctacccagagacctaacccaattaagctctttgtatttctccttcaagaaataaatcttccttccttctttcttcctctttcttttaattttaaaaaaa

SEQ ID NO: 35 - DNA, 2533 bp, Homo sapiens
  misc_feature: Incyte ID No 7495641CB1
gcaagctgatagctgagactgtgagactgtttttgtccactcttctgaatcactgccacttgggtcagggaccacagccattgccacccttggcccatctctctgggtgcgtgccttgagcacacctataaaaagtgccatgtgcaattgtcttatcttttatgatctaggctttgcctagggatcactactccttaacgggctggctggggcgatgaggaaaaactcctttgctcctgtaaggccataagtggctgttaacagattttcaaatgcctgaagagattgctgagacctgctagagtcatatgttcggggaattaagtctttatcctagacaacaaggtacagatgcaaactgcagtgttattggagggtcaatcggcaaggatatgattatcccaaaatggagttcatcgaccctagctttcctttagattatatataaataaaagtgcagtcctcttctaatggccacagttggttttcttgtagcccagaaagtccaaattaaaggaaataaattcagttttatgttagccttccttggtgcatcagggtgtcagtggaaataggatcaggtggtgtgtgtgtgtgtgttttgtgtgtgtgtgtacacatgtgtttatatatacatgtgtgagggaaagtgtgtacatatatgtaggattgtaaccagacggaaaagaatgaggatctccagggtgtttgaatcagcaacagatttgtgttttctaacatgcatttagttggagaggcatggttctgtttgttttgttttgatctaatttgccattggaaataggtacagttacacagagaaggaagaactaggaaagtgagatccatgaaactaaatgagcagctgtcagaatccagtgtggctgagcctacctagcttatgaaatctaacccagggttccctgagtccaagaccacttagattattaagattttgaacgtccagaggagtgaaaagtctgttttctggacgtaagccggagctgaggataaagccagaggccagtggattaggtgtatggaatgtggatggagagggcttgtgtgggatgtggccagggagtgggtgaggaaggccgcttctaaatggcctgtaaaaacttgagattggatagacgaaaggaaatggagaaattaaagaattggagaaactagttatctgtgttgctgactttgggacccatccaagactcctgcccttggggtgttccatggtggtttcttcctgcctgggcgccaccctttccccagttcaggccctccctggaggactagtttgtgtattggcatcctccccagtggacccaaaccagcgcatacttggtgtgtggagatgggagacaaaggacagatctaggagccttgaaggatcaccagccaccgaccctccatcagggccaactgggcaggaaagggaacattgcagacctgatttcccgacgatgtcaccctgtcctccctccttgcttcttgctctgctaactcaactctgccttcctctttttcattcttctactctgccctatatggaggacaaatggacaccaggggtgctaaccttattggtgcctgccccagcctaccccaggtgccagcagactctcgtgcacaggaggctcccacagttatggagccaggaaagaatttctctgcactggatggactgtatattgagattaaaaattatattccttatattcctgcttatatcaatgctctctctgtaaaacctcttcctagcctcatttctctcaactgatcttgtttaggcgttgtattccttttatttactctttgcttgactgcttcctcctaaccctctacccactagcactctacttcctaaagctgttgtgtcattaactctgttggatcaactctctgggaaaagattctgttaatgtaagtgcacttactccctggatgttgtcactagtctagtggcttttgctaaataaacctttcttatttctagaagcttttccattgtcttttcttgctgcttctattccacctgctccttctctccccctcacttccaccccacttcccaagccaagtggcatcagctctgtgagatggttcccaagggtatgggattgaggaaagattcacaagttccctgtaggaggagaggaagatggtggcttcatttgcactttatatagatcagccctgcgggatcagaggtatcagtggttcttaactaggcctgatttttcctcaccaggggacatctgacaatgtctagaaatgtttttggttgtcacaaagctactggcatgtagtgagtgaagccagggatactgatcaatatcctacaatgcacaggacagccccccacaacaaagaatcatccagtcccaaatgtcagtagtcctgagattgggaaactcgtctacacccatttttgccgactctgtcaatcccaggttttttcaaactccaaatatcatttgaatatgtgggtagtagcagccaagtgggtttctcgggggagggggtcaggggc

SEQ ID NO: 36 - DNA, 1424 bp, Homo sapiens
  misc_feature: Incyte ID No 2937340CB1
ctctttttttgaaaaatttaaacatagtgagttggggggagtctcactatgttgcccaggctggtcttcaactcctggcctcaagtgatcctcctgcctcagccacccaaaacactgggattacaggcatgagccaccatgcccggcccccttcagctttcctgaatttcagattcctctttgatgaagtggcactaacaataatacctatcttgaagggctgctgtgaaggactgcaggacgtgatgcaggaacagtgcttattagtttgttacgtgttagtttattttccctggatctgagcgaagaagagccagggtggaagagatggactctgtgggtctgggcagggcctcgggggttggggtcggggccaggcagcctcacaatggtctagaactctccctcactgtaggttctcacctacttcgactcctctccctgtcccagggaggggagaaaaggtctgggattcattgccaggaggggcttcccccaggatttcctacttctttctttactgctgtgcttgaagctcacaggagacccctcaagaggtggtcaccggcccacagcccaccccatcccccacccgccacccccaccgtaccaaatgctagctctgccctttcttctgtgtttttcccatcaagagagatggttgtggtcatgaagttcttccgatgggttagacgggcttggcaaaggattatttcctgggttttcttctggaggcaaaaaattaaaccaaccatctcaggacaccctgactccaagaaacactcattgaagaagatggagaagactctccaggtggttgagactttgaggttggtcgagctcccaaaagaggctaagcccaagttgggtgagtcccccgagctggcagatccctgcgtgttggccaagactacagaggagaccgaggtggagctgggccaacagggccaatccctactgcagctgccgaggacggccgtcaagtctgtctccacgctcatggtctctgccctgcagagcggctggcagatgtgcagctggaagtcatcagtgagttctgcctcagtcagctcccaagtgaggacgcagtcacctttgaagactccggaggctgagttgctgtgggaggtgtacctggtgctgtgggccgttcggaaacacctgcgccggctgtaccgcaggcaggagaggcacagacggcaccacgtccgatgccatgctgccccccgacccaacccggctcagtccctgaaactggatgcccaaagtcccctctagggggaaccccagacccttagagagtcctgacctcactcttacctggggtcccatatcagccccttcattccatgtattccagttgtaaaacaagtatcaaaatattgggaaataaatatcagatagttctgaaaaaaaaaaaaaaa

SEQ ID NO: 37 - DNA, 2448 bp, Homo sapiens
  misc_feature: Incyte ID No 3765326CB1
gggacggcggcgggaagatggcggcctccaggaatgggtttgaagccgtggaggcagagggcagcgcagggtgccggggaagctcgggaatggaggtggtgcttcctttggatcctgccgtccccgccccgctgtgccctcacggacccactcttctgtttgtaaaggtgacccaagggaaagaagaaactcggaggttttatgcctgttcagcctgtagagatagaaaagactgtaatttttttcagtgggaagatgaaaagttgtcaggagctagacttgctgcccgagaagctcataaccgaagatgtcagcctcccctgtcccgaacgcagtgtgtggaaaggtacttgaagtttattgagttgcccttgactcagagaaagttttgtcaaacatgtcagcagttgttgttaccagatgactgggggcaacatagtgagcatcaggttctgggtaatgtgtccattacccagttaagaaggcccagtcaactcctttatccactggaaaacaagaagacaaatgcccagtatctgtttgctgatcggagctgtcagttcttggtagacttactttctgccctcggattcagaagagtactgtgtgttggaacaccaaggttgcatgagctgatcaagttgacagcatcaggtgacaagaagtctaacattaaaagccttttattggatattgattttcggtattcacagttttatatggaagatagcttttgccattataatatgtttaaccatcatttctttgatggaaagactgcccttgaagtatgcagagcatttttacaggaagataaaggcgaaggaatcattatggtgacggatcctccgtttggtggcttggttgaacctctggctattacattcaagaagttaattgctatgtggaaagaaggtcaaagccaagatgacagtcacaaagaactacccattttctggattttcccctatttttttgaatcccgaatttgtcagttttttccaagcttccagatgctggattaccaggtagattatgataatcatgcactttataaacacggaaagacaggtcgaaaacagtctcccgtgcgtattttcaccaacattccgcccaacaaaataatccttcctactgaagaagggtacagattttgctctccgtgtcaacggtatgtttctctagagaatcaacactgtgagcactgtaattcttgcacatccaaggatggcaggaaatggaaccattgctttctctgtaaaaagtgtgtaaagccttcctggatccactgtagcatctgcaatcactgtgctgttccagatcattcttgtgagggccccaaacatggctgctttatttgtggtgaactggatcataaacgcagtacttgtcctaacattgctacatctaagagagctaacaaagctgtcagaaagcagaagcaaagaaaaagtaataagatgaaaatggagaccacgaaaggacaatccatgaatcatacatctgctacaaggagaaagaaaaggagggaaagagcccatcaatatcttggctcttaaatgtccagtgactggagaataagaaagatttatggtccaacctttgatgccattttctgaaagtgccacactggacttaaattcagctgcttccagaggtgtgcacctttctgagctagataacaggcaggtggcatttgctggtttacagtcccttatctgcctctctggagacttggggagttgttccatcttcagccagtttactagttctttcagcattcatttttcctcactttaagtctctcccagtcacattgttctatttttatgttttcattttttttgagatggagtcttgctctttctccaagtctagagtgcaatggcacaacctcagctcactgcaacctctgcctcccgggttcaagcaattctcctgccttagcctcctgagtagctggaattataggcacatgccaccacgactggctaatttttgtatttttagtagagacggggtttcaccatgttggccaggctggtctcgaactcctggcctcaagtgatttgcctgccttggcctcccaaagtgctgggattacaggcatgagccacctcacccagctccggtcacattattctagcctttttatccaaaaaatgggcagaccattcttttgcagaagatgtcagaagaaaatgaaagtgaaaacctatgcagtttgtttaccgtgaacataaaagtatgttaatttaaataggaaaatattttatgttaaatatgaaacaatagtttaaaccccacttgaaaagtatagcgtacaattatttttagactgaatttgaagatgatggaaagggtttttacaaaggcactgtttagatatacagattataattataattacattattattaaatatttattcttcaagtaaaaaaaaaa

SEQ ID NO: 38 - DNA, 1616 bp, Homo sapiens
  misc_feature: Incyte ID No 948883CB1
aaccgtcaggttgaaccacgccacgctgccgatttttacactgtcgggaatatctttaatcagaaactgcggcgctaccctggacagcaacagatgggtagcgagtttgaccgtgctggaattggcgcggccatttttctcaagatggcggctcaaacacgccgtaacctctgagggtcgctgcccccagtgatcaggggcatgcaaaggccattcggcgattttgttgcgctcaggtgccctgagtgactccgggtcgagccccaggtgaatcgccgctaatacgtagtcgtagaggctgttgtccgtggccgcgccctgcaggtgggcctgcaaagccaggccgagtgctcgggccttaggcgagtcgagcagcttttgcatcgtcacggtcgggtcttgcaagtcgctttggctgaccgaactgccgctcagcagataggtcagtacgccattgccatggggtggcaacggcaaacctggcaaacctgttgtgtcggatttgaggaaggcgcgaatgctgtgctggtccgccttcgccatgggaattggccaggtgagagcgccaccgaaatcacccagggggtgctgtaaggccttttgctccagtttctgcgcgagtgccgccatctcatcgaccgtgcgcggcagttcaaaacccaagtgcgtgatcactgtcgccagggtttgcgctgccggctgcacgtgggcgtacgccgagtctttatggggcgtcagacgactattgctcaacccgaatgagacattgtccggggtaacgggcttgccatacgtccgggtatcaatgccgcgcagttgctcggccaggttgcgcaggttctgctggtctgccagttgtgtgcgcaaggtggcgggggcaggtgtttgacccgcgcgtggggaatctgaggcggtcgtagtgaacgtgtgtgaagcgtcttgcgcaaccggcgcgggagcttcgacgacaaaggcgctggggttgtctgccagtgtgcgggtaggggcggttatgctcattgggggtaccttggggggcaaaaaagacgccttccaaaagatcttccgtgttcgtgaggcgctgtcctgagtgccgcaacccttcacctacgttccgccgcatctgtatgcacgtatgtaaggggtctgatgaaaccggcccgaattggcgtgtaactcactcgcagcctgtaggccagcgcttcaaaacaacgcacaaacatagccttgccccctgatattcgggcaaactccccctttttccgcacttggccaaggcatgcccatgactcccgcgtcgtccgtcgatgaaaagagcttccgtaccctgttgagccgaaacgtcgcgttacccttgggcgtcggcgtgctcagcgcggtgtttttcgtctgcctgatcacttatttgctgtcggttatccagtgggtggagcacaccgaccgggttatcaataacctcaatgagtcatccaagctgacggtggacctggaaaccggcctgcgtggctttctgatcactggcgacgagcatttcctcgacccttacgaagtggccaagccccgcatcattggtgacctgcgcaacctgcaggagctggtggcggacaacccacagc

SEQ ID NO: 39 - DNA, 424 bp, Homo sapiens
  misc_feature: Incyte ID No 5665403CB1
taaatgattctgtataccattttcttgaaggtacaagaagattctgccgactatggggatctttgggccagtttgaggattgctttccctctgaggttctttctctctgtcagccacactttctcacccaacttcagacacaccctgccagcctttcccctactcattcactcttccccttccctcaacttaatcgtctatcccgttgcctgctgtttgtgcactgaaggcaggtggatggagtcagtcctcagttgcccctgctggccttcctggtgcttaccatcagcccaatctttgcacagtccttgttgttcttacttctctgcaatgcattccttcagaagatcagtcatcaactttttcttaattcctctgtgacacacaatgggaattcaaaggaagagatcttaaaagtcacaac

SEQ ID NO: 40 - DNA, 1782 bp, Homo sapiens
  misc_feature: Incyte ID No 7493065CB1
gccgcacgcacgcgcactgcgcccagcatgagggtcgcggctctgatcagtggtgggaaggacagctgctataatatgatgcagtgcattgctgctgggcatcagatcgttgctttagcaaatctaagaccagctgaaaaccaagtggggtctgatgaactggatagctacatgtatcagacagtggggcaccatgccattgacttgtatgcagaagcaatggctcttcccctctatcgccgaaccataagaggaaggagcttggatacaagacaagtgtacaccaaatgtgaaggtgatgaggttgaagatctctatgagcttttgaaacttgttaaggaaaaagaagaagtagaggggatatcagtaggtgctatactttctgactatcagcgtattcgagtggaaaatgtgtgtaaaaggcttaatctccagcctttagcttatctttggcagagaaaccaggaagatttgctcagagagatgatatcatctaacattcaagcaatgatcatcaaagtagcagctttgggtttagatcctgataagcatcttgggaaaaccctggatcaaatggagccttatctcatagagctttctaagaagtatggagtacatgtttgtggagaaggtggagagtatgaaactttcactttggattgccctctatttaagaagaaaataattgtggattcatcagaagtagtcatacattcagctgatgcatttgcacctgtggcttatctacgctttttagaattgcacttggaggacaaggtgtcctcagtgcctgacaactacagaacatctaattatatatataatttttgaaaagtgttttggaacattgttcattaaaccaccatttctatacaaaaaaattgcatagtattttctcagttactatgactagtttatttttttctcatgactcttatttttttagagaaacatactttcactagaagaggttagtggaaccatttattaattgggaaaatgtcgacggcatgttcattaatagtgccaactttcttggaattcactctttctctttcgttaacacatcttccctatgaccttttttttcttttatttcatctataaaccccatttctgtagcatttctttttttatcacactagtttcttttcctccttctctctttcttgctccaatccctaccaataatgtcatgatgcagaggcatttgaaaatgaagatgaaaagatggtcactttatttagccagtcaagcttattctactgggtgtcgccaaagcgatttatcatttttattttaaatatattcatggttgaagtgtttcaacgttattggacaattaggaagaatgtccttaactcttacaagttattttataactgatttttaaaatgctgtttttcagtattaactatgttgaccttagaaaacttttacaaaaaatagactttatgttttagaacagttttaggttcacagcaaaaatggagcagaaaatgcagagatttcccatataccctctacctccacacattcacagcctcccataccgtcaatgtcttataatagagtggtacatttgttacaactgatgaacctacattgacacattattatcacccaaagtccatagtttacgtgagggttcactcttggtgttgtacataatatgggttttgacaaatgtgtaatgacatttgtcaaccgttatagtattatacagaataatttcactgcttaaaaaatctctgtgttttacttattcatccctccctctct

SEQ ID NO: 41 - DNA, 846 bp, Homo sapiens
  misc_feature: Incyte ID No 7493531CB1
ccaccatgtaccccttccttctgccacggccacatgagcctgcaagagctgccaggctgacatggggctttctggccacagtgactgatccagggatgggaagagatgtagccttggcgttggaagttttcaagtcttgggtatgtctgcatgcacagtattggcagcgtgaaaatggacttcaaaggaggaatgtgtcactatgagctctctgccatgtgagggcgccatcccacaagggaaggagaggagagctggctggacagcagcactcatgtacagaatgccagtggtcccgtggtacaggctgtcatggaaatgcagagcagagccacatatggccatgtcactcaggctcacggcagaagagacttcccttgtcttagatgagacttcagacttggacttttgggttaatgctagaataagttaaactttgggagactgttgggaaggcatgattgagtttttaaatgtgagaaggccatgagatttgggaggggctgggggtgtaatgatatggtttggctctttttccccacccaaatctcttctctaattgtaatccacatgtatcaagggagggacctggtgggaggtgattgaatcatgaggcagtttcccctatactgttctcatgatagtgaatgagttcttatgggatctgattgtttaaaaatatagcactttcacctcccacctctctcctgttgccatgtaaggcgtgccttgctttcccttcaccttccaccatgattgtaagtttcttgatgctccctaaccatgagaactatgaagcaattatgtctctgtggttagcnttggngtgnaattgccttaagtttg

SEQ ID NO: 42 - DNA, 3601 bp, Homo sapiens
  misc_feature: Incyte ID No 3321454CB1
gaagatggcggccgaatggggcggaggagtgggttactcgggctcaggcccgggccggagccggtggcgctggagcgggtctgtgtgggtccgaagcgttttactcctgttgggcgggctccgggccagcgccacatctactcccgtctccttgggcagttcccctccctgccggcaccacgtcccctctgacactgaggtcataaataaagttcatcttaaggcaaatcatgtggtcaagagagatgttgatgagcatttaagaatcaagactgtctatgataaaagtgttgaagagttgctccctgagaaaaagaatcttgtaaagaacaagcttttcccacaagcgatttcttatttagagaagacttttcaggtccgtcgacctgcgggcactatcttacttagcagacaatgtgcaacaaaccaatacctccggaaggaaaacgatcctcacaggtactgcaccggggagtgtgccgcacacacaaagtgcggccccgttattgttcctgaggaacatctccagcaatgccgggtctaccgtgggggtaagtggcctcatggagcagtgggtgtgccagaccaagaaggcatctcagatgcagactttgttctttacgttggtgctctggccaccgagagatgcagccatgaaaacatcatctcttatgcagcctattgtcagcaggaagcaaacatggacaggccaatagcaggatatgctaacctgtgtccaaatatgatctctacccagcctcaggagtttgttgggatgctgtccacagtgaaacatgagggtttctctgctgggctgtttgcattctaccatgataaagatggaaatcctctcacttcaagatttgcagatggcctcccaccttttaattatagtctgggattatatcaatggagtgataaagtagttcgaaaagtggagagattatgggatgttcgagataataagatagttcgtcacactgtgtatctcctggtaacgcctcgtgttgttgaggaagcacgaaaacattttgattgtccagttctagagggaatggaacttgaaaatcaaggtggtgtgggcactgagctcaaccattgggaaaaaaggttattagagaatgaagcgatgactggttctcacactcagaatcgagtactctctcgaatcactctggcattaatggaggacactgggagacagatgctgagcccttactgtgacacgctcagaagtaacccactgcagctaacttgcagacaggaccagagagcagttgccgtgtgtaatttgcagaagttccctaagcctttaccacaggaataccagtactttgatgaactcagtggaatacctgcagaagatttgccttattatggtggctccgtggaaattgctgactactgccctttcagtcaggaattcagttggcatttaagtggtgaatatcagcgcagctcagattgtagaatattggaaaatcaaccagaaatttttaagaactatggcgctgaaaagtatggacctcattccgtttgtctaattcagaaatcagcattcgttatggagaagtgtgagaggaagctgagttacccagactggggaagcggatgctatcaggtttcttgttctcctcaaggtctgaaagtttgggtccaagatacttcatatttgtgtagtcgggctgggcaggtcctccctgtcagtatccagatgaatggctggattcacgatggaaacctgctctgcccatcatgttgggacttctgtgagctctgtcctccagaaacagatcctccagccactaacctgacccgagctctgccacttgatctttgttcctgttcctcgagcctggtggtcaccctctggcttctgctaggcaatctgtttcctctgctggctggatttcttctgtgtatatggcactaggaatggaaaagtggatcttcaagatattcttttatgttatgttcttgtgaacaaagcacaaagtttgagtgagtgccaacctatgcagatggtagaagtggcattcctggctttggcttggaagaattgacgaccatcagaccttgaagcagaacttcacagcagcctgtcctcatcagcaacccaaccaccttcatcagcaacccaaccaccttcatcagcaacccaaccacctcgtcagcaacccaaccacctcgtcagcaacccagccaccttcatcagcaacccaaccacctcatcagcaacccagccaccttcatcagcaacccaaccacctcatcagcaaaccaaccaccttcatcacaacccaaccactttcatcagcaactcaacaccttcatctgcaaacccaaccaccttcatcagcaaaccaaccaccttcttcagcaacccaaccacctcatcttggagaaggagaaggaactgcaagccaccaagtcttcatttttcagggtttgtaatcttcccaaagttttcctttgaaaataggataatgggtggaattttcagagtgattacatacctcaacatttttattaacatacaacaatgggaaagttcatcatccatatactgcagtcacttaaacacagccaattattgcaagattagaattggagatcttgtcctcaaaagtataaattgtcctttgagttatagaaaataatggaattgggatttctacatatcattattatacctattttaaatttaatggcagccaggcatggttccagctacttgggaggctgaggcaggaggatcgcttgagcccaggagttcaaggctgcagtgagctatgattgcaccactgtattccagcctgcacgatagagttagaccctgtatcttaaaaaaaaaaaaattaatggctggtatatagtaaacttaattcactgttcccattgttctaaagaatttttttaaataatgtttcattaaatcttatgatttaaccgtgcttgccctttttgccagctatgtggcagtttacggcagaactgctgtcagtgtgctggtagccctctattcatccctcctcagagcccagcttccagaactgctatcactgtgctggtagccctctgtattcatccctcttcagagctcagcttctggttgtattctccatgagtttaataatgacatgaaaaaatgtggaagccgagagagtaaaatactctgccctgtaaaaacatggaagacatgcaaacagaaaaaaaataattgtattgttttagataatacttaagacaactgtgatacaacaaaaacacaactattcctttgtgaccgatgaagataaaaagaaattctggtaaagacgggtatgcagttttttaaaatgggttaagaaattgttgcagaagaatttctaacatctgaaaataggttattatgtttaagaaggatggtctgaattgtgtactaatagcaaggtataagtttggtgtagagcctatccagtagcgtccactgtaccacttttaagtaagactcagtccacagaagctggaagattgccttcgctttaaatatcctttaccttctgcatttgacactcctcctgttacacatagattccggtccccagaagcaactctacat

SEQ ID NO: 43 - DNA, 2333 bp, Homo sapiens
  misc_feature: Incyte ID No 189299CB1
tgagtctgtcctctggggctcgccatgcccccagcctccgcatggggaacaccatcagggccctcgtggccttcatccctgctgaccgttgccagaactatgtggtcagggacctccgtgagatgccgctggacaagatggtggatctgagtgggagccagttacgccgcttccccctgcacgtgtgctccttcagggagctggtcaagctctacctgagcgacaaccacctcaatagcctgcctccggagctggggcagctacagaacctgcagattctggccttggatttcaacaacttcaaggctctgccccaggtggtgtgcaccttgaaacagctctgcatcctctacctgggcaacaacaaactctgcgacctccccagtgagctgagcctgctccagaacctcaggaccctgtggatcgaggccaactgcctcacccagctgccggatgtggtctgtgagctgagtctccttaagactctgcatgccggctccaacgccctgcgtttgctgccaggccagctccggcgcctccaggagctgaggaccatctggctctcgggcaaccggctaactgactttcccactgtgctgcttcacatgcccttcctggaggtgattgatgtggactggaacagcatccgttacttccccagcctggcgcacctgtcaagtctgaagctggtcatctatgaccacaatccttgcaggaacgcacccaaggtggccaaaggtgtgcgccgtgtggggagatgggcagaggagacgccagagcccgaccctagaaaagccaggcgctatgcgttggtcagagaggaaagccaggagctacaggcaccagtccctctacttcctcctaccaactcctgaggagcttcagttgcaagtcaatgccaaggacccaactgcagcatgttctggaagcctctccattggagtggaaaggatggctctgggtcatttgggagtggctctgctagtagagactgatggagagagccaggtggaatgccataaatcacactgagaaaatatttctggcaaacagctcctcttccagaggggagttgtgtgccaatgatggcatgacaatccagagatcataacttctttgcaagaaaacagcttctccacacatgtattttgaaacactgaagagcaaaaggggctgggacactctgaactcctgcactctccagaagtgactggtcatgaggctcatgagctcctcaaataaggtatttgccatagaactaaatattctggtggtctgtctctttgcaggacatattttctttactgtaaatgaccataaacagtatcaatgtatcactgaggccaccgaaaaggacatttctacctaggcaatcagtcagattcacagaaaaaagttgtttgttgttgtaaaggctcaagatgaaactctttccccagcagtttagtgcctgctgaaaagatccctgatggacaatacttcttggtggactccagctgccccttttattattattagagacaaggtctcactctgttgctaggctggagtgcagtggcacaatcatggctcactgcagccccgaactactgggctcaagccttcctcccgcctcagcctgcccagtaactggtactacagatgtgcacacctggctaagtctttaattttttcgtagagatgaggtcttgctatgttgcccaagctagtttcaaactcctgggctcaagcgatgctcctgcttcagcctcccaaagtgctggggttacaggcatgagccaccacacccagccttcagctgtcaccttaaacttgacagtggctcatgctgatttagttcattttccctaaaaggtttgtcccaagatctgctcccaacagttgactgtcactgacaatgttggaagtcatctggaaaagagaacctctgtggtaatgtggtctcattaaagtcaagccttgttgtgattcctgtctacctccctgaagcaaagcccttctgtttattcacactaatgagccagagctgagctaaattgaatccctgtccttggaggaaaaccacatttccagaagcatgttagtttaaaggtagtaggtgagaaatgtgttctcttgaaacaagcactttgaaatttgaataggaagttgtagtgtatataggaagtctccgcctctttcgcctagtatctctgcctttgtttcaatttgttttgatttttacagactgttttgacaatgtataaaccaaggtattttgttttttggaagtatgtaaattgtgaccttcccacaaatatataaactttaaagaaaaaaaaaaacaaaggggg

SEQ ID NO: 44 - DNA, 2016 bp, Homo sapiens
  misc_feature: Incyte ID No 7488057CB1
ggacagtacagctgacagccgtgctcaggaagattctggatcctaggctcatctccacagaggagaacacgcaggagcagagaccatggggcccctctcagcccctccctgcacacagcatataacctggaaagggctcctgctcacagcatcacttttaaacttctggaacccgcccaccacagcccaagtcacgattgaagcccagccaccaaaagtttctgaggggaaggatgttcttctacttgtccacaatttgccccagaatcttactggctacatctggtacaaaggacaaatcagggacctctaccattatgttacatcatatgtagtagacggtcaaataattaaatatgggcctgcatacagtggacgagaaacagtatattccaatgcatccctgctgatccagaatgtcacccaggaagacacaggatcctacactttacacatcataaagcgaggtgatgggactggaggagtaactggacgtttcaccttcaccttatacctggagactcccaaaccctccatctccagcagcaatttcaaccccagggaggccacggaggctgtgatcttaacctgtgatcctgagactccagatgcaagctacctgtggtggatgaatggtcagagcctccctatgactcacagcttgcagctgtctgaaaccaacaggaccctctacctatttggtgtcacaaactatactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccgttcaggagaaaacctctacttgtcctgctttgcggactctaacccaccggcacagtattcttggacaattaatgggaagtttcagctatcaggacaaaagctctctatcccccatattactacaaagcatagcgggctctatgcttgctctgttcgtaactcagccactggcaaggaaagctccaaatccatgacagtcaaagtctctgactggacattaccctgaattctactagtacctccaattccattttctcccatggaatcactaagagcaagacccactctgttccagaagccctataagctggaggtggacaactcaatgtaaatttcatgggaaaacccttgtacgtgaagcatgagccactcagaactcaccaaaatattcgacaccataacaacagatgctcaaactgtaaaccaggacaacaagtggatgacttcacactgtggacagtttttcccaagatgtcagaacaagactccccatcatgatgaggctctcccccctcttaactgtccttgctcatgcctgcctctttcacttggcaggataatgcagtcattagaatttcacatgtagtagcttctgagagtaacaacagagtgtcagatatgtcatctcaacctcaaacttttacataacatctcagggggaaatgtggctctctccaccttgcatacagggctcccaatagaaatgaacacagagatattgcctgtgtgtttgcagagaagatggtttgtatgaagacgtaggaaagctgaaattataatagagtcccctttaaatccacattgtgtggatggctcttgccgtttcctaagagatacattgtaaaacgtgacagtaagacattctagcagaataaaacatgtactacatttgctaaa

SEQ ID NO: 45 - DNA, 1280 bp, Homo sapiens
  misc_feature: Incyte ID No 7486411CB1
ttcttctagtacctgggaaacagttaggcagagttcaggtatggatctaagatggcactgtcagtaagagcagaatcaggactggagtccaggcagcctggctcctgaatcctagctcctaactatgagaccctcatgtctttaggaatatggagttctgactgtgatagggtgaaagagggaagtcctcacagtttgcctgttgatctctaattttctacatcatggtaatctgtcacaaaaggggaggccacacctggcccagcccctttcacccactctcaatcccctagcaggtgccacacagaaggcgtgtcctcgtccattggttcttgcatgacaatcagagggacacggatcctccctttcatcaatgacccagagctgcgggtggaattctacactgggatgaatgagaactcagacatcgccttccatttctgagtgcactttggccattgtttggtcattgacagccatgtgtgtggggcctggaagtgtgaggggagatgccacaatgtgttcttcaaggacggcaaacaatttgatctgagcatcttggtgctagacaatgaataccaggaaagcttgctgggaggagcatggaatctggaatgatgccagagggcaaagctgaaaggggtcatttaagtgctgcaactcagagattcactcagaagactggacacaattccgaaggtcgcccagaaggagaggacaatgtcatttctaactgtgccatacaaactgcctgtgtctttgtctgttggttcctgcgtgataatcaaagggacactgatcgactcttctagcaacgaaccacagctgcaggtggatttctacactgagatgaatgaggactcagaaattgccttccatttgcgagtgcacttaggccgtcgtgtggtcatgaacagtcgtgagtttgggatatggatgttggaggagaatttacactatgtgccctttgaggatggcaaaccatttgacttgcgcatctacgtgtgtcacaatgagtatgaggtaaaggtaaatggtgaatacatttatgcctttgtccatcgaatcccgccatcatatgtgaagatgattcaagtgtggagagatgtctccctggactcagtgcttgtcaacaatggacggagatgatcacactcctcattgttgaggaaaccctctttctacctgaccatgggattcctagagcctgctaacagaataatccctcctcaaccccttcccctacacttggtcattaaaacagcaccaaaccataaaaa

SEQ ID NO: 46 - DNA, 709 bp, Homo sapiens
  misc_feature: Incyte ID No 2005762CB1
gaaagttttcagtgatggtgcagtgttttttttgtgtgtgtatgtgtgtgtgtgttttcaaactcatgggcaaattcaagttcagatggtgcacttttcattgattaatgttatagcaaatgtcagtttgttttcttcagtttctgctgatggttttaactggcacagaaagtatttattcaaccttacaaaactgtgtaagttgtattgtgatacagtttattgacttgtacagtattgtcattacaacccattcaggtatgcatgaatctgaagcagaacaccatttaagactggtgctgtataatataattcctacagatgtgggtccagggaatagaactgagcctgtcttttttttaatgttaagcaggttacccccagtggggcttctactagacatctctccatttggtctcttcttacactccaacccagcaggaacagttaataattggatgtttataaaatgggggtaaaagggtttactgtgatggatcctggctatccctctaggaggagacctttgcttcagcaatggtgtcttcatcctcgcagcctgaagctgcttcatttccttaggtctgttgtgttttctgtaaagtgttaggaattctggatatttttgtaaaagaatcaagatttgtataaaatgttgtttacagatcttttaatgaataaatacataaaccccccacagaaaaaaaaa

SEQ ID NO: 47 - DNA, 1699 bp, Homo sapiens
  misc_feature: Incyte ID No 2514091CB1
ggactgacccaagatgccgttgactccagagccgccctctgggcgcgtggaggggccccccgcatgggaagcagccccatggccctcactgccctgtgggccctgcatccccatcatgctggtcctggccaccctggctgcgctcttcatcctcaccaccgctgtgttggctgaacgcctgttccgccgtgctctccgcccagaccccagccaccgtgcacccaccctggtgtggcgcccaggaggagagctgtggattgagcccatgggcaccgcccgagagcgctctgaggactggtatggctctgcggtccccctgctgacagatcgggcccctgagcctcccacccaggtgggcactttggaggcccgagcaacagccccacctgccccctcagccccaaattctgctcccagcaacttgggcccccagaccgtactggaggtcccagcccggagcaccttctgggggccccagccctgggaggggaggccccccgccacaggcctggtgagctgggctgaacccgagcagaggccagaggccagcgtccagtttgggagcccccaggccaggaggcagtggccaggagcccggatcctgagtggggcctccagccacgggtcaccttggagcagatctcagctttctggaagcgtgaaggccggaccagtgtggggttctgaatccccagggttccccagagacccccgaggcaggccttgcctcagtgggaccggggaccccaggatccagcattaggattgagactgccccagcgaagatgcccttcccaggctccttccacctggagtccccctccccgggtctgggtggtggccaggctatgtggactaggggaagcccagcagtgcctctgctcagctacctgggctgtggctcagagacctgggggtggagccaatgccaggccagaagccttcaagatcgcatccagatgaagaacccaaggtactagatagtcaggaaatggcatcgaccagccacctccaccttctttcagtgtttaccgaagccaccaataccaaagagaacgggtcctgcggtgctgaacagcctcggtgtggcgatgacagctggcaggagatgacaggaatccagtttcccagagccacaaatcctgttctccttggccactcacccactgtgaggtcctctaggaaaatacacaaagagaggaccagaccaggcagaggaacattttgtttcatatgaactgtggctttgacccccaaactgcaaggaggaacttgctgggccaagctgcagcggcactgtcttgctggagtggggacctagagtcagagaaaacccacaggctcctctgcccattctcctccatctgcacacgtctcagcctcggaccctcaccactccatggtgaggaaggccatggccaggggaaactgagtttcatccaatgtggagaggagcgttgtcctagagcagggcaactcccaaactgtgacctctgatcatcgtcccttccagcttgctggagtgtccagagagacagatttgccacaagctaggcttacttataatgctccaccctacagaaatgggaccccaagtacccgatcttccctttaggagaggcaggcaggtgggtgagcagcagatgtagtttccatttccctgggggtttaattttccaaacttgtctttttttttttt

SEQ ID NO: 48 - DNA, 3060 bp, Homo sapiens
  misc_feature: Incyte ID No 2726954CB1
gcaggaggcggaagaggtgctgtgcaggaggcgggcgggcgcggttctttccggaaggattgaatctcctttagccccgcccgcctccgtagctgcctgaagtagtgcagggtcagcccgcaagttgcaggtcatggcgctggctgctcgactgtggcgccttctgcctttccgacgtggagccgccccggggtctcgtctccctgcggggacttcgggcagccgcgggcattgcggcccctgtcgattccgcggcttcgaggtaatgggaaacccaggaactttcaacagaggccttttactctcagctttgtcgtatttgggttttgaaacttaccaggttatctctcaggctgctgtggttcatgccacagccaaagttgaagaaatacttgaacaagcagactacctgtatgaaagcggagaaacagaaaaactttatcagttgctaacccaatacaaggaaagtgaagatgcagagttactgtggcgtttggcacgggcatcacgtgatgtagctcagcttagcagaacctcagaagaggagaaaaagctattggtgtatgaagccctagagtatgcaaaaagagcactagaaaaaaatgaatcaagttttgcatctcataagtggtatgcaatctgccttagtgatgttggagattatgaaggcatcaaggctaaaattgcaaatgcatatatcatcaaggagcattttgagaaagcaattgaactgaaccctaaagatgctacttcaattcaccttatgggtatttggtgctatacatttgccgaaatgccttggtatcaaagaagaattgctaaaatgctgtttgcaactcctcctagttccacctatgagaaggccttaggctactttcacagggcagaacaagtggatccaaacttctacagcaaaaacttacttcttttaggaaagacatacttgaaactacacaacaaaaagcttgctgctttctggctaatgaaagccaaggactatccagcacacacagaggaggataaacagatacagacagaagctgctcagttgcttacaagtttcagtgagaagaattgagaacttttcagagaagatttatgaaatagctaataaacattgccttttcttttaattctaaacttaatatatgaactataactgttctacggctttttaaatgttgtgaccatttaaccgtgtaaatataaaatattctaggcttcttcacaaataatagggtaaaataaataatcgccataagagtggtagaaataaatctccatggctcaggcaaagagattattttgcatcctggataccagcaatgcaaaatggtatgagatttctaaggattgatcacattgggatgggagatcaagcaaagaaatatttgtagaggaggggaaatggatctataggggatatacagggggatggattttcaaattggattgattctaagttgaaatcttgaagagaaggtgtggtgacagtggttaggatgttgtgggttcctgacataaagtagttaaatgatatatcttggagctaacctgtgtaagtaaagaactaagtaaggagatgactaaaaatggagtagtttccttttttatttttttgagacagagtctcactttgtttcccaggctggtgtgcagtggcacaatctcggcccactgcagcctccgcctcccgggttcaagtgattctcctgccttagcctcctgagtggctgggattacagggttgtaccaccacactcggctaacttttgtatttttggtagagatggggttttgccatgttggctaggctggtctcaaactcctggcctcaagtgatctgcccgccttggcctcccaaattgctgggattacaggcgtgagccaccgcacctggccagtttactttaaatgtggtgtagtctcatggtaaactgaatttgtcatcagatgcaaagttctattccctaatggaatggaaggaacacaaaacttaagagtgaaatggaatactaagatgtttttaaataggcaggactatgctactcacttgaggctggagtgccaccactgcaaaatctttttaagttttgtaaaaaggagcatcttgaatccacttagataaagagagactgtgtgtgtaggtggatttttcccaaaggatttgggaattgtaatgttacaatgaactgtatggatatgtttgtcatgtacattttcaaacaaaaaggaaaactgaaagtagtgatctttgtatacccatctcttagattcagtgattttgctatataggttgtgtatcccttatctgaaatacttgggactagtagaagcatcttggatttgggatgtttttccaaattttggaatacctgcatacacacaataagatatcttggagatgggacccaagtttaaacacaaattcacttgtttcatatataccttatgcacatagcttgaaggtaactttatataacattatttttaataattttgtgcattgagaccaagtttgcataccttgaaccatcagaaagcaaaggtgtcattatctcagccactcatgtgggtaatttgtggttggttgatgtcaccatcattcctgactgaatgtatatgctaccaataagcagttattttcttatacttattcatgcataagtacttaacagtaaaaaatatgacataactcgcacaggaacaaggatggcaaaaaaaaaaatatgacacaccactgatacagtgaaaaaataatgtggtcagggtagctaggcaacagtagcatcaccagaaacctgtatcagctgttaaacggcaacaacaatggcaggctttcagtttcccacttaatgatgctgtattttaaaaggttattgtatactgtaattttatttttgtaggtgaagagaaacagaagcagctgaagggccaggaagtgggtctttctagggatgtggcattctgctggatggctttttaaaatgggttttttcctttagggagaccgaataaactgtgttgtgcacctgcaaaaaaaaaaaaaaaaaaaaaaaa

SEQ ID NO: 49 - DNA, 746 bp, Homo sapiens
  misc_feature: Incyte ID No 5406015CB1
tgtgcacagcccttctaaggaggcctgctggcgcacagtaggcatggtgtacatgtgagttgggttactgggcacaattcaccatgaaaggcatggcttgctcagtctccatacctgcttttatggagctcttggtctcattcctggagcctacccattcatcacaggcccaagccggacccccttccctggtcccagtctctggacagcttgctcctgtaagaaggcccttgtacctctgccccttttctcttatgggtccggggcagcattttcaggtggggatgttagatagagtgacttcaactccagagcagtggccacggttaccgtggtatttcccgaaggtgcagcgttgccatggcgtctccatgacgaccagagcatctcattcattatcctcagcaccttggcagggaaagctgggactagggctaggggagtgcagtgggagcctggctgagggctgtgggccactggcctttgtggctgaggttcccctcatgcccctgcctaccccaagccaaggttcctctgcaggctctggagttatctcccacccatgggttctgggttcacatttcagctctgccactgaacagttgtgtgatgtcgggcaagtgacacctctctgagccttatttactcaactgtaatgtggttacacttcagtgtagggttctgcatggggttacggctggggggggggccgccgtacctacttgggccgggggccgggcggttcc

SEQ ID NO: 50 - DNA, 2303 bp, Homo sapiens
  misc_feature: Incyte ID No 2850658CB1
cgctgcaggtgtgcggcccagtccgagacagcagatgaggagactgtccttcctgtttcgcagatgaggaaactgaggcttagagaagtttggcaaattggctaagttcctacagctaattgtgggattagtgatatgcttttctaaagagtagaaaagtcgaagatgtctagacagaacttagtggctttgacagtgactacccttctgggtgtggctgtaggggggtttgtcctctggaaaggcatccagcgccgccgaaggagtaaaacgagtcctgtgacccaacagccacagcagaaagtgctgggcagtagagagctgccccctccagaagatgatcagctgcactccagtgcccccagatcctcgtggaaggaacggatccttaaagcaaaggtggtgacggtgtctcaggaggcagagtgggatcaaatcgagcccttgcttagaagtgaattagaagattttccagtacttggaattgactgtgagtgggtaaatttggaaggcaaagccagccctctgtcacttctacaaatggcctccccaagtggcctgtgtgtcttggttcgcctgcccaagctaatctgtggaggaaaaacactaccaagaacgttattggatattttggcagatggcaccattttgaaagttggagtgggatgctcagaagatgccagcaagcttctgcaggattatggcctcgttgttagggggtgcctggacctccgatacctagccatgcggcagagaaacaatttgctctgtaatgggcttagcctgaagtccctcgctgagactgttttgaactttccccttgacaagtcccttctacttcgttgcagcaactgggatgctgagactctcacagaggaccaggtaatttatgctgccagggatgcccagatttcagtggctctctttcttcatcttcttggataccctttctctaggaattcacctggagaaaaaaacgatgaccacagtagctggagaaaagtcttggaaaaatgccagggtgtggttgacatcccatttcgaagcaaaggaatgagcagattgggagaagaggttaatggggaagcaacagaatctcagcagaagccaagaaataagaagtctaagatggatgggatggtgccaggcaaccaccaagggagagaccccagaaaacataaaagaaagcctctgggggtgggctattctgccagaaaatcacctctttatgataactgctttctccatgctcctgatggacagcccctctgcacttgtgatagaagaaaagctcagtggtacctggacaaaggcattggtgagctggtgagtgaagagccctttgtggtgaagctacggtttgaacctgcaggaaggcccgaatctcctggagactattacttgatggttaaagagaacctgtgtgtagtgtgtggcaagagagactcctacattcggaagaacgtgattccacatgagtaccggaagcacttccccatcgagatgaaggaccacaactcccacgatgtgctgctgctctgcacctcctgccatgccatttccaactactatgacaaccatctgaagcagcagctggccaaggagttccaggcccccatcggctctgaggagggcttgcgcctgctggaagatcctgagcgccggcaggtgcgttctggggccagggccctgctcaacgcggagagcctgcctactcatcgaaaggaggagctgctgcaagcactcagagagttttataacacagacgtggtcacagaggagatgcttcaagaggctgccagcctggagaccagaatctccaatgaaaactatgttcctcacgggctgaaggtggtgcagtgtcacagccagggtggcctgcgctccctcatgcagctggagagccgctggcgtcagcacttcctggactccatgcagcccaagcacctgccccagcagtggtcagtggaccacaaccatcagaagctgctccggaaattcggggaagatcttcccatccagctgtcttgatagctgctttcctcccagttaggacaagtgggaagctggagccaaggttgaagagtcacctcttcccattttagtacaccattaattgtcaaagcctgtgtgacacaactcagaatactaacctagactaatcccaggatgcttctgctggagcaaagatattgtttgaaggagagtttatggttttggattttaaacgggcagggtcttttttcctctcatttttgtggacaagagaggccttcgcctttatttttactctccctcttctg

SEQ ID NO: 51 - DNA, 604 bp, Homo sapiens
  misc_feature: Incyte ID No 6579653CB1
ccaaggacggccccaactgcagtcccctgaccctgacatgccccaggcgggcctcagtgacagagattttccaggccaggctccccctgccctggggggattggaggaatcctagcggagcacaagctgcccagcacagcccctccagccctgaagcctgaggagtggaacgtggtggattttccctacatgcggctttggctaaaccgcacgttcccctccctcctgagggtctgtctcctcatccacccgctagtgcatctccagcttctgtacctcagactcaccttgaaagtccacttgacatccatcactgtgcagtcagtcaggaaaaggaagccacactcgttatttcaaacagcaacggaagttaatacagagaactggttacacaggtgctggaggctggaagagaagacgggcacctgaggcacctgacgttgtgaacagcaggaagcagcttccactcccagggctgggggaacaaatgaacagaggttgggctgccagaacccaggagcacagagaatggatggagtggcagagccagtattgggagtgcccaaagaagtggtgcgctggagtagccacccccctctatggt

SEQ ID NO: 52 - DNA, 1061 bp, Homo sapiens
  misc_feature: Incyte ID No 6819648CB1
gggaattaaactggtgccggccgttaggtccacccagcagatctacccggcaggcacagcaccgcgggtccggagagcgccagtgcctgtcaccaggagcagagtgaacccctcgtgggtgccagctcctcctgcccctcctgagcctgacggggctacggttttcagtggctgcgacgccacaggacctgtgaggagagactgcaccctgaaggtctggcggcgagcggatcctaaacaaatgtgaggcctaggagcgccgtcctgaaggcactgctctccccagggccagctcctggctatggggtcagacaggtcgctgggtcctcacagccagcgcaaacacagatccaggcgtggtcgtcccgtgtccagggagctgctaccttgtgctgctgggacctggcatcgagtaggtacagccaagcccacaggaacggagccgtggcttccagggtcgtgcaaccctggcctgccctgggctggctgagtgggcccaccaccctgggctggcacttgggctcccctcaagcagactgcctgtctgcagcctcatctcctgagagcggagcctcagccagcctaggtgcaggtcaccctcacacgtggccgcaccaaggccacctcacaatcacagccatgccacatgctgagcccccgagatgtgcaggccacatgccagctggctgcttctgttgtgtttctgcgggagctgcatgaggctgaaggagagacccgggcagagagaggctgagcctcttgctcttggaggcagacgcgggtttcggtgctgcccccctctgacaccctggtcgacatttctgttcctgcctcaggcctcctccttgaacctgcctctgacctcaccaggagtgggtttgtctggcgaccagaccctggtggccttctgctgaagcctctgcaatcgttccgcactgggggctcggtgaccgcaggtggaccctgctctgtgggaagcagcctgggggctacgggaggcagtgcgacgggtcaccccatctctctggagtttgacacagtctcccatctggcctcaactg

SEQ ID NO: 53 - DNA, 845 bp, Homo sapiens
  misc_feature: Incyte ID No 2771521CB1
ccgacgcgacccgcgccgcgtccgcggcggggagttgttgctgccgcgatgctggtggcggcggccgcggagcggaacaaggatcccatcttgcacgtgctgcggcagtacctggatccggcccagcgtggcgtccgcgtcctcgaggtggcctcgggctccggccagcacgcagcgcacttcgcgcgggccttccccctggccgagtggcagccgtcggacgtggaccagcgctgcctggacagcatcgcggccaccacgcaagcccagggcctgaccaacgtgaaggccccgctacacctggacgtgacgtggggctgggagcactggggcgggatcctgccacagtcgctggacctgttgctctgcatcaacatggcccatgtcagccccctgcgctgcacggaggggctcttcagagcagcaggacacctgctcaaacccagggccctgctcatcacctacgggccctatgccatcaatgggaagatctccccccagagcaacgtggactttgacctgatgctcagatgcaggaacccagaatgggggcttcgggacacagccctcctggaggacctgggaaaggccagtggcctgctcctggagaggatggtggacatgccagccaacaacaaatgcctgatcttccggaaaaactaagcccctccttcacccccgcacacctgcatccctgccggaggctctgtgaggcacgaaccctgcctccctaggccggaccttgtggacgacagccccacccagtctgtgctctcagccgctggccgaagggcccagcctgctcagaataaagcatgtcctgctgccggcaaaaaaaaaaaaaa

SEQ ID NO: 54 - DNA, 655 bp, Homo sapiens
  misc_feature: Incyte ID No 7095792CB1
gtaagttgcagacatgatatccctttacttaaaagtatttcagtgtgaattttctaagaacaagaatttcctcttatgtaaccacagtacattatcaaaatcaggaaatttgatgttggtgcaaaactgtgatctaatccttaaaccatattcagatttcatcaattgcctcaatgatagtctttatagctatttttcttatgctagtgtccagttcacaatcatgcctttcctttgcttgttgtgtctatttactctcttttaactttgaacagttcctcagcctttctttggtctcttttgccattgacagttttgaggaggccagttattttgttgaatgtccttcattctgggtttgtcttctgttttctcatgaccatatgcgggttaggcacttttggcaagcatattaccagaaatgatgatgcactttattggcatgtcatatcagggaatgacattgatgtgtctcattattggtgatcttgactgtgatcaattggtgatgacaaaggtgatggcttccaggttttccagtgtaaagttactgtttttctttttataactaataagcaatcatgaggcgatactttgaaactattgaaacatattgttcctcattaaactttcagttttagcatacattgacaac

SEQ ID NO: 55 - DNA, 1087 bp, Homo sapiens
  misc_feature: Incyte ID No 7112696CB1
tcactttcatcgtcaagaagggtgtccagctgaagctcatgtcgaaggcgccacgtcgctgacccaaaacaggggcggattcgctcctgcacctaacaacatatttggatacttcaatcatgaattcgatttcagcggtgaatgctttccgatttttggccctggcaacagtggccgcggccatgaccggctgctcgtttttgcaagttggtgagtctgagtactcctgtaagggcatgccagacggtgtgacctgtatgtccgctcgcgatgtttatcagctgactgaaaacgagaattttcgccaagtagtggagcagaaccagtcggccaaggatcaggcgataaaagagggcaagtcgattgaggaggttctccccgcggtgcagccccatatcgcagctggcgagcgctatgtcgttccgaagccagcccgtaacccgattccgattcgctcgcaggccaccgtcatgcgcgtctgggtggctccatgggagtccgattcgggcgacctgaacgtgcctggattcatctacacggaaattgaaccgcgccgttgggaaattggtactccggcgcctaaaccgacaccttccattcgaccactggagtcgaggaagaacacttcttcacctgcaacataatcattccccaaccagcatgacctaggagatagaccatgcaaatgagcatgaccccaagccaagtccggcgcacgaaggacgtaatgaagttcggtgtggcactgatgctcaccgtcgcggcaagcaacgcactcgccggcaccggtggtgacagtttcgactcaatctgggtaaccctgacggactggatgcagggaaccctgggtcgggtggtggcaggctcaatggtcctggtcgggatcgtatccggcatcgtgcgtcagtcgatcatgtcgtttgctaccggcgttggcggcggcgttggcctgtacaacacgccgaccatcatcgagtcgatcatgaccgccaccctcccaggttgcgcactctctcacctaagcaacgagtactaacaaagccggccgtttgccggcttttggatcttcaaaccaggatcgggataa

SEQ ID NO: 56 - DNA, 2869 bp, Homo sapiens
  misc_feature: Incyte ID No 7759388CB1
aaaaacaggctggtgcagacagcagagctcacaaaggtgctggcccaggccctgactccctgaagagagatgaagaaaccgagaatcagagaggttcagatacttgctggaggtcacacagctagtctcacctcccacccctcctgccttcccactgcaccatggctccaggacccttctcctcggccctcctctcgccgccgcccgctgccctgccctttctgctgctgctctgggcgggggcatctcgtggccagccctgccccggccgctgcatctgccagaacgtggcgcccacactgacaatgctgtgcgccaagaccggcttgctctttgtgccgcccgccatcgaccggcgcgtggtggagctgcggctcaccgacaacttcatcgccgccgtgcgccgccgagacttcgccaacatgaccagcctggtgcacctcactctctcccggaacaccatcggccaggtggcagctggcgccttcgccgacctgcgtgccctccgggccctgcacctggacagcaaccgcctggcggaggtgcgcggcgaccagctccgcggcctgggcaacctccgccacctgatccttggaaacaaccagatccgccgggtggagtcggcggcctttgacgccttcctgtccaccgtggaggacctggatctgtcctacaacaacctggaggccctgccgtgggaggcggtgggccagatggtgaacctaaacaccctcacgctggaccacaacctcatcgaccacatcgcggaggggaccttcgtgcagcttcacaagctggtccgtctggacatgacctccaaccgcctgcataaactcccgcccgacgggctcttcctgaggtcgcagggcaccgggcccaagccgcccaccccgctgaccgtcagcttcggcggcaaccccctgcactgcaactgcgagctgctctggctgcggcggctgacccgcgaggacgacttagagacctgcgccacgcccgaacacctcaccgaccgctacttctggtccatccccgaggaggagttcctgtgtgagcccccgctgatcacacggcaggcggggggccgggccctggtggtggaaggccaggcggtgagcctgcgctgccgagcggtgggtgaccccgagccggtggtgcactgggtggcacctgatgggcggctgctggggaactccagccggacccgggtccggggggacgggacgctggatgtgaccatcaccaccttgagggacagtggcaccttcacttgtatcgcctccaatgctgctggggaagcgacggcgcccgtggaggtgtgcgtggtacctctgcctctgatggcacccccgccggctgccccgccgcctctcaccgagcccggctcctctgacatcgccacgccgggcagaccaggtgccaacgattctgcggctgagcgtcggctcgtggcagccgagctcacctcgaactccgtgctcatccgctggccagcccagaggcctgtgcccggaatacgcatgtaccaggttcagtacaacagttccgttgatgactccctcgtctacaggatgatcccgtccaccagtcagaccttcctggtgaatgacctggcggcgggccgtgcctacgacttgtgcgtgctggcggtctacgacgacggggccacagcgctgccggcaacgcgagtggtgggctgtgtacagttcaccaccgctggggatccggcgccctgccgcccgctgagggcccatttcttgggcggcaccatgatcatcgccatcgggggcgtcatcgtcgcctcggtcctcgtcttcatcgttctgctcatgatccgctataaggtgtatggcgacggggacagccgccgcgtcaagggctccaggtcgctcccgcgggtcagccacgtgtgctcgcagaccaacggcgcaggcacaggcgcggcacaggccccggccctgccggcccaggaccactacgaggcgctgcgcgaggtggagtcccaggctgcccccgccgtcgccgtcgaggccaaggccatggaggccgagacggcatccgcggagccggaggtggtccttggacgttctctgggcggctcggccacctcgctgtgcctgctgccatccgaggaaacttccggggaggagtctcgggccgcggtgggccctcgaaggagccgatccggcgccctggagccaccaacctcggcgccccctactctagctctagttcctgggggagccgcggcccggccgaggccgcagcagcgctattcgttcgacggggactacggggcactattccagagccacagttacccgcgccgcgcccggcggacaaagcgccaccggtccacgccgcacctggacggggctggagggggcgcggccggggaggatggagacctggggctgggctccgccagggcgtgcctggctttcaccagcaccgagtggatgctggagagtaccgtgtgagcggcgggcgggcgccgggacgcctgggtgccgcagaccaaacgcccagccgcacggacgctggggcgggactgggagaaagcgcagcgccaagacattggaccagagtggagacgcgcccttgtccccgggagggggcggggcagcctcgggctgcggctcgaggccacgcccccgtgcccagggcggggttcggggaccggctgccggcctcccttcccctatggactcctcgacccccctcctacccctcccctcgcgcgctcgcggacctcgctggagccggtgccttacacagcgaagcgcggggaggggcagggccccctgacactgcagcactgagacacgagccccctcccccagcccgtcacccggggccggggcgaggggcc

SEQ ID NO: 57 - DNA, 2798 bp, Homo sapiens
  misc_feature: Incyte ID No 8165414CB1
cctgagcagcgctcttcggttgcagtacccactggaaggacttaggcgctcgcgtggacaccgcaagcccctcagtagcctcggcccaagaggcctgctttccactcgctagccccgccgggggtccgtgtcctgtctcggtggccggacccggcccggagccccgagcagtagccggcgccatgtcggtggtgggcatagacctgggcttccagagctgctacgtcgctgtggcccgcgccggcggcatcgagactatcgttaatgagtatagcgaccgctgcacgccggcttgcatttcttttggtcctaagaatcgttcaattggagcagcagctaaaagccaggtaatttctaatgcaaagaacacagtccaaggatttaaaagattccatggccgagcattctctgatccatttgtggaggcagaaaaatctaaccttgcatatgatattgtgcagttgcctacaggattaacaggtataaaggtgacatatatggaggaagagcgaaattttaccactgagcaagtgactgccatgcttttgtccaaactgaaggagacagccgaaagtgttcttaagaagcctgtagttgactgtgttgtttcggttccttgtttctatactgatgcagaaagacgatcagtgatggatgcaacacagattgctggtcttaattgcttgcgattaatgaatgaaaccactgcagttgctcttgcatatggaatctataagcaggatcttcctgccttagaagagaaaccaagaaatgtagtttttgtagacatgggccactctgcttatcaagtttctgtatgtgcatttaatagaggaaaactgaaagttctggccactgcatttgacacgacattgggaggtagaaaatttgatgaagtgttagtaaatcacttctgtgaagaatttgggaagaaatacaagctagacattaagtccaaaatccgtgcattattacgactctctcaggagtgtgagaaactcaagaaattgatgagtgcaaatgcttcagatctccctttgagcattgaatgttttatgaatgatgttgatgtatctggaactatgaatagaggcaaatttctggagatgtgcaatgatctcttagctagagtggagccaccacttcgtagtgttttggaacaaaccaagttaaagaaagaagatatttatgcagtggagatagttggtggtgctacacgaatccctgcggtaaaagagaagatcagcaaatttttcggtaaagaacttagtacaacattaaatgctgatgaagctgtcactcgaggctgtgcattgcagtgtgccatcttatcgcctgctttcaaagtcagagaattttctatcactgatgtagtaccatatccaatatctctgagatggaattctccagctgaagaagggtcaagtgactgtgaagtcttttccaaaaatcatgctgctcctttctctaaagttcttacattttatagaaaggaacctttcactcttgaggcctactacagctctcctcaggatttgccctatccagatcctgctatagctcagttttcagttcagaaagtcactcctcagtctgatggctccagttcaaaagtgaaagtcaaagttcgagtaaatgtccatggcattttcagtgtgtccagtgcatctttagtggaggttcacaagtctgaggaaaatgaggagccaatggaaacagatcagaatgcaaaggaggaagagaagatgcaagtggaccaggaggaaccacatgttgaagagcaacagcagcagacaccagcagaaaataaggcagagtctgaagaaatggagacctctcaagctggatccaaggataaaaagatggaccaaccaccccaagccaagaaggcaaaagtgaagaccagtactgtggacctgccaatcgagaatcagctattatggcagatagacagagagatgctcaacttgtacattgaaaatgagggtaagatgatcatgcaggataaactggagaaggagcggaatgatgctaagaacgcagtggaggaatatgtgtatgaaatgagagacaagcttagtggtgaatatgagaagtttgtgagtgaagatgatcgtaacagttttactttgaaactggaagatactgaaaattggttgtatgaggatggagaagaccagccaaagcaagtttatgttgataagttggctgaattaaaaaatctaggtcaacctattaagatacgtttccaggaatctgaagaacgaccaaaattatttgaagaactagggaaacagatccaacagtatatgaaaataatcagctctttcaaaaacaaggaggaccagtatgatcatttggatgctgctgacatgacaaaggtagaaaaaagcacaaatgaagcaatggagtggatgaataacaagctaaatctgcagaacaagcagagtttgaccatggatccagttgtcaagtcaaaagagattgaagctaaaattaaggagctgacaagtacttgtagccctataatttcaaagcccaaacccaaagtggaacctccaaaagaggaacaaaaaaatgcagagcagaatggaccagtggatggccacggagacaacccaggccccaggctgctgagcaggggtacagaacacagctgtgacttcggattcagcacaagaaagcttcctgaaatggacattgattgattccagcacttgctactattaaaacagactataataaagcttaaaagctggtaaatgcgatctaaatatcacacctcgcgcaagttgcat

SEQ ID NO: 58 - DNA, 3808 bp, Homo sapiens
  misc_feature: Incyte ID No 2540610CB1
ctgagaagttttcgcgggtttcagaagtttccttaggcgttctaagggctttactcaggtggagtctccattcaggcacttatttaaccacccatttctcctttaggggtcctcgctgctcgcccagccgctaattaagtgacggacacagtagctaaggagactgcctgattgacacgcatgattaccaaccgacttccggaaactccagtcagggcctgcccggcgcgtggcccacggcccaattaaagaagtggaagcgccaaagggggaggtaacggagcggcctacgttgtggcggttccctggtgaatgcgccctggggttgaggcgtctgcgggcgttcggacgatgccgtgacgcggcacggcgacactgttggcaatatgagcgcacccctgtagagggagcccttcggtcctggaggcggcgcggcgtgaagacaggttgctatttgagagcgttcccttgaagcccctcagagagtgggggaggggcggcggacggcaagcggttcctgtctgcgcttgcgccggcgcctctgccgacccggcctgcacgcacgcgcatgcccgtagcgcgcggagccgcggtggccggcagcactgcgcgtgcgcggtgaggagcccgctaaggagcggcgctggcggacgtcgggctggctgcccgtgacgtcgtgcggagagctttaaagtgcgggccgggccgggcgtccgagggtctggtcgggagtcgggccgcgtctccgcagcagccctccgcggcatgaggcgctgctggcgcccctgccccgcgggacgtggagaaggtggaggaggaagaagccccgttgtcgccaccgttgcatgacccgccgctcctgaggccctaccccacgcccggaccctcgacgccccccgccgggtcccccactcacgcatgggggttcggcgctaaggacccccctccctccgggggccccggggcgcgtccccttagagccatgcccggctgccccgcccgccccggaggaccctagagcagcgtcgtgggggccatggcggccgccagcggctacacggacctgcgtgagaagctcaagtccatgacgtcccgggacaactataaggcgggcagccgggaggccgccgccgctgccgcagccgccgtagccgccgcagccgcagccgccgctgccgccgaaccttaccctgtgtccggggccaagcgcaagtatcaggaggactcggaccccgagcgcagcgactatgaggagcagcagctgcagaaggaggaggaggcgcgcaaggtgaagagcggcatccgccagatgcgcctcttcagccaggacgagtgcgccaagatcgaggcccgcattgacgaggtggtgtcccgcgctgagaagggcctgtacaacgagcacacggtggaccgggccccactgcgcaacaagtacttcttcggcgaaggctacacttacggcgcccagctgcagaagcgcgggcccggccaggagcgcctctacccgccgggcgacgtggacgagatccccgagtgggtgcaccagctggtgatccaaaagctggtggagcaccgcgtcatccccgagggcttcgtcaacagcgccgtcatcaacgactaccagcccggcggctgcatcgtgtctcacgtggaccccatccacatcttcgagcgccccatcgtgtccgtgtccttctttagcgactctgcgctgtgcttcggctgcaagttccagttcaagcctattcgggtgtcggaaccagtgctttccctgccggtgcgcaggggaagcgtgactgtgctcagtggatatgctgctgatgaaatcactcactgcatacggcctcaggacatcaaggagcgccgagcagtcatcatcctcaggaagacaagattagatgcaccccggttggaaacaaagtccctgagcagctccgtgttaccacccagctatgcttcagatcgcctgtcaggaaacaacagggaccctgctctgaaacccaagcggtcccaccgcaaggcagaccctgatgctgcccacaggccacggatcctggagatggacaaggaagagaaccggcgctcggtgctgctgcccacacaccggcggaggggtagcttcagctctgagaactactggcgcaagtcatacgagtcctcagaggactgctctgaggcagcaggcagccctgcccgaaaggtgaagatgcggcggcactgagtctacccgccgccctcctgggaactctggctcatccttacgtagttgcccctccttttgttttgagggttttgtttttgttcattggggggtttttgttttttgttttttgttttttttgattctatatatttttccttggttttgttgcctgttagggctgaagaatagaattggccaggacctaggttctcatattcttggtattcctcctggatggaaaggctgttggcatcaataggggacagaggctgatgctggagtggccagtagaggtggtggagcagagcagccatcttttaagtggggctgtatcaggctgggtttatttaaaagcaacaaaatgttttggttaagaaaattattttgctttcagtgtaaatcttcgcagtgttctaaacaaagttcagtcttctgctcgcccctttccctcactgatgtctgcacttggttgaggtctcctggagcctcacaggctctgctgttctccacttctcacctgccatccacgccctgcaagctcatgcaaacaccctttcttcctcctgcggcagagttgttcaggttgcctgggcaggggcttaaacagtgccagcccctgccatcccaaagctattgttaagccccccaggcgtcctccacccacgcccactagcctgccatgtccacagttccttgggctgctgaggggctagtgcagtggtcctgacctctcttatcaagagcacacttctttgctggttgctccttttgagcatatgcgtgtgattatttggaacagttagacttgccacgttgggtcagttttagaaattgtttctagctagagggactggtgtccttccaagtctagcatttggggtatggaaaattgttgtggtgtgtggtagggtttttgttttcttttttgagttttttttccccctttagtctcctggctttttcctttcccttcccttctccactggccagcttgggcctcatcctcatgtcatccttctaggaaggcgcctgccccatcttgtctgccggcagcatgcatccaaggccagagctcaggcctgcagactgggctggtgcctcctccgcttcagggtatgggagttggtgaaggggctttcaaaaaataataaggaaaaaaaggtaaagtctttggtagcttctatccactcagatcctggaaggcagcaaggttttgtggatctagattcattaggaatgtcttcttgtcagccaggccaggacccgggcttgccaagagcagaggccctcccagcaaccaggataccaccactttgggggctttgtgtacagaggtccgggtctgagacctcataggctgcagaaatctggggcagccaccatcaagaagcccctctcaggggccagaactcctttgccagcgtggatttctcaagtcgggactgcataattaaagcagttgcagttttattttttttacagcttttttcccaaaaatgatttgtagttgtgtgtgcagcacttcgccctgatatgtgtgctctacaataaaaaccaaatctaatatattttgaaaaaaaaagggtacccaaaaaaaaaaaaaaa

SEQ ID NO: 59 - DNA, 1877 bp, Homo sapiens
  misc_feature: Incyte ID No 1593380CB1
tgcgcaagcatgccataccctgtacttccagcttctgcaccctagaaccaagcagcagccacttgcctgggggtagagtcgggggcagcaagcgacacacatccacgtcctgagcaggaagctcctgacctgctgggcatgggtggagaaggagaaggtggcagggttgggtggctctccctacatggtcacatcctgtgaagacactgaggacaccaggaattctaaatttgaacctgatgccccagagctttaagaaaatgtttttgtcaaaggagaaaaccagaacgttttattagtttcttgaacctatttatagcgtcaaaagttagacgcaggtgcaggcctccattccatctattggctggctctcgactgccgagactggcctgccaacctgtgtttcaggagggcacgcgtctgcggctgaaccgcggaagggccggtgaggaaccgggcctcgggagatggccctgagggcccccgcactgctgccgctgctgctgctactactgccgctccgcgccgccggctgcccagcagcctgccgctgctacagcgccacggtggagtgtggcgccctgcggttgcgcgtcgtcccgctgggaatcccgccagggacgcaggtgggcaccgtgtggagctgcgggaggacggggtgcccccagggagaggaagacccccgcaacggggtaagcgcctcctctttccccggcctgcagacactgttcctgcaggacaacaacatcgcccgcctagagccgggagccctggcgccactcgccgctctgcgccggctctacctgcacaacaacagcctgcgcgccctggaggccggcgccttccgcgcgcagccgcgcctgctggagctggcgctcactagcaaccggctgcgcggcttgcgcagcggcgccttcgtaggcctggcccagctgcgcgtgctctacctggcgggcaaccagctggcgcggctgctggatttcaccttcttgcacctgccggtgagcgcctggggtctaaaggggcgggatactccattatggcccctcgccctgtagggctggaatagttagaaaaggcaacccagtctagcttggtaagaagagagacatgcccccaacctcggcgccctttttcctcacgatctgctgtccttacttcagcgactgcaggagcttcacctgcaagaaaacagcattgagctgctggaggaccaggctctagcggggctgtcctccctagcactgctggacctcagcaggaaccagctgggcaccatcagccgagaggccctgcagcccctggccagtctgcaagtcctgcgcctcacaggtacctcttcctcggaaagcgtctctgtctgggccacggtgttggcagtagggagcaggctcatgtgtggaggggctctgacatcaggcaactgggcagcagtctggaaggctgcaccaccgcctgcagccacactggagggctgcgggagtgggtgtgctggggaatacagctcaggcaccctgcctggggaaaacagaagacaggaactccacagggtgacttaacagaattgagggttctgcacacttttaggctgaggggaggcagacaaaattgcaaaggagagagacagctgaccaggactggggacattcccaagagagggcatccaaaggcaaggctgagaggacttgtgaggtttcagtgctggtgagatgtctatgtggacgcccaggttcctaaaaacagtggcacttgttgcaggaagacttaaggagcaggtgctttctcagagaccgacggagaagtgatctgtaacaggcaggcatcacagagctaaaggggaggggggcgagggtgaagggcctgccgccccaaggagc

SEQ ID NO: 60 - DNA, 1688 bp, Homo sapiens
  misc_feature: Incyte ID No 1480069CB1
cagagtctcgctcttgttgtccaggctagagtgcaatgaagcgtttttgggctcaccccacacttaggcttccgggggctcaagggattcttcctggctttagcctcccaaagtaggtgggattacaagcacgtgccaccatgtctgcctaattttgtatttttagtagagatggggtttcttcatgttggtcaggctggtctccaactccccacctcaggtgatccacccgcctcagcctcccaaagtgccgggattacaggcgtgagccaccgtgcccagcaacagagattctagtggggcccattttaacagaaggggcactggaaatgaatgtgtccagaagaaggtaactggaaagcaaattgcaggaatgagagaaggaactgcttagtctgggaaagcctaagttatttgcagaattgaattgaaaagatttccggggagaacagatagctagcatcctacatctgtagagcttttacagaacaatgttaagttgcctcctaacgagctttagcatggcaccagtggaaggcactgttagatggcagcaatgcagagttgtggaaaggccaaggggcagaagatttgaggcagatcagctcctatctgtgaagcagtatagtgggaggagcacagactttggagtcacacagacctgggttctagtcccagcctacctctaccagctatgtgaccttggggaagttgctgaacctctttgtgcctcagtttccatatgtacaaaataaagaggctagattggaagacttccaagatcttgtctagctataaaattccttggttctaaaaaggtggcttatgtgcataaaataaatagatggaaatacagtattatcttctttggattttgctctcaaatctgtactaatattggttatcaagtatgcacagccagcctgcttctgttgcactgcatgatggcatgtaatgatggaacagagctgacatgttcacaaaagcacttttctctgtgggtaggagtggaaaatagttccatttagctgatatcactaatttttaaagaaaaccatatgagctaaagagaactgaagatgaatgtgggagctatgagtcaaccactgaatctcagctgcattaattttacattataaatgacacagttttaaagtttcttagattttataatagttgaaatttttctttaaaagagtacaccctatacatgccgatacacgttaaaaagattgaacatttcttcccattctaaagcatcttgaaaacaatgactgtgctgttagcgatttttaatataaaatacattttctttcaagtgggtttagtaccgcgctaaacagcactttttatagaaaattgggctaacttcttacaaaaataaacctgcacaattttaatgttttcttttgtatgtaattttcctaccccacttaatttgaatgtgttttgataattaggataaatatgtcgcttaaaaaaaacatccaggcctattcccccctaacaatcctcaaaaaagagaaatgtcaatcatattctaatgaatgattaaatgtggtagtataagctatattaaaataagataaaattgttggggacgccgaaggctttattcccccattgaggtgggagggtataaatttgtgatcattgagaaccagggcacgattgttttgtaacaaaag

SEQ ID NO: 61 - DNA, 776 bp, Homo sapiens
  misc_feature: Incyte ID No 2310442CB1
tacgggaagcagcactggtggtgcctcagccatggcctggaccgttctcctcctcggcctcctctctcactgcacaggctatgtgacctcctatgtgctgactcagccaccctcggtgtcagtggccccaggaaagacggccaggatttcctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattcagtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctaaaccctcacccccccccacgggagactagagctgcag

SEQ ID NO: 62 - DNA, 3158 bp, Homo sapiens
  misc_feature: Incyte ID No 7503731CB1
ctgagaagttttcgcgggtttcagaagtttccttaggcgttctaagggctttactcaggtggagtctccattcaggcacttatttaaccacccatttctcctttaggggtcctcgctgctcgcccagccgctaattaagtgacggacacagtagctaaggagactgcctgattgacacgcatgattaccaaccgacttccggaaactccagtcagggcctgcccggcgcgtggcccacggcccaattaaagaagtggaagcgccaaagggggaggtaacggagcggcctacgttgtggcggttccctggtgaatgcgccctggggttgaggcgtctgcgggcgttcggacgatgccgtgacgcggcacggcgacactgttggcaatatgagcgcacccctgtagagggagcccttcggtcctggaggcggcgcggcgtgaagacaggttgctatttgagagcgttcccttgaagcccctcagagagtgggggaggggcggcggacggcaagcggttcctgtctgcgcttgcgccggcgcctctgccgacccggcctgcacgcacgcgcatgcccgtagcgcgcggagccgcggtggccggcagcactgcgcgtgcgcggtgaggagcccgctaaggagcggcgctggcggacgtcgggctggctgcccgtgacgtcgtgcggagagctttaaagtgcgggccgggccgggcgtccgagggtctggtcgggagtcgggccgcgtctccgcagcagccctccgcggcatgaggcgctgctggcgcccctgccccgcgggacgtggagaaggtggaggaggaagaagccccgttgtcgccaccgttgcatgacccgccgctcctgaggccctaccccacgcccggaccctcgacgccccccgccgggtcccccactcacgcatgggggttcggcgctaaggacccccctccctccgggggccccggggcgcgtccccttagagccatgcccggctgccccgcccgccccggaggaccctagagcagcgtcgtgggggccatggcggccgccagcggctacacggacctgcgtgagaagctcaagtccatgacgtcccgggacaactataaggcgggcagccgggaggccgccgccgctgccgcagccgccgtagccgccgcagccgcagccgccgctgccgccgaaccttaccctgtgtccggggccaagcgcaagtatcaggaggactcggaccccgagcgcagcgactatgaggagcagcagctgcagaaggaggaggaggcgcgcaaggtgaagagcggcatccgccagatgcgcctcttcagccaggacgagtgcgccaagatcgaggcccgcattgacgaggtggtgtcccgcgctgagaagggcctgtacaacgagcacacggtggaccgggccccactgcgcaacaagtacttcttcggcgaaggctacacttacggcgcccagctgcagaagcgcgggcccggccaggagcgcctctacccgccgggcgacgtggacgagatccccgagtgggtgcaccagctggtgatccaaaagctggtggagcaccgcgtcatccccgagggcttcgtcaacagcgccgtcatcaacgactaccagcccggcggctgcatcgtgtctcacgtggaccccatccacatcttcgagcgccccatcgtgtccgtgtccttctttagcgactctgcgctgtgcttcggctgcaagttccagttcaagcctattcgggtgtcggaaccagtgctttccctgccggtgcgcaggggaagcgtgactgtgctcagtagaggtggtggagcagagcagccatcttttaagtggggctgtatcaggctgggtttatttaaaagcaacaaaatgttttggttaagaaaattattttgctttcagtgtaaatcttcgcagtgttctaaacaaagttcagtcttctgctcgcccctttccctcactgatgtctgcacttggttgaggtctcctggagcctcacaggctctgctgttctccacttctcacctgccatccacgccctgcaagctcatgcaaacaccctttcttcctcctgcggcagagttgttcaggttgcctgggcaggggcttaaacagtgccagcccctgccatcccaaagctattgttaagccccccaggcgtcctccacccacgcccactagcctgccatgtccacagttccttgggctgctgaggggctagtgcagtggtcctgacctctcttatcaagagcacacttctttgctggttgctccttttgagcatatgcgtgtgattatttggaacagttagacttgccacgttgggtcagttttagaaattgtttctagctagagggactggtgtccttccaagtctagcatttggggtatggaaaattgttgtggtgtgtggtagggtttttgttttcttttttgagttttttttccccctttagtctcctggctttttcctttcccttcccttctccactggccagcttgggcctcatcctcatgtcatccttctaggaaggcgcctgccccatcttgtctgccggcagcatgcatccaaggccagagctcaggcctgcagactgggctggtgcctcctccgcttcagggtatgggagttggtgaaggggctttcaaaaaataataagaaaaaaaaggtaaagtctttggtagcttctatccactcagatcctggaaggcagcaaggttttgtggatctagattcattaggaatgtcttcttgtcagccaggccaggacccgggcttgccaagagcagaggccctcccagcaaccaggataccaccactttgggggctttgtgtacagaggtccgggtctgagacctcataggctgcagaaatctggggcagccaccatcaagaagcccctctcaggggccagaactcctttgccagcgtggatttctcaagtcgggactgcataattaaagcagttgcagttttattttttttacagcttttttcccaaaaatgatttgtagttgtgtgtgcagcacttcgccctgatatgtgtgctctacaataaaaaccaaatctaatatattttgaaaaaaaaagggtacccaaaaaaaaaaaaaaaaaaaaaaaaaaaaaaaaaaaaaaaaaaagggaggc

SEQ ID NO: 63 - DNA, 3024 bp, Homo sapiens
  misc_feature: Incyte ID No 7506368CB1
ggcggaagaggtgctgtgcaggaggcggaagaggtgctgtgcaggaggcgggcgggcgcggttctttccggaaggattgaatctcctttagccccgcccgcctccgtagctgcctgaagtagtgcagggtcagcccgcaagttgcaggtcatggcgctggctgctcgactgtggcgccttctgcctttccgacgtggagccgccccggggtctcgtctccctgcggggacttcgggcagccgcgggcattgcggcccctgtcgattccgcggcttcgaggtaatgggaaacccaggaactttcaacagaggccttttactctcagctttgtcgtatttgggttttgaaacttaccaggttatctctcaggctgctgtggttcatgccacagccaaagttgaagaaatacttgaacaagcagactacctgtatgaaagcggagaaacagaaaaactttatcagttgctaacccaatacaaggaaagtgaagatgcagagttactgtggcgtttggcacgggcatcacgtgatgtagctcagcttagcagaacctcagaagaggagaaaaagctattggtgtatgaagccctagagtatgcaaaaagagcactagaaaaaaatgaatcaagttttgcatctcataagtggtatgcaatctgtcttagtgatgttggagattatgaaggcatcaaggctaaaattgcaaatgcatatatcatcaaggagcattttgagaaagcaattgaactgaaccctaaagatgctacttcaattcaccttatgggtatttggtgctatacatttgccgaaatgccttggtatcaaagaagaattgctaaaatgctgtttgcaactcctcctagttccacctatgagaaggccttaggctactttcacagggcagaacaaggaaagacatacttgaaactacacaacaaaaagcttgctgctttctggctaatgaaagccaaggactatccagcacacacagaggaggataaacagatacagacagaagctgctcagttgcttacaagtttcagtgagaagaattgagaacttttcagagaagatttatgaaatagctaataaacattgccttttcttttaattctaaacttaatatatgaactataactgttctacggctttttaaatgttgtgaccatttaaccgtgtaaatataaaatattctaggcttcttcacaaataatagggtaaaataaataatcgccataagagtggtagaaataaatctccatggctcaggcaaagagattattttgcatcctggataccagcaatgcaaaatggtatgagatttctaaggattgatcacattgggatgggagatcaagcaaagaaatatttgtagaggaggggaaatggatctataggggatatacagggggatggattttcaaattggattgattctaagttgaaatcttgaagagaaggtgtggtgacagtggttaggatgttgtgggttcctgacataaagtagttaaatgatatatcttggagctaacctgtgtaagtaaagaactaagtaaggagatgactaaaaatggagtagtttccttttttatttttttgagacagagtctcactttgtttcccaggctggtgtgcagtggcacaatctcggcccactgcagcctccgcctcccgggttcaagtgattctcctgccttagcctcctgagtggctgggattacagggttgtaccaccacactcggctaacttttgtatttttggtagagatggggttttgccatgttggctaggctggtctcaaactcctggcctcaagtgatctgcccgccttggcctcccaaattgctgggattacaggcgtgagccaccgcacctggccagtttactttaaatgtggtgtagtctcatggtaaactgaatttgtcatcagatgcaaagttctattccctaatggaatggaaggaacacaaaacttaagagtgaaatggaatactaagatgtttttaaataggcaggactatgctactcacttgaggctggagtgccaccactgcaaaatctttttaagttttgtaaaaaggagcatcttgaatccacttagataaagacagactgtgtgtgtaggtggatttttcccaaaggatttgggaattgtaatgttacaatgaactgtatggatatgtttgtcatgtacattttcaaacaaaaaggaaaactgaaagtagtgatctttgtatacccatctcttagattcagtgattttgctatataggttgtgtatcccttatctgaaatacttgggactagtagaagcatcttggatttgggatgtttttccaaattttggaatacctgcatacacacaataagatatcttggagatgggacccaagtttaaacacaaattcacttgtttcatatataccttatgcacatagcttgaaggtaactttatataacattatttttaataattttgtgcattgagaccaagtttgcataccttgaaccatcagaaagcaaaggtgtcattatctcagccactcatgtgggtaatttgtggttggttgatgtcaccatcattcctgactgaatgtatatgctaccaataagcagttattttcttatacttattcatgcataagtacttaacagtaaaaaatatgacataactcgcacaggaacaaggatggcaaaaaaaaaaatatgacacaccactgatacagtgaaaaaataatgtggtcagggtagctaggcaacagtagcatcaccagaaacctgtatcagctgttaaacggcaacaacaatggcaggctttcagtttcccacttaatgatgctgtattttaaaaggttattgtatactgtaattttatttttgtaggtgaagagaaacagaagcagctgaagggccaggaagtgggtctttctagggatgtggcattctgctggatggctttttaaaatgggttttttcctttagggagaccgaataaactgtgttgtgcacctgcaaaaaaaaaa

SEQ ID NO: 64 - DNA, 948 bp, Homo sapiens
  misc_feature: Incyte ID No 7509087CB1
ccgacgcgacccgcgccgcgtccgcggcggggagttgttgctgccgcgatgctggtggcggcggccgcggagcggaacaaggatcccatcttgcacgtgctgcggcagtacctggatccggcccagcgtggcgtccgcgtcctcgaggtggcctcgggctccggccagcacgcagcgcacttcgcgcgggccttccccctggccgagtggcagccgtcggacgtggaccagcgctgcctggacagcatcgcggccaccacgcaagcccagggcctgaccaacgtgaaggccccgctacacctggacgtgacgtggggctgggagcactggggcgggatcctgccacagtcgctggacctgttgctctgcatcaacatggcccatgtcagccccctgcgctgcacggaggggctcttcagagcagcaggacacctgctcaaacccagggccctgctcatcacctacgggggagggggtgccctttcattctccaaaccccagcctgcaagtctgtcccaggctctctgggaaataccccgaccttggtccagtgggctgtgtccccatgcccacagccctatgccatcaatgggaagatctccccccagagcaacgtggactttgacctgatgctcagatgcaggaacccagaatgggggcttcgggacacagccctcctggaggacctgggaaaggccagtggcctgctcctggagaggatggtggacatgccagccaacaacaaatgcctgatcttccggaaaaactaagcccctccttcacccccgcacacctgcatccctgccggaggctctgtgaggcacgaaccctgcctccctaggccggaccttgtggacgacagccccacccagtctgtgctctcagccgctggccgaagggcccagcctgctcagaataaagcatgtcctgctgccggcaaaaaaaaaa

(19) United States
(12) Patent Application Publication
(10) Pub. No.: US 2004/0158039 A1
(43) Pub. Date: Aug. 12, 2004

(54) Secreted proteins

(76) Inventors: Henry Yue, Sunnyvale, CA (US); Ernestine A. Lee, Kensington, CA (US); Shanya D Becha, San Francisco, CA (US); Mariah R Baughn, Los Angeles, CA (US); Monique G Yao, Mountain View, CA (US); Y Tom Tang, San Jose, CA (US); Janice K Au-Young, Brisbane, CA (US); Preeti G Lal, Santa Clara, CA (US); Bridget A Warren, San Marcos, CA (US); Brendan M Duggan, Sunnyvale, CA (US); Uyen K Tran, San Jose, CA (US); Kavitha Thangavelu, Sunnyvale, CA (US); Thomas W Richardson, Redwood City, CA (US); Olga Bandman, Mountain View, CA (US); Karen A Jones, Bollington (GB); Junming Yang, San Jose, CA (US); Brooke M Emerling, Chicago, IL (US); Anita Swarnakar, San Francisco, CA (US); Wen Luo, San Diego, CA (US); Narinder K Chawla, Union City, CA (US); Yalda Azimzai, Oakland, CA (US); Farrah A Khan, Des Plaines, IL (US); Dyung Aina M Lu, San Jose, CA (US); Jennifer A Griffin, Fremont, CA (US); Soo Yeun Lee, Mountain View, CA (US); Neil Burford, Durham, CT (US); Vicki S Elliott, San Jose, CA (US); Cynthia D Honchell, San Francisco, CA (US); Ann He, San Jose, CA (US); Patricia M Mason, Morgan Hill, CA (US); Joana X Li, Millbrae, CA (US); April JA Hafalia, Daly City, CA (US); Rajagopal Gururajan, San Jose, CA (US)

Correspondence Address: FOLEY AND LARDNER, SUITE 500, 3000 K STREET NW, WASHINGTON, DC, 20007, US

(21) Appl. No.: 10/479,284

(22) PCT Filed: May 21, 2002
(86) PCT No.: PCT/US02/16234

(51) Int. Cl. C07K 14/705; C12N 5/06; C07H 21/04

(52) U.S. Cl. 530/350; 435/69.1; 435/320.1; 435/325; 536/23.5

(57) ABSTRACT

The invention provides human secreted proteins (SECP) and polynucleotides which identify and encode SECP. The invention also provides expression vectors, host cells, antibodies, agonists, and antagonists. The invention also provides methods for diagnosing, treating, or preventing disorders associated with aberrant expression of SECP.

### TECHNICAL FIELD

This invention relates to nucleic acid and amino acid sequences of secreted proteins and to the use of these sequences in the diagnosis, treatment, and prevention of cell proliferative, autoimmune/inflammatory, cardiovascular, neurological, and developmental disorders, and in the assessment of the effects of exogenous compounds on the expression of nucleic acid and amino acid sequences of secreted proteins.

### BACKGROUND OF THE INVENTION

Protein transport and secretion are essential for cellular function. Protein transport is mediated by a signal peptide located at the amino terminus of the protein to be transported or secreted. The signal peptide is comprised of about ten to twenty hydrophobic amino acids which target the nascent protein from the ribosome to a particular membrane bound compartment such as the endoplasmic reticulum (ER). Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Proteins that are retained in the plasma membrane contain one or more transmembrane domains, each comprised of about 20 hydrophobic amino acid residues. Secreted proteins are generally synthesized as inactive precursors that are activated by post-translational processing events during transit through the secretory pathway. Such events include glycosylation, proteolysis, and removal of the signal peptide by a signal peptidase. Other events that may occur during protein transport include chaperone-dependent unfolding and folding of the nascent protein and interaction of the protein with a receptor or pore complex. Examples of secreted proteins with amino terminal signal peptides are discussed below and include proteins with important roles in cell-to-cell signaling. Such proteins include transmembrane receptors and cell surface markers, extracellular matrix molecules, cytokines, hormones, growth and differentiation factors, enzymes, neuropeptides, vasomediators, cell surface markers, and antigen recognition molecules. (Reviewed in Alberts, B. et al. (1994) Molecular Biology of The Cell, Garland Publishing, New York, N.Y., pp. 557-560, 582-592.)

Cell surface markers include cell surface antigens identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocyte Antigen Facts Book, Academic Press, San Diego, Calif., pp. 17-20.)

Matrix proteins (MPs) are transmembrane and extracellular proteins which function in formation, growth, remodeling, and maintenance of tissues and as important mediators and regulators of the inflammatory response. The expression and balance of MPs may be perturbed by biochemical changes that result from congenital, epigenetic, or infectious diseases. In addition, MPs affect leukocyte migration, proliferation, differentiation, and activation in the immune response. MPs are frequently characterized by the presence of one or more domains which may include collagen-like domains, EGF-like domains, immunoglobulin-like domains, and fibronectin-like domains. In addition, MPs may be heavily glycosylated and may contain an Arginine-Glycine-Aspartate (RGD) tripeptide motif which may play a role in adhesive interactions. MPs include extracellular proteins such as fibronectin, collagen, galectin, vitronectin and its proteolytic derivative somatomedin B; and cell adhesion receptors such as cell adhesion molecules (CAMs), cadherins, and integrins. (Reviewed in Ayad, S. et al. (1994) The Extracellular Matrix Facts Book, Academic Press, San Diego, Calif., pp. 2-16; Ruoslahti, E. (1997) Kidney Int. 51:1413-1417; Sjaastad, M. D. and Nelson, W. J. (1997) BioEssays 19:47-55.)

Mucins are highly glycosylated glycoproteins that are the major structural component of the mucus gel. The physiological functions of mucins are cytoprotection, mechanical protection, maintenance of viscosity in secretions, and cellular recognition. MUC6 is a human gastric mucin that is also found in gall bladder, pancreas, seminal vesicles, and female reproductive tract (Toribara, N. W. et al. (1997) J. Biol. Chem. 272:16398-16403). The MUC6 gene has been mapped to human chromosome 11 (Toribara, N. W. et al. (1993) J. Biol. Chem. 268:5879-5885). Hemomucin is a novel Drosophila surface mucin that may be involved in the induction of antibacterial effector molecules (Theopold, U. et al. (1996) J. Biol. Chem. 217:12708-12715).

Tuftelins are one of four different enamel matrix proteins that have been identified so far. The other three known enamel matrix proteins are the amelogenins, enamelin and ameloblastin. Assembly of the enamel extracellular matrix from these component proteins is believed to be critical in producing a matrix competent to undergo mineral replacement. (Paine C. T. et al. (1998) Connect Tissue Res. 38:257-267). Tuftelin mRNA has been found to be expressed in human ameloblastoma tumor, a non-mineralized odontogenic tumor (Deutsch D. et al. (1998) Connect Tissue Res. 39:177-184).

Olfactomedin-related proteins are extracellular matrix, secreted glycoproteins with conserved C-terminal motifs. They are expressed in a wide variety of tissues and in broad range of species, from Caenorhabditis elegans to Homo sapiens. Olfactomedin-related proteins comprise a gene family with at least 5 family members in humans. One of the five, TIGR/myocilin protein, is expressed in the eye and is associated with the pathogenesis of glaucoma (Kulkarni, N. H. et al., (2000) Genet. Res. 76:41-50). Research by Yokoyama et al. (1996) found a 135-amino acid protein, termed AMY, having 96% sequence identity with rat neuronal olfactomedin-releated ER localized protein in a neuroblastoma cell line cDNA library, suggesting an essential role for AMY in nerve tissue (Yokoyama, M. et al., (1996) DNA Res. 3:311-320). Neuron-specific olfactomedin-related glycoproteins isolated from rat brain cDNA libraries show strong sequence similarity with olfactomedin. This similarity is suggestive of a matrix-related function of these glycoproteins in neurons and neurosecretory cells (Danielson, P. E. et al., (1994) J. Neurosci. Res. 38:468478).

Mac-2 binding protein is a 90-kD serum protein (90K) and another secreted glycoprotein, isolated from both the human breast carcinoma cell line SK-BR-3, and human breast milk. It specifically binds to a human macrophage-associated lectin, Mac-2. Structurally, the mature protein is 567 amino acids in length and is proceeded by an 18-amino acid leader. There are 16 cysteines and seven potential N-linked glycosylation sites. The first 106 amino acids represent a domain very similar to an ancient protein superfamily defined by a macrophage scavenger receptor cysteine-rich domain (Koths, K. et al., (1993) J. Biol. Chem. 268:14245-14249). 90K is elevated in the serum of subpopulations of AIDS patients and is expressed at varying levels in primary tumor samples and tumor cell lines. Ulhrich et al. (1994) have demonstrated that 90K stimulates host defense systems and can induce interleukin-2 secretion. This immune stimulation is proposed to be a result of oncogenic transformation, viral infection or pathogenic invasion (Ullrich, A., et al. (1994) J. Biol. Chem. 269:18401-18407).

Semaphorins are a large group of axonal guidance molecules consisting of at least 30 different members and are found in vertebrates, invertebrates, and even certain viruses. All semaphorins contain the sema domain which is approximately 500 amino acids in length. Neuropilin, a sernaphorin receptor has been shown to promote neurite outgrowth in vitro. The extracellular region of neuropilins consists of three different domains: CUB, discoidin, and MAM domains. The CUB and the MAM motifs of neuropilin have been suggested as having roles in protein-protein interactions and are suggested to be involved in the binding of semaphorins through the sema and the C-terminal domains (reviewed in Raper, J. A. (2000) Curr. Opin. Neurobiol. 10:88-94). Plexins are neuronal cell surface molecules that mediate cell adhesion via a homophilic binding mechanism in the presence of calcium ions. Plexins have been shown to be expressed in the receptors and neurons of particular sensory systems (Ohta, K. et al. (1995) Cell 14:1189-1199). There is evidence that suggests that some plexins function to control motor and CNS axon guidance in the developing nervous system. Plexins, which themselves contain complete semaphorin domains, may be both the ancestors of classical semaphorins and binding partners for semaphorins (Winberg, M. L. et al (1998) Cell 95:903-916).

Human pregnancy-specific beta 1-glycoprotein (PSG) is a family of closely related glycoproteins of molecular weights of 72 KDa, 64 KDa, 62 KDa, and 54 KDa. Together with the carcinoembryonic antigen, they comprise a subfamily within the immunoglobulin superfamily (Plouzek C. A. and Chou J. Y., Endocrinology 129:950-958) Different subpopulations of PSG have been found to be produced by the trophoblasts of the human placenta, and the amnionic, and chorionic membranes (Plouzek C. A. et al. (1993) Placenta 14:277-285).

Autocrine motility factor (AMF) is one of the motility cytokines regulating tumor cell migration, therefore identification of the signaling pathway coupled with it has critical importance. Autocrine motility factor receptor (AMFR) expression has been found to be associated with tumor progression in thymoma (Ohta Y. et al. (2000) Int. J. Oncol. 17:259-264). AMF is a cell surface glycoprotein of molecular weight 78 KDa.

Hormones are secreted molecules that travel through the circulation and bind to specific receptors on the surface of, or within, target cells. Although they have diverse biochemical compositions and mechanisms of action, hormones can be grouped into two categories. One category includes small lipophilic hormones that diffuse through the plasma membrane of target cells, bind to cytosolic or nuclear receptors, and form a complex that alters gene expression. Examples of these molecules include retinoic acid, thyroxine, and the cholesterol-derived steroid hormones such as progesterone, estrogen, testosterone, cortisol, and aldosterone. The second category includes hydrophilic hormones that function by binding to cell surface receptors that transduce signals across the plasma membrane. Examples of such hormones include amino acid derivatives such as catecholamines (epinephrine, norepinephrine) and histamine, and peptide hormones such as glucagon, insulin, gastrin, secretin, cholecystokinin, adrenocorticotropic hormone, follicle stimulating hormone, luteinizing hormone, thyroid stimulating hormone, and vasopressin. (See, for example, Lodish et al. (1995) Molecular Cell Biology, Scientific American Books Inc., New York, N.Y., pp. 856-864.)

Pro-opiomelanocortin (POMC) is the precursor polypeptide of corticotropin (ACH) a hormone synthesized by the anterior pituitary gland, which functions in the stimulation of the adrenal cortex. POMC is also the precursor polypeptide of the hormone, beta-lipotropin (beta-LPH). Each hormone includes smaller peptides with distinct biological activities: alpha-melanotropin (alpha-MSH) and corticotropin-like intermediate lobe peptide (CLIP) are formed from ACTH; gamma-lipotropin (gamma-LPH) and beta-endorphin are peptide components of beta-LPH, while beta-MSH is contained within garnua-LPH. Adrenal insufficiency due to ACTH deficiency, resulting from a genetic mutation in exons 2 and 3 of POMC results in an endocrine disorder characterized by early-onset obesity, adrenal insufficiency, and red hair pigmentation (Chretien, M. et al., (1979) Canad. J. Biochem. 57:1111-1121, Krude, H. et al., (1998) Nature Genet. 19:155-157, Online Mendelian Inheritance in Man, OMIM. Johns Hopkins University, Baltimore, Md. OMIM Number: 176830: Aug. 1, 2000. World Wide Web URL: www.ncbi.nlm.nih.gov/omim/).

Growth and differentiation factors are secreted proteins which function in intercellular communication. Some factors require oligomerization or association with membrane proteins for activity. Complex interactions among these factors and their receptors trigger intracellular signal transduction pathways that stimulate or inhibit cell division, cell differentiation, cell signaling, and cell motility. Most growth and differentiation factors act on cells in their local environment (paracrine signaling). There are three broad classes of growth and differentiation factors. The first class includes the large polypeptide growth factors such as epidermal growth factor, fibroblast growth factor, transforming growth factor, insulin-like growth factor, and platelet-derived growth factor. The second class includes the hematopoietic growth factors such as the colony stimulating factors (CSFs). Hematopoietic growth factors stimulate the proliferation and differentiation of blood cells such as B-lymphocytes, T-lymphocytes, erythrocytes, platelets, eosinophils, basophils, neutrophils, macrophages, and their stem cell precursors. The third class includes small peptide factors such as bombesin, vasopressin, oxytocin, endothelin, transferrin, angiotensin II, vasoactive intestinal peptide, and bradykinin which function as hormones to regulate cellular functions other than proliferation.

Growth and differentiation factors play critical roles in neoplastic transformation of cells in vitro and in tumor progression in vivo. Inappropriate expression of growth factors by tumor cells may contribute to vascularization and metastasis of tumors. During hematopoiesis, growth factor misregulation can result in anemias, leukemias, and lymphomas. Certain growth factors such as interferon are cytotoxic to tumor cells both in vivo and in vitro. Moreover, some growth factors and growth factor receptors are related both structurally and functionally to oncoproteins. In addition, growth factors affect transcriptional regulation of both proto-oncogenes and oncosuppressor genes. (Reviewed in Pimentel, E. (1994) Handbook of Growth Factors, CRC Press, Ann Arbor, Mich., pp. 1-9.)

The Slit protein, first identified in Drosophila, is critical in central nervous system midline formation and potentially in nervous tissue histogenesis and axonal pathfinding. Itoh et al. have identified mammalian homologues of the slit gene (human Slit-1, Slit-2, Slit-3 and rat Slit-1). The encoded proteins are putative secreted proteins containing EFG-like motifs and leucine-rich repeats, both are conserved protein-protein interaction domains. Slit-1, -2, and -3 mRNAs are expressed in the brain, spinal cord, and thyroid, respectively (Itoh, A. et al., (1998) Brain Res. Mol. Brain Res. 62:175-186). The Slit family of proteins are indicated to be functional ligands of glypican-1 in nervous tissue and suggests that their interactions may be critical in certain stages during central nervous system histogenesis (Liang, Y. et al., (1999) J. Biol. Chem. 274:17885-17892).

Neuropeptides and vasomediators (NPNM) comprise a large family of endogenous signaling molecules. Included in this family are neuropeptides and neuropeptide hormones such as bombesin, neuropeptide Y, neurotensin, neuromedin N, melanocortins, opioids, galanin, somatostatin, tachykinins, urotensin II and related peptides involved in smooth muscle stimulation, vasopressin, vasoactive intestinal peptide, and circulatory system-bome signaling molecules such as angiotensin, complement, calcitonin, endothelins, formyl-methionyl peptides, glucagon, cholecystokinin and gastrin. NPNVMs can transduce signals directly, modulate the activity or release of other neurotransmitters and hormones, and act as catalytic enzymes in cascades. The effects of NP/VMs range from extremely brief to long-lasting. (Reviewed in Martin, C. R. et al. (1985) Endocrine Physiology, Oxford University Press, New York, N.Y., pp. 57-62.)

NP/VMs are involved in numerous neurological and cardiovascular disorders. For example, neuropeptide Y is involved in hypertension, congestive heart failure, affective disorders, and appetite regulation. Somatostatin inhibits secretion of growth hormone and prolactin in the anterior pituitary, as well as inhibiting secretion in intestine, pancreatic acinar cells, and pancreatic beta-cells. A reduction in somatostatin levels has been reported in Alzheimer's disease and Parkinson's disease. Vasopressin acts in the kidney to increase water and sodium absorption, and in higher concentrations stimulates contraction of vascular smooth muscle, platelet activation, and glycogen breakdown in the liver. Vasopressin and its analogues are used clinically to treat diabetes insipidus. Endothelin and angiotensin are involved in hypertension, and drugs, such as captopril, which reduce plasma levels of angiotensin, are used to reduce blood pressure (Watson, S. and S. Arkinstall (1994) The G-protein Linked Receptor Facts Book, Academic Press, San Diego Calif., pp. 194; 252; 284; 55; 111).

Neuropeptides have also been shown to have roles in nociception (pain). Vasoactive intestinal peptide appears to play an important role in chronic neuropathic pain. Nociceptin, an endogenous ligand for for the opioid receptor-like 1 receptor, is thought to have a predominantly anti-nociceptive effect, and has been shown to have analgesic properties in different animal models of tonic or chronic pain (Dickinson, T. and Fleetwood-Walker, S. M. (1998) Trends Pharmacol. Sci. 19:346-348).

Other proteins that contain signal peptides include secreted proteins with enzymatic activity. Such activity includes, for example, oxidoreductase/dehydrogenase activity, transferase activity, hydrolase activity, lyase activity, isomerase activity, or ligase activity. For example, matrix metalloproteinases are secreted hydrolytic enzymes that degrade the extracellular matrix and thus play an important role in tumor metastasis, tissue morphogenesis, and arthritis (Reponen, P. et al. (1995) Dev. Dyn. 202:388-396; Firestein, G. S. (1992) Curr. Opin. Rheumatol. 4:348-354; Ray, J. M. and Stetler-Stevenson, W. G. (1994) Eur. Respir. J. 7:2062-2072; and Mignatti, P. and Rifkin, D. B. (1993) Physiol. Rev. 73:161-195). Additional examples are the acetyl-CoA synthetases which activate acetate for use in lipid synthesis or energy generation (Luong, A. et al. (2000) J. Biol. Chem. 275:26458-26466). The result of acetyl-CoA synthetase activity is the formation of acetyl-CoA from acetate and CoA. Acetyl-CoA sythetases share a region of sequence similarity identified as the AMP-binding domain signature. Acetyl-CoA synthetase has been shown to be associated with hypertension (H. Toh (1991) Protein Seq. Data Anal. 4:111-117 and Iwai, N. et al., (1994) Hypertension 23:375-380).

A number of isomerases catalyze steps in protein folding, phototransduction, and various anabolic and catabolic pathways. One class of isomerases is known as peptidyl-prolyl cis-trans isomerases (PPlases). PPIases catalyze the cis to trans isomerization of certain proline imidic bonds in proteins. Two families of PPIases are the FK506 binding proteins (FKBPs), and cyclophilins (CyPs). FKBPs bind the potent immunosuppressants FK506 and rapamycin, thereby inhibiting signaling pathways in T-cells. Specifically, the PPIase activity of FKBPs is inhibited by binding of FK506 or rapamycin. There are five members of the FKBP family which are named according to their calculated molecular masses (FKBP12, FKBP13, FKBP25, FKBP52, and FKBP65), and localized to different regions of the cell where they associate with different protein complexes (Coss, M. et al. (1995) J. Biol. Chem. 270:29336-29341; Schreiber, S. L. (1991) Science 251:283-287).

The peptidyl-prolyl isomerase activity of CyP may be part of the signaling pathway that leads to T-cell activation. CyP isomerase activity is associated with protein folding and protein trafficking, and may also be involved in assembly/disassembly of protein complexes and regulation of protein activity. For example, in Drosophila, the CyP NinaA is required for correct localization of rhodopsins, while a mammalian CyP (Cyp40) is part of the Hsp90/Hsc70 complex that binds steroid receptors. The mammalian CypA has been shown to bind the gag protein from human immunodeficiency virus 1 (HIV-1), an interaction that can be inhibited by cyclosporin. Since cyclosporin has potent anti-HlV-1 activity, CypA may play an essential function in HIV-1 replication. Finally, Cyp40 has been shown to bind and inactivate the transcription factor c-Myb, an effect that is reversed by cyclosporin. This effect implicates CyPs in the regulation of transcription, transformation, and differentiation (Bergsma, D. r. et al (1991) J. Biol. Chem. 266:23204-23214; Hunter, T. (1998) Cell 92: 141-143; and Leverson, J. D. and Ness, S. A. (1998) Mol. Cell. 1:203-211).

Molecular chaperones are a set of conserved protein families that recognize and selectively bind normative proteins under physiological and stress conditions. In this way these protein cofactors prevent irreversible aggregation reactions and misfolding. Many chaperones are also heat shock (stress) proteins. All major heat shock protein families (Hsp104, Hsp90, Hsp70, Hsp60/GroEL, and small Hsps) suppress irreversible unfolding reactions. They also function to maintain newly synthesized proteins in an unfolded conformation suitable for translocation across membranes. Bip (binding protein) is a homolog of the endoplasmic reticulum (ER) hsp70 protein. Bip (also known as Grp78 in mammalian cells or Kar2 in yeast), is involved in essentially all aspects of protein synthesis and secretion (Yu, M. et al. (2000) J. Biol. Chem. 275:24984-24992), and has been shown to transiently associate with newly synthesized secretory proteins including variant surface glycoprotein (VSG). Apg-1 and apg-2 belong to the hspl 10 family of heat shock proteins. The mouse apg-1 gene is structurally related to the human hsp70RY gene, and is inducible by a 32 to 39° C. heat shock. While apg-2 does seem to be an isoform of mouse homolog of hsp70RY, it does not appear to be heat-inducible (Nonoguchi, K. et al. (1999) Gene 237:21-28).

Gamma-carboxyglutamic acid (Gla) proteins rich in proline (PRGPs) are members of a family of vitamin Kdependent single-pass integral membrane proteins. These proteins are characterized by an extracellular amino terminal domain of approximately 45 amino acids rich in Gla. The intracellular carboxyl terminal region contains one or two copies of the sequence PPXY, a motif present in a variety of protiens involved in such diverse cellular functions as signal transduction, cell cycle progression, and protein turnover (Kulman, J. D. et al., (2001) Proc. Natl. Acad. Sci. U.S.A. 98:1370-1375). The process of post-translational modification of glutamic residues to form Gla is Vitamin Kdependent carboxylation. Proteins which contain Gla include plasma proteins involved in blood coagulation. These proteins are prothrombin, proteins C, S, and Z, and coagulation factors VII, IX, and X. Osteocalcin (bone-Gla protein, BGP) and matrix Gla-protein (MGP) also contain Gla (Friedman, P. A., and C. T. Przysiecki (1987) Int. J. Biochem. 19:1-3; C. Vermeer (1990) Biochem. J. 266:625-636).

The Drosophila sp. gene crossveinless 2 is characterized as having a putative signal or transmembrane sequence, and a partial Von Willebrand Factor D domain similar to those domains known to regulate the formation of intramolecular and intermolecular bonds and five cysteine-rich domains, known to bind BMP-like (bone morphogenetic proteins) ligands. These features suggest that crossveinless 2 may act extracelluarly or in the secretory pathway to directly potentiate ligand signaling and hence, involvement in the BMP-like signaling pathway known to play a role in vein specification (Conley, C. A. et al., (2000) Development 127:3947-3959). The dorsal-ventral patterning in both vertebrate and Drosophila embryos requires a conserved system of extracellular proteins to generate a positional informational gradient.

Antigen recognition molecules are key players in the sophisticated and complex immune systems which all vertebrates have developed to provide protection from viral, bacterial, fungal, and parasitic infections. A key feature of the immune system is its ability to distinguish foreign molecules, or antigens, from “self” molecules. This ability is mediated primarily by secreted and transmembrane proteins expressed by leukocytes (white blood cells) such as lymphocytes, granulocytes, and monocytes. Most of these proteins belong to the immunoglobulin (Ig) superfamily, members of which contain one or more repeats of a conserved structural domain. This Ig domain is comprised of antiparallel β sheets joined by a disulfide bond in an arrangement called the Ig fold. Members of the Ig superfamily include T-cell receptors, major histocompatibility (MHC) proteins, antibodies, and immune cell-specific surface markers such as the “cluster of differentiation” or CD antigens. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocvte Antigen Facts Book, Academic Press, San Diego, Calif., pp. 17-20.)

MHC proteins are cell surface markers that bind to and present foreign antigens to T cells. MHC molecules are classified as either class I or class II. Class I MHC molecules (MHC I) are expressed on the surface of almost all cells and are involved in the presentation of antigen to cytotoxic T cells. For example, a cell infected with virus will degrade intracellular viral proteins and express the protein fragments bound to MHC I molecules on the cell surface. The MHC Itantigen complex is recognized by cytotoxic T-cells which destroy the infected cell and the virus within. Class II MHC molecules are expressed primarily on specialized antigen-presenting cells of the immune system, such as B-cells and macrophages. These cells ingest foreign proteins from the extracellular fluid and express MHC I/antigen complex on the cell surface. This complex activates helper T-cells, which then secrete cytokines and other factors that stimulate the immune response. MHC molecules also play an important role in organ rejection following transplantation. Rejection occurs when the recipient's T-cells respond to foreign MHC molecules on the transplanted organ in the same way as to self MHC molecules bound to foreign antigen. (Reviewed in Alberts, B. et al. (1994) Molecular Biologv of the Cell, Garland Publishing, New York, N.Y., pp. 1229-1246.)

Antibodies, or immunoglobulins, are either expressed on the surface of B-cells or secreted by B-cells into the circulation. Antibodies bind and neutralize foreign antigens in the blood and other extracellular fluids. The prototypical antibody is a tetramer consisting of two identical heavy polypeptide chains (H-chains) and two identical light polypeptide chains (L-chains) interlinked by disulfide bonds. This arrangement confers the characteristic Y-shape to antibody molecules. Antibodies are classified based on their H-chain composition. The five antibody classes, IgA, IgD, IgE, IgG and IgM, are defined by the α, δ, ε, γ, and μ H-chain types. There are two types of L-chains, K and A, either of which may associate as a pair with any H-chain pair. IgG, the most common class of antibody found in the circulation, is tetrameric, while the other classes of antibodies are generally variants or multimers of this basic structure.

H-chains and L-chains each contain an N-terminal variable region and a C-terminal constant region. The constant region consists of about 110 amino acids in L-chains and about 330 or 440 amino acids in H-chains. The amino acid sequence of the constant region is nearly identical among H- or L-chains of a particular class. The variable region consists of about 110 amino acids in both H- and L-chains. However, the amino acid sequence of the variable region differs among H- or L-chains of a particular class. Within each H- or L-chain variable region are three hypervariable regions of extensive sequence diversity, each consisting of about 5 to 10 amino acids. In the antibody molecule, the H- and L-chain hypervariable regions come together to form the antigen recognition site. (Reviewed in Alberts, supra, pp. 1206-1213 and 1216-1217.)

Both H-chains and L-hains contain repeated Ig domains. For example, a typical H-chain contains four Ig domains, three of which occur within the constant region and one of which occurs within the variable region and contributes to the formation of the antigen recognition site. Likewise, a typical L-chain contains two Ig domains, one of which occurs within the constant region and one of which occurs within the variable region.

The immune system is capable of recognizing and responding to any foreign molecule that enters the body. Therefore, the immune system must be armed with a full repertoire of antibodies against all potential antigens. Such antibody diversity is generated by somatic rearrangement of gene segments encoding variable and constant regions. These gene segments are joined together by site-specific recombination which occurs between highly conserved DNA sequences that flank each gene segment. Because there are hundreds of different gene segments, millions of unique genes can be generated combinatorially. In addition, imprecise joining of these segments and an unusually high rate of somatic mutation within these segments further contribute to the generation of a diverse antibody population.

Expression Profiling

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Bone Remodeling and Osteoporosis

Bone remodeling occurs through teams of juxtaposed bone absorbing osteoclast and bone forming osteoblast and osteocyte cells. The development and proliferation of these cells from their progenitors is governed by networks of growth factors and cytokines produced in the bone microenvironment as well as by systematic hormones (Manolagas (1998) Aging 10:182-190; Teitelbaum et al. (1997) J Leukoc Biol 61:381-388). Coordinated balance between absorption and deposition is necessary to maintain bone integrity and requires intimate and complex interactions between osteoclasts and osteoblasts. Under normal states of bone homeostasis, the remodeling activities in bone serve to remove bone mass where the mechanical demands of the skeleton are low and form bone at the those sites where mechanical loads are repeatedly transmitted.

Bone is a composite material composed of an organic and an inorganic phase. By weight, approximately 70% of the tissue is mineral or inorganic matter (mainly calcium phosphate); water comprises 5 to 8%; and, the organic or extracellular matrix makes up the remainder. Approximately 95% of the mineral phase is composed of a specific crystalline hydroxyapatite that is impregnated with impurities which make up the remaining 5% of the inorganic phase. Ninety-eight percent of the organic phase is composed of type I collagen and a variety of non-collagenous proteins; cells make up the remaining 2% of this phase (Einhom (1996) The bone organ system: form and function. In: Marcus et al. eds., Osteoporosis, Academic Press, New York N.Y.). The process of matrix deposition by osteoblasts and osteocytes, subsequent mineralization and the coupling with bone resorbing activity of osteoclasts is governed by a complex interplay of systemic hormones, peptides and downstream signaling pathway proteins, local transcription factors, cytokines, growth factors and matrix remodeling genes.

Parathyroid hormone (PTH) and its signaling system are the principal regulators of bone remodeling in the adult skeleton (Masiukiewicz and Insogna (1998) Aging 10:232-239; Mierke and Pellegrini (1999) Curr Pharm Des 5:21-36). They have a vital role in the homeostasis of calcium within the blood stream and the acute in vivo effect of PTH is to increase bone resorption, although sustained increases in its circulating levels accelerate both formation and resorption. The PTH signaling pathway may also be involved in the regulation of chondrogenesis during bone formation (Vortkamp et al. (1996) Science 273:613-622; Lanske et al. (1999) J Clin Invest 104:399-407).

Several other hormones and local factors are vital to bone health. In a complex pattern of inhibition and stimulation not yet fully understood, growth hormone, insulin-like growth factor-1, the sex steroids, thyroid hormone, calciotrophic hormones such as PTH and prostaglandin E2, various cytokines, such as interleukin-1 beta, interleukin-6, and tumor necrosis factor-alpha, and 1,25-dihydroxyvitamin D (calcitriol) all act coordinately in the bone remodeling process. Estrogen is involved in inhibition of osteoclast activity (Jilka et al. (1992) Science 257:88-91; Poli et al. (1994) EMBO J. 13:1189-1196; Srivastava et al. (1998) J Clin Invest 102:1850-1859). Estrogen may prevent bone loss by blocking the production of cytokines in bone or bone marrow (Kimble et al. (1995) Endocrinology 136:3054-3061). Various cytokines, such as interleukin-1 beta, interleukin-6, and tumor necrosis factor-alpha, influence bone remodeling (de Vernejoul (1996) Eur J Clin Chem Clin Biochem 34:729-734).

Throughout life, old bone is removed (resorption) and new bone is added to the skeleton (formation). During childhood and teenage years, new bone is added faster than old bone is removed. As a result, bones become larger, heavier and denser. Bone formation continues at a pace faster than resorption until peak bone mass is reached during the mid-20s. After age 30, bone resorption slowly begins to exceed bone formation, most rapidly in the first few years after menopause but persistantly until death. Osteoporosis develops when bone resorption occurs too quickly or if replacement occurs too slowly. Two major classes of osteoporosis are primary and secondary osteoporosis. Type I osteoporosis occurs in a subset of postmenopausal women who are between 50 and 70 years of age and is associated with fractures of vertebral bodies and the forearm. Type II osteoporosis occurs in women and men over the age of 70 and is associated with fractures of the femoral neck and proximal humerus and tibia. In some instances, osteoporosis is a manifestation of another disease (Fauci et al. (1998) Harrison's Principles of Internal Medicine, McGraw Hill Companies, New York N.Y., pp 2249). Current therapies are designed to interfere with these growth regulatory systems to encourage the growth and function of osteoblasts and inhibit the growth and activity of osteoclasts.

Extracellular Matrix in Cancer

Normal tissue homeostasis is maintained by dynamic interactions between epithelial cells and their microenvironment. As tissue becomes cancerous, reciprocal interactions occur between neoplastic cells, adjacent normal cells such as stroma and endothelium, and their microenvironments. The latter is defined as insoluble extracellular matrix (ECM); stroma consisting of fibroblasts, adipose cells, vasculature, resident immune cells, and the conventional milieu of cytokines and growth factors. Epithelial parenchyma are physically separated from stroma by a basement membrane: a highly organized, special ECM, whose composition is different from stromal ECM and to which epithelial cells attach. Cell biology has fumly established in model systems that the complex interactions between epithelial cells and their microenvironment are critical for maintaining normal, balanced homeostasis. Disruption of this balance can induce aberrant cell proliferation, adhesion, function, and migration that might promote malignant and metastatic behavior. In vitro use of ECM provides cells with conditions that more closely approximate their in vivo physiologic environments. Matrigel matrix is a reconstituted basement membrane isolated from the EHS mouse sarcoma, a tumor rich in ECM proteins. Matrigel matrix is composed of laminin, collagen IV, entactin, and heparin sulfate proteoglycan. It also contains growth factors, matrix metalloproteinases, and other components. Cells normally in contact with a basement membrane in vivo often are well differentiated when cultured on Matrigel basement membrane matrix in vitro. Understanding the contribution of the microenvironment to the development and metastasis of cancer, and how to manipulate the interactions between cancer cells and the microenvironment, might lead to novel therapeutic targets.

The discovery of new secreted proteins, and the polynucleotides encoding them, satisfies a need in the art by providing new compositions which are useful in the diagnosis, prevention, and treatment of cell proliferative, autoimmune/inflammatory, cardiovascular, neurological, and developmental disorders, and in the assessment of the effects of exogenous compounds on the expression of nucleic acid and amino acid sequences of secreted proteins.

### SUMMARY OF THE INVENTION

The invention features purified polypeptides, secreted proteins, referred to collectively as “SECP” and individually as “SECP-1,” “SECP-2,” “SECP-3,” “SECP4,” “SECP-5,” “SECP-6,” “SECP-7,” “SECP-8,” “SECP-9,” “SECP-10,” “SECP-1,” “SECP-12,” “SECP-13,” “SECP-14,” “SECP-15,” “SECP-16,” “SECP-17,” “SECP-18,” “SECP-19,” “SECP-20,” “SECP-21,” “SECP-22,” “SECP-23,” “SECP-24,” “SECP-25,” “SECP-26,” “SECP-27,” “SECP-28,” “SECP-29,” “SECP-30,” “SECP-31,” and “SECP-32.” In one aspect, the invention provides an isolated polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. In one alternative, the invention provides an isolated polypeptide comprising the amino acid sequence of SEQ ID NO:1-32.

The invention further provides an isolated polynucleotide encoding a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. In one alternative, the polynucleotide encodes a polypeptide selected from the group consisting of SEQ ID NO:1-32. In another alternative, the polynucleotide is selected from the group consisting of SEQ ID NO:33-64.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32.

The invention further provides an isolated polynucleotide selected from the group consisting of a) a polynucleotide comprising a polynucleotide sequence selected from the group consisting of SEQ ID NO:3364, b) a polynucleotide comprising a naturally occurring polynucleotide sequence at least 90% identical to a polynucleotide sequence selected from the group consisting of SEQ ID NO:33-64, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:33-64, b) a polynucleotide comprising a naturally occurring polynucleotide sequence at least 90% identical to a polynucleotide sequence selected from the group consisting of SEQ ID NO:33-64, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:33-64, b) a polynucleotide comprising a naturally occurring polynucleotide sequence at least 90% identical to a polynucleotide sequence selected from the group consisting of SEQ ID NO:33-64, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polyriucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID) NO: 1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32, and a pharmaceutically acceptable excipient. In one embodiment, the composition comprises an amino acid sequence selected from the group consisting of SEQ ID NO:1-32. The invention additionally provides a method of treating a disease or condition associated with decreased expression of functional SECP, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional SECP, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional SECP, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-32, b) a polypeptide comprising a naturally occurring amino acid sequence at least 90% identical to an amino acid sequence selected from the group consisting of SEQ ID NO:1-32, c) a biologically active fragment of a polypeptide having an amino acid sequence selected from the group consisting of SEQ ID NO:1-32, and d) an immunogenic fragment of a polypeptide having an amino acid sequence selected from the group consisting of SEQ ID NO:1-32. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:33-64, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:33-64, ii) a polynucleotide comprising a naturally occurring polynucleotide sequence at least 90% identical to a polynucleotide sequence selected from the group consisting of SEQ ID NO:33-64, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:33-64, ii) a polynucleotide comprising a naturally occurring polynucleotide sequence at least 90% identical to a polynucleotide sequence selected from the group consisting of SEQ ID NO:33-64, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 surnmarizes the nomenclature for the full length polynucleotide and polypeptide sequences of the present invention.

Table 2 shows the GenBank identification number and annotation of the nearest GenBank homolog for polypeptides of the invention. The probability scores for the matches between each polypeptide and its homolog(s) are also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“SECP” refers to the amino acid sequences of substantially purified SECP obtained from any species, particularly a manmmalian species, including bovine, ovine, porcine, murine, equine, and human, and from any source, whether natural, synthetic, semi-synthetic, or recombinant.

The term “agonist” refers to a molecule which intensifies or mimics the biological activity of SECP. Agonists may include proteins, nucleic acids, carbohydrates, small molecules, or any other compound or composition which modulates the activity of SECP either by directly interacting with SECP or by acting on components of the biological pathway in which SECP participates.

An “allelic variant” is an alternative form of the gene encoding SECP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding SECP include those sequences with deletions, insertions, or substitutions of different nucleotides, resulting in a polypeptide the same as SECP or a polypeptide with at least one functional characteristic of SECP. Included within this definition are polymorphisms which may or may not be readily detectable using a particular oligonucleotide probe of the polynucleotide encoding SECP, and improper or unexpected hybridization to allelic variants, with a locus other than the normal chromosomal locus for the polynucleotide sequence encoding SECP. The encoded protein may also be “altered,” and may contain deletions, insertions, or substitutions of amino acid residues which produce a silent change and result in a functionally equivalent SECP. Deliberate amino acid substitutions may be made on the basis of similarity in polarity, charge, solubility, hydrophobicity, hydrophilicity, and/or the amphipathic nature of the residues, as long as the biological or immunological activity of SECP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of SECP. Antagonists may include proteins such as antibodies, nucleic acids, carbohydrates, small molecules, or any other compound or composition which modulates the activity of SECP either by directly interacting with SECP or by acting on components of the biological pathway in which SECP participates.

The term “antibody” refers to intact immunoglobulin molecules as well as to fragments thereof, such as Fab, F(ab′)₂, and Fv fragments, which are capable of binding an epitopic determinant. Antibodies that bind SECP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) Y. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic SECP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding SECP or fragments of SECP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of SECP or the polynucleotide encoding SECP which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:33-64 comprises a region of unique polynucleotide sequence that specifically identifies SEQ ID NO:33-64, for example, as distinct from any other sequence in the genome from which the fragment was obtained. A fragment of SEQ ID NO:33-64 is useful, for example, in hybridization and amplification technologies and in analogous methods that distinguish SEQ ID NO:33-64 from related polynucleotide sequences. The precise length of a fragment of SEQ ID NO:33-64 and the region of SEQ ID NO:33-64 to which the fragment corresponds are routinely determinable by one of ordinary skill in the art based on the intended purpose for the fragment.

A fragment of SEQ ID NO:1-32 is encoded by a fragment of SEQ ID NO:3364. A fragment of SEQ ID NO:1-32 comprises a region of unique amino acid sequence that specifically identifies SEQ ID NO:1-32. For example, a fragment of SEQ ID NO:1-32 is useful as an immunogenic peptide for the development of antibodies that specifically recognize SEQ ID NO:1-32. The precise length of a fragment of SEQ ID NO:1-32 and the region of SEQ ID NO:1-32 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (April-21-2000) set at default parameters. Such default parameters may be, for example:

Matrix: BLOSUM62

Reward for match: 1

Penalty for mismatch: −2

Open Gap: 5 and Extension Gap: 2 penalties

Gap×drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise aligrnents of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (April-21-2000) with blastp set at default parameters. Such default parameters mray be, for example:

Matrix: BLOSUM62

Open Gap: 11 and Extension Gap: 1 penalties

Gap×drop-off. 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6× SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2× SSC and about 0.1% SDS, for 1 hour. Alternatively, temperatures of about 65° C., 60° C., 55° C., or 42° C. may be used. SSC concentration may be varied from about 0.1 to 2× SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of SECP which is capable of eliciting an immune response when introduced into a living organism, for example, a mammal. The term “immunogenic fragment” also includes any polypeptide or oligopeptide fragment of SECP which is useful in any of the antibody production methods disclosed herein or known in the art.

The term “ricroarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of SECP. For example, modulation may cause an increase or a decrease in protein activity, binding characteristics, or any other biological, functional, or immunological properties of SECP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an SECP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of SECP.

“Probe” refers to nucleic acid sequences encoding SECP, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratorv Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the manmmal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing SECP, nucleic acids encoding SECP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In one alternative, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human secreted proteins (SECP), the polynucleotides encoding SECP, and the use of these compositions for the diagnosis, treatment, or prevention of cell proliferative, autoimmune/inflarnrnatory, cardiovascular, neurological, and developmental disorders.

Table 1 sumnaarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s).

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 sumnmarize the properties of polypeptides of the invention, and these properties establish that the claimed polypeptides are secreted proteins.

For example, SEQ ID NO:1 is 934 residues in length, and is 95% identical, from residue L146 to residue D934, to human Ig-like membrane protein (GenBank ID g3766136) as determined by the Basic Local Alignment Search Tool (BLAST). (See Table 2.) The BLAST probability score is 0.0, which indicates the probability of obtaining the observed polypeptide sequence alignment by chance. SEQ ID NO:1 also contains immunoglobulin domains as determined by searching for statistically significant matches in the hidden Markov model (HMM)-based PFAM database of conserved protein family domains.

As another example, SEQ ID NO:10 is 33% identical, from residue Y296 to residue G398, 35% identical, from residue C418 to residue E481, 31% identical, from residue E196 to L271, and 29% identical, from residue G534 to residue P594 to Trypanosoma cruzi GP634 protein, a surface glycoprotein (GenBank ID g8101109) as determined by the Basic Local Alignment Search Tool (BLAST). (See Table 2.) The BLAST probability score is 1.9e-19, which indicates the probability of obtaining the observed polypeptide sequence alignment by chance. SEQ ID NO:10 also contains leishmanolysin metalloprotease domains as determined by searching for statistically significant matches in the hidden Markov model (HMM)-based PFAM database of conserved protein family domains. (See Table 3.) Data from BLIMPS, and additional BLAST analyses against the PRODOM and DOMO databases provide further corroborative evidence that SEQ ID NO:10 is a metalloprotease.

As another example, SEQ ID NO:12 is 98% identical, from residue MI to residue P419, to human pregnancy-specific beta 1-glycoprotein 7 precursor (GenBank ID g609314) as determined by the Basic Local Alignment Search Tool (BLAST). (See Table 2.) The BLAST probability score is 6.0e-229, which indicates the probability of obtaining the observed polypeptide sequence alignment by chance.

As another example, SEQ ID NO:24 contains an immunoglobulin domain, a fibronectin type III domain, and leucine rich repeat domains as determined by searching for statistically significant matches in the hidden Markov model (HMM)-based PFAM database of conserved protein family domains. (See Table 2.) Note that “immunoglobulin domains” and “fibronectin domains” are distinguishing motifs which are characteristic of matrix proteins, one type of secreted protein.

As another example, SEQ ID NO:25 is 97% identical, from residue MI to residue D841, to human apg-2 protein (GenBank ID g4579909) as determined by the Basic Local Alignment Search Tool (BLAST). (See Table 2.) The BLAST probability score is 0.0, which indicates the probability of obtaining the observed polypeptide sequence alignment by chance. SEQ ID NO:25 also contains an Hsp70 protein domain as determined by searching for statistically significant matches in the hidden Markov model (HMM)-based PFAM database of conserved protein family domains. (See Table 3.) Data from BLIMPS, BLAST, and MOTIFS analyses provide further corroborative evidence that SEQ ID NO:25 is a secreted protein molecule. SEQ ID NO:2-9, SEQ ID NO:11, SEQ ID NO:13-23, and SEQ ID NO₂₆-32 were analyzed and annotated in a similar manner. The algorithms and parameters for the analysis of SEQ ID NO:1-32 are described in Table 7.

As shown in Table 4, the full length polynucleotide sequences of the present invention were assembled using cDNA sequences or coding (exon) sequences derived from genomnic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide sequences of the invention, and of fragments of the polynucleotide sequences which are useful, for example, in hybridization or amplification technologies that identify SEQ ID NO:33-64 or that distinguish between SEQ ID NO:33-64 and related polynucleotide sequences.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotide sequences. In addition, the polynucleotide fragments described in column 2 may identify sequences derived from the ENSEMBL (The Sanger Centre, Cambridge, UK) database (ie., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (ie., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_N_{2—}YYYYY_N_{3—N}₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., GBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example I and Example V).

| Prefix | Type of analysis and/or examples of programs |
|---|---|
| GNN, GFG, | Exon prediction from genomic sequences using, |
| ENST | for example, GENSCAN (Stanford University, |
| | CA, USA) or FGENES (Computer Genomics Group, |
| | The Sanger Centre, Cambridge, UK). |
| GBI | Hand-edited analysis of genomic sequences. |
| FL | Stitched or stretched genomic sequences |
| | (see Example V). |
| INCY | Full length transcript and exon prediction from |
| | mapping of EST sequences to the genome. |
| | Genomic location and EST composition |
| | data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP(SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses SECP variants. A preferred SECP variant is one which has at least about 80%, or alternatively at least about 90%, or even at least about 95% amino acid sequence identity to the SECP amino acid sequence, and which contains at least one functional or structural characteristic of SECP.

The invention also encompasses polynucleotides which encode SECP. In a particular embodiment, the invention encompasses a polynucleotide sequence comprising a sequence selected from the group consisting of SEQ ID NO:33-64, which encodes SECP. The polynucleotide sequences of SEQ ID NO:33-64, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding SECP. In particular, such a variant polynucleotide sequence will have at least about 70%, or alternatively at least about 85%, or even at least about 95% polynucleotide sequence identity to the polynucleotide sequence encoding SECP. A particular aspect of the invention encompasses a variant of a polynucleotide sequence comprising a sequence selected from the group consisting of SEQ ID NO:33-64 which has at least about 70%, or alternatively at least about 85%, or even at least about 95% polynucleotide sequence identity to a nucleic acid sequence selected from the group consisting of SEQ ID NO:33-64. Any one of the polynucleotide variants described above can encode an amino acid sequence which contains at least one functional or structural characteristic of SECP.

In addition, or in the alternative, a polynucleotide variant of the invention is a splice variant of a polynucleotide sequence encoding SECP. A splice variant may have portions which have significant sequence identity to the polynucleotide sequence encoding SECP, but will generally have a greater or lesser number of polynucleotides due to additions or deletions of blocks of sequence arising from alternate splicing of exons during mNA processing. A splice variant rnay have less than about 70%, or alternatively less than about 60%, or alternatively less than about 50% polynucleotide sequence identity to the polynucleotide sequence encoding SECP over its entire length; however, portions of the splice variant will have at least about 70%, or alternatively at least about 85%, or alternatively at least about 95%, or alternatively 100% polynucleotide sequence identity to portions of the polynucleotide sequence encoding SECP. For example, a polynucleotide comprising a sequence of SEQ ID NO:63 is a splice variant of a polynucleotide comprising a sequence of SEQ ID NO:48,a polynucleotide comprising a sequence of SEQ ID NO:64 is a splice variant of a polynucleotide comprising a sequence of SEQ ID NO:53, and a polynucleotide comprising a sequence of SEQ ID NO:62 is a splice variant of a polynucleotide comprising a sequence of SEQ ID NO:58. Any one of the splice variants described above can encode an amino acid sequence which contains at least one functional or structural characteristic of SECP.

It will be appreciated by those skilled in the art that as a result of the degeneracy of the genetic code, a multitude of polynucleotide sequences encoding SECP, some bearing minimal similarity to the polynucleotide sequences of any known and naturally occurring gene, may be produced. Thus, the invention contemplates each and every possible variation of polynucleotide sequence that could be made by selecting combinations based on-possible codon choices. These combinations are made in accordance with the standard triplet genetic code as applied to the polynucleotide sequence of naturally occurring SECP, and all such variations are to be considered as being specifically disclosed.

Although nucleotide sequences which encode SECP and its variants are generally capable of hybridizing to the nucleotide sequence of the naturally occurring SECP under appropriately selected conditions of stringency, it may be advantageous to produce nucleotide sequences encoding SECP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding SECP and its derivatives without altering the encoded amino acid sequences include the production of RNA transcripts having more desirable properties, such as a greater half-life, than transcripts produced from the naturally occurring sequence.

The invention also encompasses production of DNA sequences which encode SECP and SECP derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding SECP or any fragment thereof.

Also encompassed by the invention are polynucleotide sequences that are capable of hybridizing to the claimed polynucleotide sequences, and, in particular, to those shown in SEQ ID NO: 33-64 and fragments thereof under various conditions of stringency. (See, e.g., Wahl, G. M. and S. L. Berger (1987) Methods Enzymol. 152:399407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase 1, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding SECP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode SECP may be cloned in recombinant DNA molecules that direct expression of SECP, or fragments or functional equivalents thereof, in appropriate host cells. Due to the inherent degeneracy of the genetic code, other DNA sequences which encode substantially the same or a functionally equivalent amino acid sequence may be produced and used to express SECP.

The nucleotide sequences of the present invention can be engineered using methods generally known in the art in order to alter SECP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of SECP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding SECP may be synthesized, in whole or in part, using chemical methods well known in the art. (See, e.g., Caruthers, M. H. et al. (1980) Nucleic Acids Symp. Ser. 7:215-223; and Hom, T. et al. (1980) Nucleic Acids Symp. Ser. 7:225-232.) Alternatively, SECP itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of SECP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active SECP, the nucleotide sequences encoding SECP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding SECP. Such elements may vary in their strength and specificity. Specific initiation signals may also be used to achieve more efficient translation of sequences encoding SECP. Such signals include the ATG initiation codon and adjacent sequences, e.g. the Kozak sequence. In cases where sequences encoding SECP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding SECP and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding SECP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or aninal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I.M. and N. Somia (1997) Nature 389:239-242.) The invention is not limited by the host cell employed.

In bacterial systems, a number of cloning and expression vectors may be selected depending upon the use intended for polynucleotide sequences encoding SECP. For example, routine cloning, subcloning, and propagation of polynucleotide sequences encoding SECP can be achieved using a multifunctional E. coli vector such as PBLUESCRIPT (Stratagene, La Jolla Calif.) or PSPORT1 plasmid (Invitrogen). Ligation of sequences encoding SECP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of SECP are needed, e.g. for the production of antibodies, vectors which direct high level expression of SECP may be used. For example, vectors containing the strong, inducible SP6 or T7 bacteriophage promoter may be used.

Yeast expression systems may be used for production of SECP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, surra; Bitter, G. A. et al. (1987) Methods Enzymol. 153:516-544; and Scorer, C. A. et al. (1994) Bio/Technology 12:181-184.)

Plant systems may also be used for expression of SECP. Transcription of sequences encoding SECP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mamnalian cells, a number of viral-based expression systems may be utilized. In cases where an adenovirus is used as an expression vector, sequences encoding SECP may be ligated into an adenovirus transcription/translation complex consisting of the late promoter and tripartite leader sequence. Insertion in a non-essential E1 or E3 region of the viral genome may be used to obtain infective virus which expresses SECP in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in marnmalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of SECP in cell lines is preferred. For example, sequences encoding SECP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G418; and als and pat confer resistance to chlorsulfuron and phosphinotricin acetyltransferase, respectively. (See, e.g., Wigler, M. et al. (1980) Proc. Natl. Acad. Sci. USA 77:3567-3570; ColbereGarapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), B glucuronidase and its substrate 6-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding SECP is inserted within a marker gene sequence, transformed cells containing-sequences encoding SECP can be identified by the absence of marker gene function. Alternatively, a marker gene can be placed in tandem with a sequence encoding SECP under the control of a single promoter. Expression of the marker gene in response to induction or selection usually indicates expression of the tandem gene as well.

In general, host cells that contain the nucleic acid sequence encoding SECP and that express SECP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of SECP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on SECP is preferred, but a competitive binding assay may be employed. These and other assays are well known in the art. (See, e.g., Hampton, R. et al. (1990) Serolopical Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding SECP include oligolabeling, nick translation, end-labeling, or PCR amplification using a labeled nucleotide. Alternatively, the sequences encoding SECP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding SECP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode SECP may be designed to contain signal sequences which direct secretion of SECP through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

In another embodiment of the invention, natural, modified, or recombinant nucleic acid sequences encoding SECP may be ligated to a heterologous sequence resulting in translation of a fusion protein in any of the aforementioned host systems. For example, a chimeric SECP protein containing a heterologous moiety that can be recognized by a cornmercially available antibody may facilitate the screening of peptide libraries for inhibitors of SECP activity. Heterologous protein and peptide moieties may also facilitate purification of fusion proteins using conmmercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the SECP encoding sequence and the heterologous protein sequence, so that SECP may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled SECP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

SECP of the present invention or fragments thereof may be used to screen for compounds that specifically bind to SECP. At least one and up to a plurality of test compounds may be screened for specific binding to SECP. Examples of test compounds include antibodies, oligonucleotides, proteins (e.g., ligands or receptors), or small molecules. In one embodiment, the compound thus identified is closely related to the natural ligand of SECP, e.g., a ligand or fragment thereof, a natural substrate, a structural or functional mimetic, or a natural binding partner. (See, e.g., Coligan, J. E. et al. (1991) Current Protocols in Immunology 1(2):Chapter 5.) In another embodiment, the compound thus identified is a natural ligand of a receptor SECP. (See, e.g., Howard, A. D. et al. (2001) Trends Pharmacol. Sci. 22:132-140; Wise, A. et al. (2002) Drug Discovery Today 7:235-246.)

In other embodiments, the compound can be closely related to the natural receptor to which SECP binds, at least a fragment of the receptor, or a fragment of the receptor including all or a portion of the ligand binding site or binding pocket. For example, the compound may be a receptor for SECP which is capable of propagating a signal, or a decoy receptor for SECP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Immunex Corp., Seattle Wash.), which is efficacious for treating rheumatoid arthritis in humans. Etanercept is an engineered p75 tumor necrosis factor (TNF) receptor dimer linked to the Fc portion of human IgG, (Taylor, P. C. et al. (2001) Curr. Opin. Immunol. 13:611-616).

In one embodiment, screening for compounds which specifically bind to, stimulate, or inhibit SECP involves producing appropriate cells which express SECP, either as a secreted protein or on the cell membrane. Preferred cells include cells from mammals, yeast, Drosophila, or E. coli. Cells expressing SECP or cell membrane fractions which contain SECP are then contacted with a test compound and binding, stimulation, or inhibition of activity of either SECP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with SECP, either in solution or affixed to a solid support, and detecting the binding of SECP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands. (See, e.g., Matthews, D. J. and J. A. Wells. (1994) Chem. Biol. 1:25-30.) In another related embodiment, one or more amino acid substitutions can be introduced into a polypeptide compound (such as a ligand) to improve or alter its ability to bind to its natural receptors. (See, e.g., Cunningham, B. C. and J. A. Wells (1991) Proc. Natl. Acad. Sci. USA 88:3407-3411; Lowman, H. B. et al. (1991) J. Biol. Chem. 266:10982-10988.)

SECP of the present invention or fragments thereof may be used to screen for compounds that modulate the activity of SECP. Such compounds may include agonists, antagonists, or partial or inverse agonists. In one embodiment, an assay is performed under conditions permissive for SECP activity, wherein SECP is combined with at least one test compound, and the activity of SECP in the presence of a test compound is compared with the activity of SECP in the absence of the test compound. A change in the activity of SECP in the presence of the test compound is indicative of a compound that modulates the activity of SECP. Alternatively, a test compound is combined with an in vitro or cell-free system comprising SECP under conditions suitable for SECP activity, and the assay is performed. In either of these assays, a test compound which modulates the activity of SECP may do so indirectly and need not come in direct contact with the test compound. At least one and up to a plurality of test compounds may be screened.

In another embodiment, polynucleotides encoding SECP or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BIJ6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding SECP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding SECP can also be used to create “knockin” humanized animals (pigs) or transgenic animals (mice or rats) to model human disease. With knockin technology, a region of a polynucleotide encoding SECP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress SECP, e.g., by secreting SECP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of SECP and secreted proteins. In addition, expression of SECP is closely associated with: adrenal, brain, fetal thymus, breast, thyroid, ovary, breast tumor, dorsal root ganglion, heart, nasal polyp, pancreas, ileum, and pineal gland tissues, neurological, reproductive, and fetal tissues, and with tissues associated with Alzheimer's disease. Further examples of tissues expressing SECP can be found in Table 6. Therefore, SECP appears to play a role in cell proliferative, autoimmune/inflammatory, cardiovascular, neurological, and developmental disorders. In the treatment of disorders associated with increased SECP expression or activity, it is desirable to decrease the expression or activity of SECP. In the treatment of disorders associated with decreased SECP expression or activity, it is desirable to increase the expression or activity of SECP.

Therefore, in one embodiment, SECP or a fragment or derivative thereof may be administered to a subject to treat or prevent a disorder associated with decreased expression or activity of SECP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scieroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidisrn, hydrocephalus, seizure disorders such as Syndenham's chorea and cerebral palsy, spina bifida, anencephaly, craniorachischisis, congenital glaucoma, cataract, and sensorineural hearing loss.

In another embodiment, a vector capable of expressing SECP or a fragment or derivative thereof may be administered to a subject to treat or prevent a disorder associated with decreased expression or activity of SECP including, but not limited to, those described above.

In a further embodiment, a composition comprising a substantially purified SECP in conjunction with a suitable pharmaceutical carrier may be administered to a subject to treat or prevent a disorder associated with decreased expression or activity of SECP including, but not limited to, those provided above.

In still another embodiment, an agonist which modulates the activity of SECP may be administered to a subject to treat or prevent a disorder associated with decreased expression or activity of SECP including, but not limited to, those listed above.

In a further embodiment, an antagonist of SECP may be administered to a subject to treat or prevent a disorder associated with increased expression or activity of SECP. Examples of such disorders include, but are not limited to, those cell proliferative, autoimmune/inflammatory, cardiovascular, neurological, and developmental disorders described above. In one aspect, an antibody which specifically binds SECP may be used directly as an antagonist or indirectly as a targeting or delivery mechanism for bringing a pharmaceutical agent to cells or tissues which express SECP.

In an additional embodiment, a vector expressing the complement of the polynucleotide encoding SECP may be administered to a subject to treat or prevent a disorder associated with increased expression or activity of SECP including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of SECP may be produced using methods which are generally known in the art. In particular, purified SECP may be used to produce antibodies or to screen libraries of pharmaceutical agents to identify those which specifically bind SECP. Antibodies to SECP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, hurmans, and others may be immunized by injection with SECP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corvnebacterium parvum are especially preferable.

It is preferred that the oligopeptides, peptides, or fragments used to induce antibodies to SECP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of SECP amino acids may be fused with those of another protein, such as KLH, and antibodies to the chimeric molecule may be produced.

Monoclonal antibodies to SECP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:68516855; Neuberger, M. S. et al. (1984) Nature 312:604608; and Takeda, S. et al. (1985) Nature 314:452-454.) Alternatively, techniques described for the production of single chain antibodies may be adapted, using methods known in the art, to produce SECP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for SECP may also be generated. For example, such fragments include, but are not limited to, F(ab)₂ fragments produced by pepsin digestion of the antibody molecule and Fab fragments generated by reducing the disulfide bridges of the F(ab)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between SECP and its specific antibody. A two-site, monoclonal-based immunoassay utilizing monoclonal antibodies reactive to two non-interfering SECP epitopes is generally used, but a competitive binding assay may also be employed (Pound, supra).

Various methods such as Scatchard analysis in conjunction with radioimmunoassay techniques may be used to assess the affinity of antibodies for SECP. Affinity is expressed as an association constant, Kₐ, which is defined as the molar concentration of SECP-antibody complex divided by the molar concentrations of free antigen and free antibody under equilibrium conditions. The Kₐ determined for a preparation of polyclonal antibodies, which are heterogeneous in their affinities for multiple SECP epitopes, represents the average affinity, or avidity, of the antibodies for SECP. The Kₐ determined for a preparation of monoclonal antibodies, which are monospecific for a particular SECP epitope, represents a true measure of affinity. High-affinity antibody preparations with Kₐ ranging from about 10⁹ to 10¹² L/mole are preferred for use in immunoassays in which the SECP-antibody complex must withstand rigorous manipulations. Low-affinity antibody preparations with Kₐ ranging from about 10⁶ to 10⁷ L/mole are preferred for use in immunopurification and similar procedures which ultimately require dissociation of SECP, preferably in active form, from the antibody (Catty, D. (1988) Antibodies, Volume I: A Practical Approach, IRL Press, Washington D. C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of SECP-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding SECP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding SECP. Such technology is well known in the art, and antisense oligonucleotides or larger fragments can be designed from various locations along the coding or control regions of sequences encoding SECP. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469475; and Scanlon, K. J. et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding SECP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIfI or Factor IX deficiencies (Crystal, R. G. (1995) Science 270:404410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in SECP expression or regulation causes disease, the expression of SECP from an appropriate population of transduced cells may alleviate the clinical manifestations caused by the genetic deficiency.

In a further embodiment of the invention, diseases or disorders caused by deficiencies in SECP are treated by constructing mammalian expression vectors encoding SECP and introducing these vectors by mechanical means into SECP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Recipon (1998) Curr. Opin. Biotechnol. 9:445-450).

Expression vectors that may be effective for the expression of SECP include, but are not limited to, the PcNA 3.1, EPITAG, PRCCMV2, PREP, PVAX, PCR2-TOPOTA vectors (Invitrogen, Carlsbad Calif.), PCMV-SCRIFT, PCMV-TAG, PEGSH(PERV (Stratagene, La Jolla Calif.), and PTET-OFF, PTET-ON, PTRE2, PTRE2-LUC, PTK-HYG (Clontech, Palo Alto Calif.). SECP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451456), comnercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding SECP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to SECP expression are treated by constructing a retrovirus vector consisting of (i) the polynucleotide encoding SECP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding SECP to cells which have one or more genetic abnormalities with respect to the expression of SECP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding SECP to target cells which have one or more genetic abnormalities with respect to the expression of SECP. The use of herpes simplex virus (HSV)-based vectors may be especially valuable for introducing SECP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding SECP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for SECP into the alphavirus genome in place of the capsid-coding region results in the production of a large number of SECP-coding RNAs and the synthesis of high levels of SECP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of SECP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding SECP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding SECP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding SECP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased SECP expression or activity, a compound which specifically inhibits expression of the polynucleotide encoding SECP may be therapeutically useful, and in the treatment of disorders associated with decreased SECP expression or activity, a compound which specifically promotes expression of the polynucleotide encoding SECP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding SECP is exposed to at least one test compound thus obtained. The sample may comprise, for example, an intact or permeabilized cell, or an in vitro cell-free or reconstituted biochemical system. Alterations in the expression of a polynucleotide encoding SECP are assayed by any method commonly known in the art. Typically, the expression of a specific nucleotide is detected by hybridization with a probe having a nucleotide sequence complementary to the sequence of the polynucleotide encoding SECP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are conmmonly known and are thoroughly discussed in the latest edition of Remington's Pharmaceutical Sciences (Maack Publishing, Easton Pa.). Such compositions may consist of SECP, antibodies to SECP, and mimetics, agonists, antagonists, or inhibitors of SECP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in. the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising SECP or fragments thereof. For example, liposome preparations containing a cell-impermeable macromolecule may promote cell fusion and intracellular delivery of the macromolecule. Alternatively, SECP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example SECP or fragments thereof, antibodies of SECP, and agonists, antagonists or inhibitors of SECP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of adrninistration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind SECP may be used for the diagnosis of disorders characterized by expression of SECP, or in assays to monitor patients being treated with SECP or agonists, antagonists, or inhibitors of SECP. Antibodies useful for diagnostic purposes may be prepared in the same manner as described above for therapeutics. Diagnostic assays for SECP include methods which utilize the antibody and a label to detect SECP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring SECP, including ELISAs, RIAs, and FACS, are known in the art and provide a basis for diagnosing altered or abnormal levels of SECP expression. Normal or standard values for SECP expression are established by combining body fluids or cell extracts taken from normal mammalian subjects, for example, human subjects, with antibodies to SECP under conditions suitable for complex formation. The amount of standard complex formation may be quantitated by various methods, such as photometric means. Quantities of SECP expressed in subject, control, and disease samples from biopsied tissues are compared with the standard values. Deviation between standard and subject values establishes the parameters for diagnosing disease.

In another embodiment of the invention, the polynucleotides encoding SECP may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of SECP may be correlated with disease. The diagnostic assay may be used to determine absence, presence, and excess expression of SECP, and to monitor regulation of SECP levels during therapeutic intervention.

In one aspect, hybridization with PCR probes which are capable of detecting polynucleotide sequences, including genomic sequences, encoding SECP or closely related molecules may be used to identify nucleic acid sequences which encode SECP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding SECP, allelic variants, or related sequences.

Probes may also be used for the detection of related sequences, and may have at least 50% sequence identity to any of the SECP encoding sequences. The hybridization probes of the subject invention may be DNA or RNA and may be derived from the sequence of SEQ ID NO:33-64 or from genomic sequences including promoters, enhancers, and introns of the SECP gene.

Means for producing specific hybridization probes for DNAs encoding SECP include the cloning of polynucleotide sequences encoding SECP or SECP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidinfbiotin coupling systems, and the like.

Polynucleotide sequences encoding SECP may be used for the diagnosis of disorders associated with expression of SECP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderrna, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigerninal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss. The polynucleotide sequences encoding SECP may be used in Southern or northern analysis, dot blot, or other membrane-based technologies; in PCR technologies; in dipstick, pin, and multiformat ELISA-like assays; and in microarrays utilizing fluids or tissues from patients to detect altered SECP expression. Such qualitative or quantitative methods are well known in the art.

In a particular aspect, the nucleotide sequences encoding SECP may be useful in assays that detect the presence of associated disorders, particularly those mentioned above. The nucleotide sequences encoding SECP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding SECP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of SECP, a normal or standard profile for expression is established. This may be accomplished by combining body fluids or cell extracts taken from normal subjects, either animal or human, with a sequence, or a fragment thereof, encoding SECP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or rnay provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding SECP may involve the use of PCR. These oligomers may be chemically synthesized, generated enzymatically, or produced in vitro. Oligomers will preferably contain a fragment of a polynucleotide encoding SECP, or a fragment of a polynucleotide complementary to the polynucleotide encoding SECP, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding SECP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding SECP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPS are also useful for examining differences in disease outcomes in monogenic disorders, such as cystic fibrosis, sickle cell anemia, or chronic granulomatous disease. For example, variants in the rmannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of SECP include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or calorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, SECP, fragments of SECP, or antibodies specific for SECP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for SECP to quantify the levels of SECP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding SECP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding SECP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, SECP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between SECP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with SECP, or fragments thereof, and washed. Bound SECP is then detected by methods well known in the art. Purified SECP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding SECP specifically compete with a test compound for binding SECP. In this manner, antibodies can be used to detect the presence of any peptide which shares one or more antigenic determinants with SECP.

In additional embodiments, the nucleotide sequences which encode SECP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/293,728, U.S. Ser. No. 60/297,019, U.S. Ser. No. 60/299,297, U.S. Ser. No. 60/300,537, U.S. Ser. No. 60/301,936, U.S. Ser. No. 60/366,041, U.S. Ser. No. 60/362,439, and U.S. Ser. No. 60/363,649, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitiogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S 1000, SEPHAROSE CL2B, or SEPHAROSE CLAB column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PcNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genormics), or derivatives thereof. Recombinant plasmids were transformed into competent E. coli cells including XL1-Blue, XLI-BlueMRF, or SOLR from Stratagene or DH5a, DH10B, or ElectroMAX DH10B from Invitrogen.

II. Isolation of cDNA Clones

Plasmids obtained as described in Example I were recovered from host cells by in vivo excision using the UNIAP vector system (Stratagene) or by cell lysis. Plasmids were purified using at least one of the following: a Magic or WIARD Minipreps DNA purification system (Promega); an AGTC Miniprep purification kit (Edge Biosystems, Gaithersburg Md.); and QIAWELL 8 Plasmid, QIAWELI 8 Plus Plasmnid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmnid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence. analysis systems known in the art. Reading frames within the cDNA sequences were identified using standard methods (reviewed in Ausubel, 1997, surra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norveicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MAcNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:33-64. Fragments from about 20 to about 4000 nucleotides which are useful in hybridization and amplification technologies are described in Table 4, column 2.

IV. Identification and Editing of Coding Sequences from Genomic DNA

Putative secreted proteins were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode secreted proteins, the encoded polypeptides were analyzed by querying against PFAM models for secreted proteins. Potential secreted proteins were also identified by homology to Incyte cDNA sequences that had been annotated as secreted proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example m. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genoric DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of SECP Encoding Polynucleotides

The sequences which were used to assemble SEQ ID NO:33-64 were compared with sequences from the Incyte LIFESEQ database and public domain databases using BLAST and other implementations of the Smith-Waterman algorithm. Sequences from these databases that matched SEQ ID NO:33-64 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Ginethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

In this manner, SEQ ID NO:50 was mapped to chromosome 14 within the interval from 59.0 to 68.0 centiMorgans.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding SECP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding SECP. cDNA sequences and cDNA library/tissue information are found in the LIFESEQ GOLD database (Incyte Genomics, Palo Alto Calif.).

VIII. Extension of SECP Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determiined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dirnethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomnic library.

IX. Identification of Single Nucleotide Polymorphisms in SECP Encoding Polynucleotides

Common DNA sequence variants known as single nucleotide polymorphisms (SNPs) were identified in SEQ ID NO:33-64 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:33-64 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N. H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample PreDaration

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 μg/el oligo-(dT) primer (21mer), IX first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5× SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymercoated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5× SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5× SSC in a corner of the chamber. The chamber containing the arrays is incubated for about 6.5 hours at 60° C. The arrays are washed for 10 min at 45° C. in a first wash buffer (1× SSC, 0.1% SDS), three times for 10 minutes each at 45° C. in a second wash buffer (0.1× SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

For example, SEQ ID NO:39 showed differential expression in inflammatory responses as determined by microarray analysis. The expression of SEQ ID NO:39 was decreased by at least two fold in an endothelial cell line treated with interleukin 1 (IL-1) and tumor necrosis factor (TNF). Therefore, SEQ ID NO:39 may be used in diagnostic assays for inflammatory responses.

SEQ ID NO:43 is co-expressed with one or more genes known to be involved in bone remodeling and osteoporosis. Therefore, SEQ ID NO:43 may be used in diagnostic assays for bone diseases such as osteoporosis.

Microarray analysis could be used, for example, to determine the expression levels of secreted proteins in T-cell leukemia cells following treatment with cell activators and ionophores. Based on this type of microarray analysis, it was determined that the expression of SEQ ID NO:48, is decreased at least 2-fold in Jurkat cells treated for one hour with 1 μM PMA and 50 ng/ml to 10 μg/ml of ionomycin, compared to untreated cells.

Microarray analysis could be used to determine the level of secreted proteins in differentiated adipocytes compared to undifferenciated preadipocytes. In one example, primary cultures of preadipocytes were obtained from subcutaneous fat from two female donors. Preadipocytes were cultured in F-10 medium with 10% fetal bovine serum. Confluent cells were either treated with 1 μM rosiglitazone (BRIA9653, a PPARγ agonist), 0.2 mM IBMX (3-isobutyl-1-methylxanthine, another inducer of adipocyte differentiation), and 100 nM human insulin for 3 days; or remained untreated. Thereafter, the cells were cultured in F-10 medium with 3% fetal bovine serum and 100 nM human insulin. Differentiated adipocytes were compared to untreated preadipocytes maintained in culture in the absence of inducing agents. Between 80% and 90% of the preadipocytes observed under a phase contrast microscope had differentiated to adipocytes by about two weeks. Cells were harvested at various times up to about two weeks following treatment (or mock treatment). Microarray analysis revealed that the expression of SEQ ID NO:44, which encodes the polypeptide of SEQ ID NO:12, is down-regulated between 5 and 7-fold in differentiated adipocytes compared to the undifferentiated cells. This down-regulation was observed from 1 day following treatment until the end of the experiment. Therefore, SEQ ID NO:48 has utility in monitoring cell proliferative diseases and cancers. SEQ ID NO:44 has utility in the monitoring and disease staging of lipid metabolism disorders.

In contrast, the expression of SEQ ID NO:44 was up-regulated about two-fold in a breast adenocarcinoma cell line (MCF7) following treatment with a selective, cell-permeable inhibitor of MAPK kinase/ERK kinase 1 (MEK1) that acts by inhibiting MAPK and the subsequent phosphorylation of MAPK substrates. In this experiment, cells were treated with 25 μM on the inhibitor for 8 days. Therefore SEQ ID NO:44 also has utility in monitoring aberrant cell proliferation or apoptosis resulting from GPCR-mediated signal transduction via the MAPK kinase and/or ERK kinase pathways.

Human peripheral blood mononuclear cells (PBMCs) can be classified into discrete cellular populations representing the major cellular component of the immune system. Expression of SEQ ID NO:57 was increased by at least 2-fold following exposure of these cells to (a) 0.1 μM/rnl PMA (a broad activator of protein kinase C) with 10 ng/ml ionomycin (a calcium ionophore that increases cytosolic calcium) for 20 h; and (b) 1 ng/ml SEB (a staphylococcal exotoxin, a specific activator of human T cells) for 72 h. Therefore, SEQ ID NO:57 may be used in diagnostic assays for inflammatory and immune responses.

Prostate tumor cell lines, LNCaP (human prostate carcinoma) and MDAPCa2b (human prostate adenocarcinoma), were grown by embedding single cell suspensions in Matrigel matrix. Matarigel matrix is a reconstituted basement membrane matrix isolated from a mouse sarcoma and composed of laminin, collagen IV, entactin, and heparin sulfate proteoglycan. It also contains growth factors, matrix metalloproteinases, and other components. Cells normally in contact with a basement membrane in vivo often are well differentiated when cultured on Matrigel basement membrane matrix in vitro. Understanding the contribution of the microenvironment to the development and metastasis of cancer and how to manipulate the interactions between cancer cells and the microenvironment, might lead to novel therapeutic targets. RNA from the prostate cancer cells was harvested when modestly sized colonies formed. SEQ ID NO:58 exhibited greater than a 2-fold increase in cDNA expression when these human prostate cancer cells were grown in Matrigel. Therefore, SEQ ID NO:58 may be used as a diagnostic marker or as a potential therapeutic target for prostate cancer.

As a further example, the expression of SEQ ID NO:62 was increased by at least two fold in LNCaP prostate carcinoma cells and MD)APCa2b prostate adenocarcinoma cells grown in single cell suspensions in Matrigel matrix relative to untreated cells. RNA was harvested when modestly sized colonies formed (i.e., the length of time required for normal epithelial cells undergo morphogenesis in the presence of Matrigel matrix). LNCaP prostate carcinoma cell line was isolated from a lymph node biopsy of a 50-year-old male with metastatic prostate carcinoma. LNCaP cells express prostate specific antigens, produce prostatic acid phosphatase, and express androgen receptors. MDAPCa2b prostate adenocarcinoma cell line was isolated from a metastatic site in the bone of a 63-year-old male. MDAPCa2b cell line expresses prostate specific antigen (PSA) and androgen receptor grows in vitro and in vivo and is androgen sensitive. This experiment showed that SEQ ID NO:62 may be used as a diagnostic marker or as a potential therapeutic target for cancers.

XII. Complementary Polynucleotides

Sequences complementary to the SECP-encoding sequences, or any parts thereof, are used to detect, decrease, or inhibit expression of naturally occurring SECP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of SECP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the SECP-encoding transcript.

XIII. Expression of SECP

Expression and purification of SECP is achieved using bacterial or virus-based expression systems. For expression of SECP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express SECP upon induction with isopropyl beta-D-thiogalactopyranoside (IPTG). Expression of SECP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding SECP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugirerda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, SECP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from SECP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified SECP obtained by these methods can be used directly in the assays shown in Examples XVII, and XVIII, where applicable.

XIV. Functional Assays

SECP function is assessed by expressing the sequences encoding SECP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of SECP on gene expression can be assessed using highly purified populations of cells transfected with sequences encoding SECP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding SECP and other genes of interest can be analyzed by northern analysis or microarray techniques.

XV. Production of SECP Specific Antibodies

SECP substantially purified using polyacrylamide gel electrophoresis (PAGE; see, e.g., Harrington, M. G. (1990) Methods Enzymol. 182:488495), or other purification techniques, is used to immunize animals (e.g., rabbits, mice, etc.) and to produce antibodies using standard protocols.

Alternatively, the SECP amino acid sequence is analyzed using LASERGENE software (DNASTAR) to determine regions of high imniunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, surra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, sunra.) Rabbits are immunized with the oligopeptide-KLH complex in complete Freund's adjuvant. Resulting antisera are tested for antipeptide and anti-SECP activity by, for example, binding the peptide or SECP to a substrate, blocking with 1% BSA, reacting with rabbit antisera, washing, and reacting with radio-iodinated goat anti-rabbit IgG.

XVI. Purification of Naturally Occurring SECP Using Specific Antibodies

Naturally occurring or recombinant SECP is substantially purified by immunoaffinity chromatography using antibodies specific for SECP. An immunoaffinity column is constructed by covalently coupling anti-SECP antibody to an activated chromatographic resin, such as CNBr-activated SEPHAROSE (Amersham Biosciences). After the coupling, the resin is blocked and washed according to the manufacturer's instructions.

Media containing SECP are passed over the immunoaffinity column, and the column is washed under conditions that allow the preferential absorbance of SECP (e.g., high ionic strength buffers in the presence of detergent). The column is eluted under conditions that disrupt antibody/SECP binding (e.g., a buffer of pH 2 to pH 3, or a high concentration of a chaotrope, such as urea or thiocyanate ion), and SECP is collected.

XVII. Identification of Molecules Which Interact with SECP

SECP, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled SECP, washed, and any wells with labeled SECP complex are assayed. Data obtained using different concentrations of SECP are used to calculate values for the number, affinity, and association of SECP with the candidate molecules.

Alternatively, molecules interacting with SECP are analyzed using the yeast two-hybrid system as described in Fields, S. and O. Song (1989) Nature 340:245-246, or using commercially available kits based on the two-hybrid system, such as the MATCHMAKER system (Clontech).

SECP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVII. Demonstration of SECP Activity

An assay for growth stimulating or inhibiting activity of SECP measures the amount of DNA synthesis in Swiss mouse 3T3 cells (McKay, I. and Leigh, I., eds. (1993) Growth Factors: A Practical Approach, Oxford University Press, New York, N.Y.). In this assay, varying amounts of SECP are added to quiescent 3T3 cultured cells in the presence of [³H]thymidine, a radioactive DNA precursor. SECP for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold SECP concentration range is indicative of growth modulating activity. One unit of activity per milliliter is defined as the concentration of SECP producing a 50% response level, where 100% represents maximal incorporation of [³H]thymidine into acid-precipitable DNA.

Alternatively, an assay for SECP activity measures the stimulation or inhibition of neurotransmission in cultured cells. Cultured CHO fibroblasts are exposed to SECP. Following endocytic uptake of SECP, the cells are washed with fresh culture medium, and a whole cell voltage-clamped Xenopus myocyte is manipulated into contact with one of the fibroblasts in SECP-free medium. Membrane currents are recorded from the myocyte. Increased or decreased current relative to control values are indicative of neuromodulatory effects of SECP (Morimoto, T. et al. (1995) Neuron 15:689-696).

Alternatively, an assay for SECP activity measures the amount of SECP in secretory, membrane-bound organelles. Transfected cells as described above are harvested and lysed. The lysate is fractionated using methods known to those of skill in the art, for example, sucrose gradient ultracentrifugation. Such methods allow the isolation of subcellular components such as the Golgi apparatus, ER, small membrane-bound vesicles, and other secretory organelles. Immunoprecipitations from fractionated and total cell lysates are performed using SECP-specific antibodies, and immunoprecipitated samples are analyzed using SDS-PAGE and immunoblotting techniques. The concentration of SECP in secretory organelles relative to SECP in total cell lysate is proportional to the amount of SECP in transit through the secretory pathway.

Alternatively, protease activity of SECP is measured by the hydrolysis of appropriate synthetic peptide substrates conjugated with various chromogenic molecules in which the degree of hydrolysis is quantified by spectrophotometric (or fluorometric) absorption of the released chromophore (Beynon, R. J. and J. S. Bond (1994) Proteolytic Enzymes: A Practical Approach, Oxford University Press, New York N.Y., pp.25-55). Peptide substrates are designed according to the category of protease activity as endopeptidase (serine, cysteine, aspartic proteases, or metalloproteases), aminopeptidase (leucine aminopeptidase), or carboxypeptidase (carboxypeptidases A and B, procollagen C-proteinase). Commonly used chromogens are 2-naphthylarine, 4-nitroaniline, and furylacrylic acid. Assays are performed at ambient temperature and contain an aliquot of the enzyme and the appropriate substrate in a suitable buffer. Reactions are carried out in an optical cuvette, and the increase/decrease in absorbance of the chromogen released during hydrolysis of the peptide substrate is measured. The change in absorbance is proportional to SECP activity in the assay.

Alternatively, AMP-binding activity of SECP is measured by combining SECP with ³²P-labeled AMP. The reaction is incubated at 37° C. and terminated by addition of trichloroacetic acid. The acid extract is neutralized and subjected to gel electrophoresis to remove unbound label. The radioactivity retained in the gel is proportional to SECP activity in the assay.

XVIII. Demonstration of Secretory Activity

Secretory activity can be quantified by comparing proteins secreted from [³⁵S]methionine-labeled cells grown at various temperatures and evaluated using SDS-PAGE. By labeling cells grown at 25, 37, and 39° C., the intensity of immunoprecipitated bands can be compared as a function of thermoregulation. Using this method, heat stress has been shown to increase secretion of a 150-kDa secretory glycoprotein in S. cerevisiae SEY2101a cells by 90% (Russo, P. et al. (1992) Proc. Natl. Acad. Sci. USA 89:3671-3675).

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Incyte | Polypeptide | Incyte | Polynucleotide | Incyte | Incyte Full |
|---|---|---|---|---|---|
| Project ID | SEQ ID NO: | Polypeptide ID | SEQ ID NO: | Polynucleotide ID | Length Clones |
| 2072638 | 1 | 2072638CD1 | 33 | 2072638CB1 | |
| 747515 | 2 | 747515CD1 | 34 | 747515CB1 | 1962430CA2 |
| 7495641 | 3 | 7495641CD1 | 35 | 7495641CB1 | 6552086CA2, |
| | | | | | 6748659CA2 |
| 2937340 | 4 | 2937340CD1 | 36 | 2937340CB1 | |
| 3765326 | 5 | 3765326CD1 | 37 | 3765326CB1 | |
| 948883 | 6 | 948883CD1 | 38 | 948883CB1 | 55137951CA2, |
| | | | | | 55137952CA2, |
| | | | | | 948883CA2 |
| 5665403 | 7 | 5665403CD1 | 39 | 5665403CB1 | |
| 7493065 | 8 | 7493065CD1 | 40 | 7493065CB1 | 3778951CA2, |
| | | | | | 6258260CA2 |
| 7493531 | 9 | 7493531CD1 | 41 | 7493531CB1 | 4371461CA2 |
| 3321454 | 10 | 3321454CD1 | 42 | 3321454CB1 | |
| 189299 | 11 | @189299CD1 | 43 | @189299CB1 | 90102151CA2, |
| | | | | | 90102267CA2 |
| 7488057 | 12 | 7488057CD1 | 44 | 7488057CB1 | |
| 7486411 | 13 | 7486411CD1 | 45 | 7486411CB1 | |
| 2005762 | 14 | 2005762CD1 | 46 | 2005762CB1 | 2329873CA2 |
| 2514091 | 15 | 2514091CD1 | 47 | 2514091CB1 | |
| 2726954 | 16 | 2726954CD1 | 48 | 2726954CB1 | 90093243CA2, |
| | | | | | 90097919CA2, |
| | | | | | 90097991CA2 |
| 5406015 | 17 | 5406015CD1 | 49 | 5406015CB1 | 5406015CA2 |
| 2850658 | 18 | 2850658CD1 | 50 | 2850658CB1 | |
| 6579653 | 19 | 6579653CD1 | 51 | 6579653CB1 | 6579653CA2 |
| 6819648 | 20 | 6819648CD1 | 52 | 6819648CB1 | 90088906CA2, |
| | | | | | 90088914CA2, |
| | | | | | 90088922CA2, |
| | | | | | 90088930CA2. |
| | | | | | 90088938CA2, |
| | | | | | 90088946CA2, |
| | | | | | 90089006CA2, |
| | | | | | 90089014CA2, |
| | | | | | 90089022CA2, |
| | | | | | 90089030CA2 |
| 2771521 | 21 | 2771521CD1 | 53 | 2771521CB1 | 8353114CA2 |
| 7095792 | 22 | 7095792CD1 | 54 | 7095792CB1 | 7095792CA2 |
| 7112696 | 23 | 7112696CD1 | 55 | 7112696CB1 | 7112696CA2 |
| 7759388 | 24 | 7759388CD1 | 56 | 7759388CB1 | 90105023CA2, |
| | | | | | 90105039CA2 |
| 8165414 | 25 | 8165414CD1 | 57 | 8165414CB1 | |
| 2540610 | 26 | 2540610CD1 | 58 | 2540610CB1 | 90099721CA2 |
| 1593380 | 27 | 1593380CD1 | 59 | 1593380CB1 | 3099733CA2 |
| 1480069 | 28 | 1480069CD1 | 60 | 1480069CB1 | 7077836CA2, |
| | | | | | 90143809CA2, |
| | | | | | 90143817CA2, |
| | | | | | 90143833CA2, |
| | | | | | 90143901CA2, |
| | | | | | 90143917CA2, |
| | | | | | 90143925CA2, |
| | | | | | 90143933CA2, |
| | | | | | 90143941CA2, |
| | | | | | 90171910CA2, |
| | | | | | 90172026CA2, |
| | | | | | 90172509CA2, |
| | | | | | 90172517CA2, |
| | | | | | 90172601CA2, |
| | | | | | 90172609CA2, |
| | | | | | 90172617CA2, |
| | | | | | 90172625CA2 |
| 2310442 | 29 | 2310442CD1 | 61 | 2310442CB1 | 3392596CA2, |
| | | | | | 90054767CA2, |
| | | | | | 90054851CA2, |
| | | | | | 90054903CA2, |
| | | | | | 90054911CA2, |
| | | | | | 90079371CA2 |
| 7503731 | 30 | 7503731CD1 | 62 | 7503731CB1 | |
| 7506368 | 31 | 7506368CD1 | 63 | 7506368CB1 | 90093259CA2 |
| 7509087 | 32 | 7509087CD1 | 64 | 7509087CB1 | |

TABLE 2

| | | GenBank ID NO: | | |
|---|---|---|---|---|
| Polypeptide | Incyte | or PROTEOME | Probability | |
| SEQ ID NO: | Polypeptide ID | ID NO: | Score | Annotation |
| 1 | 2072638CD1 | g3766136 | 0.0 | [Homo sapiens] Ig-like membrane protein |
| | | | | Saupe, S., Roizes, G., Peter, M., Boyle, S., Gardiner, |
| | | | | K. and De Sario, A. (1998) Molecular cloning of a |
| | | | | human CDNA IGSF3 encoding an immunoglobulin-like |
| | | | | membrane protein: expression and mapping to chromosome |
| | | | | band 1p13 |
| | | | | Genomics 52:305-311 |
| 5 | 3765326CD1 | g6841342 | 4.7E−62 | [Homo sapiens] HSPC052 |
| | | | | Zhang, Q. H., et al. (2000) Cloning and functional |
| | | | | analysis of CDNAs with open reading frames for 300 |
| | | | | previously undefined genes expressed in CD34+ |
| | | | | hematopoietic stem/progenitor cells. |
| | | | | Genome Res. 10, 1546-1560 |
| 10 | 3321454CD1 | g14575530 | 0.0 | [Homo sapiens] leishmanolysin-like peptidase, |
| | | | variant 1 | |
| | | g8101109 | 1.9E−19 | ]Trypanosoma cruzi] GP63-4 protein |
| | | | | Grandgenett, P. M., et al. (2000) Differential |
| | | | | expression of GP63 genes in Trypanosoma cruzi. |
| | | | | Mol. Biochem. Parasitol. 110, 409-415 |
| 11 | 189299CD1 | g4239895 | 3.0E−20 | ]Homo sapiens] MASL1 |
| | | | | Sakabe, T., et al. (1999) Identification of a novel |
| | | | | gene, MASL1, within an amplicon at 8p23.1 detected in |
| | | | | malignant fibrous histiocytomas by comparative genomic |
| | | | | hybridization. |
| | | | | Cancer Res. 59, 511-515 |
| 12 | 7488057CD1 | g609314 | 6.0E−229 | [Homo sapiens] pregnancy-specific beta 1-glycoprotein |
| | | | | 7 precursor Teglund, S. et al. (1995) Characterization |
| | | | | of CDNA encoding novel pregnancy-specific glycoprotein |
| | | | | variants. Biochem. Biophys. Res. Commun. 211:656-664 |
| 13 | 7486411CD1 | g6650760 | 1.3E−50 | [Homo sapiens] placental protein 13; PP13 |
| | | | | Bohn, H. et al. (1983) Purification and characteri- |
| | | | | zation of two new soluble placental tissue proteins |
| | | | | (PP13 and PP17). Oncodev. Biol. Med. 4:343-350 |
| 25 | 8165414CD1 | g4579909 | 0.0 | [Homo sapiens] apg-2 |
| | | | | Nonoguchi, K. (1999) Cloning of human CDNAs for Apg-1 |
| | | | | and Apg-2, members of the Hsp110 family, and chromo- |
| | | | | somal assignment of their genes Gene 237:21-28 |
| 31 | 7506368CD1 | g14270766 | 8.7E−27 | [Homo sapiens] putative TCPTP(T-cell tyrosine |
| | | | | phosphatase)-interacting protein |

TABLE 3

| | | | | |
|---|---|---|---|---|
| SEQ | Incyte | Amino | | |
| ID | Polypeptide | Acid | | Analytical Methods |
| NO: | ID | Residues | Signature Sequences, Domains and Motifs | and Databases |

| | | | | |
|---|---|---|---|---|
| 1 | 2072638CD1 | 934 | Immunoglobulin domain: G34-V117, G164-V252, K706-V822, T570- | HMMER_PFAM |
| | | | V659, S298-A386, N433-V523 | |
| | | | Transmembrane domian: A864-R892 | TMAP |
| | | | PROTEIN PROSTAGLANDIN F2-ALPHA RECEPTOR REGULATORY PRECURSOR | BLAST_PRODOM |
| | | | ASSOCIATED SIGNAL IMMUNOGLOBULIN FOLD PD025644: V22-S841 | |
| | | | LEUKOCYTE SURFACE PROTEIN PD044106: L407-V549, F782-1909 | BLAST_PRODOM |
| | | | IMMUNOGLOBULIN DM00001\|I39207\|816-919: S422-L528 | BLAST_DOMO |
| | | | Potential Phosphorylation Sites: S6 S110 S133 S172 S213 S240 | MOTIFS |
| | | | S272 S351 S375 S424 S441 S462 S476 S542 S566 S704 S719 S731 | |
| | | | S742 S787 S811 S894 T2 T16 T21 T26 T32 T118 T278 T296 T320 | |
| | | | T356 T429 T482 T548 T702 T768 T837 T919 Y113 Y248 | |
| | | | Potential Glycosylation Sites: N60 N157 N394 N439 N564 N581 | MOTIFS |
| | | | N700 N817 N893 | |
| 2 | 747515CD1 | 236 | Signal_cleavage: M1-G33 | SPSCAN |
| | | | Transforming protein P21 RAS signature PR00449: V7-Q28, G53- | BLIMPS_PRINTS |
| | | | 575, N138-Q151, Y185-R207 | |
| | | | RAS TRANSFORMING PROTEIN | BLAST_DOMO |
| | | | DM00006\|P34143\|3-150: K8-R116, D131-K156 | |
| | | | DM00006\|Q05737\|5-150: K8-R116, E133-A165 | |
| | | | DM00006\|S41430\|5-150; K8-R116, E133-E169 | |
| | | | DM00006\|P36018\|1-174: K8-R116, Q132-E159 | |
| | | | ATP/GTP-binding site motif A (P-loop): G13-S20 | MOTIFS |
| | | | Sigma-54 interaction domain ATP-binding region A signature: | MOTIFS |
| | | | V9-L22 | |
| | | | Potential Phosphorylation Sites: S76 S79 S103 S110 T54 T98 | MOTIFS |
| | | | T126 T147 T155 T229 Y208 | |
| 3 | 7495641CD1 | 171 | Signal_cleavage: M1-A16 | SPSCAN |
| | | | Potential Phosphorylation Sites: S28 T64 | MOTIFS |
| 4 | 2937340CD1 | 316 | Signal_cleavage: M1-G42 | SPSCAN |
| | | | Potential Phosphorylation Sites: S79 S149 S153 S237 S307 T166 | MOTIFS |
| | | | T195 T199 | |
| | | | Potential Glycosylation Sites: N96 | MOTIFS |
| 5 | 3765326CD1 | 513 | Signal_cleavage: M1-Q54 | SPSCAN |
| | | | Transmembrane domains: N166-R194, K308-1324 | TMAP |
| | | | ATP/GTP-binding site motif A (P-loop): A344-T351 | MOTIFS |
| | | | N-6 Adenine-specific DNA methylases signature: M273-F279 | MOTIFS |
| | | | Potential Phosphorylation Sites: S23 S158 S302 S306 S356 S464 | MOTIFS |
| | | | S479 T58 T120 T200 T254 T289 T351 T401 T457 T463 T486 T498 | |
| | | | Potential Glycosylation Sites: N148 N493 | MOTIFS |
| 6 | 948883CD1 | 123 | Signal_cleavage: M1-G44 | SPSCAN |
| | | | Signal Peptide: M1-A16 | HMMER |
| | | | Signal Peptide: M9-G44 | HMMER |
| | | | Potential Phosphorylation Sites: S20 S45 S102 T41 | MOTIFS |
| | | | Potential Glycosylation Sites: N81 | MOTIFS |
| 7 | 5665403CD1 | 125 | Signal_cleavage: M1-T39 | SPSCAN |
| | | | Transmembrane domain: L20-S37; N-terminus is non-cytosolic | TMAP |
| | | | Potential Phosphorylation Sites: S23 S112 Y17 | MOTIFS |
| 8 | 7493065CD1 | 267 | Signal_cleavage: M1-N38 | SPSCAN |
| | | | Potential Phosphorylation Sites: S191 S253 T73 T86 | MOTIFS |
| 9 | 7493531CD1 | 71 | Signal_cleavage: M1-G44 | SPSCAN |
| | | | Potential Phosphorylation Sites: S52 | MOTIFS |
| 10 | 3321454CD1 | 642 | Signal_cleavage: M1-A43 | SPSCAN |
| | | | Leishmanolysin domain: E196-G398, V425-P594 | HMMER_PFAM |
| | | | Transmembrane domain: W23-S42, T601-L625; N-terminus is | TMAP |
| | | | cytosolic | |
| | | | Leishmanolysin (M8) metalloprotease family signature PR00782: | BLIMPS_PRINTS |
| | | | V327-Q356, G358-L386 | |
| | | | SURFACE GLYCOPROTEIN PRECURSOR SIGNAL PROTEASE MAJOR GP63 | BLAST_PRODOM |
| | | | LEISHMANOLYSIN HYDROLASE PROMASTIGOTE PD003085: Y296-G398, | |
| | | | E196-F272, Y405-F470, 595-G130 | |
| | | | LEISHMANOLYSIN DM03460\|Q06031\|1-614: T326-G398, G188-F272, | BLAST_DOMO |
| | | | D396-F470, E102-R136, C537-E590 | |
| | | | Potential Phosphorylation Sites: S62 S95 S117 S199 S304 S529 | MOTIFS |
| | | | S584 T90 T212 T261 T284 T332 T397 T408 T417 Y153 | |
| | | | Potential Glycosylation Sites: N295 N602 | MOTIFS |
| 11 | 189299CD1 | 277 | Signal_cleavage: M1-C17 | |
| | | | Leucine Rich Repeat: E53-Q75, E168-P190, N122-S144, Q99-Q121, | HMMER_PFAM |
| | | | F191-S212, N76-K98, L145-Q167 | |
| | | | Leucine-rich repeat signature PR00019: L97-L110, L54-L67 | BLIMPS_PRINTS |
| | | | Potential Phosphorylation Sites: S50 S201 S213 T4 T96 | MOTIFS |
| 12 | 7488057CD1 | 419 | Signal cleavage: M1-A34 | SPSCAN |
| | | | Signal Peptide: M1-A34 | HMMER |
| | | | Immunoglobulin domain: G347-V396, K255-I312, T63-T121, T162- | HMMER-PFAM |
| | | | I219 | |
| | | | PRECURSOR GLYCOPROTEIN SIGNAL PREGNANCY-SPECIFIC | BLAST-PRODOM |
| | | | CARCINOEMBRYONIC ANTIGEN IMMUNOGLOBULIN FOLD PREGNANCY | |
| | | | MULTIGENE: PD000677: N29-T113; PD002253: P147-E218, P240-E311 | |
| | | | CARCINOEMBRYONIC ANTIGEN PRECURSOR AMINO-TERMINAL DOMAIN | BLAST-DOMO |
| | | | DM00372\|A54312\|38-148: I38-P149 | |
| | | | IMMUNOGLOBULIN: DM00001\|P11462\|236-320: L236-S321 | BLAST-DOMO |
| | | | Cell attachment sequence: R127-D129 | MOTIFS |
| | | | Actinin-type actin-binding domain signature 1: E267-N276 | MOTIFS |
| | | | Potential Phosphorylation Sites: S96 S226 S283 S395 S405 T14 | MOTIFS |
| | | | T113 T135 T146 T198 T301 T385 T401 T410 Y351 | |
| | | | Potential Glycosylation Sites: N61 N104 N111 N199 N209 N259 | MOTIFS |
| | | | N268 N303 | |
| 13 | 7486411CD1 | 142 | Signal cleavage: M1-N32 | SPSCAN |
| | | | Vertebrate galactoside-binding lectin: S30-D129 | HMMER-PFAM |
| | | | Vertebrate galactoside-b: BL00309: R115-N139, N45-G59, | BLIMPS-BLOCKS |
| | | | V62-G86 | |
| | | | Vertebrate galactoside-binding lectin signature: P34-K103 | PROFILESCAN |
| | | | VERTEBRATE GALACTOSIDE-BINDING LECTIN: | BLAST_DOMO |
| | | | DM00426\|Q05315\|1-136: S2-L132 | |
| | | | DM00426\|P38552\|14-148: T5-V135 | |
| | | | DM00426\|A55664\|14-148: T5-V135 | |
| | | | DM00426\|A55975\|13-150: T5-V135 | |
| | | | Potential Phosphorylation Sites: S30 T25 Y94 Y110 | MOTIFS |
| 14 | 2005762CD1 | 119 | Signal cleavage: M1-S18 | SPSCAN |
| | | | Signal Peptide: M1-S18 | HMMER |
| | | | Transmembrane domain: Y20-T44 | TMAP |
| | | | Potential Phosphorylation Sites: S51 T14 | MOTIFS |
| | | | Potential Glycosylation Sites: N73 | MOTIFS |
| 15 | 2514091CD1 | 249 | Signal Cleavage: M1-A53 | SPSCAN |
| | | | Signal Peptide: M35-A53 | HMMER |
| | | | Transmembrane domain: L26-E54; N-terminus is non-cytosolic | TMAP |
| | | | Potential Phosphorylation Sites: S9 S66 S168 S215 T87 T104 | MOTIFS |
| | | | T141 Y96 | |
| 16 | 2726954CD1 | 314 | Signal Cleavage: M1-T67 | SPSCAN |
| | | | Transmembrane domain: C37-F65; N-terminus is non-cytosolic | TMAP |
| | | | Potential Phosphorylation Sites: S135 S163 S239 S309 S311 T80 | MOTIFS |
| | | | T99 T134 T217 T293 | |
| 17 | 5406015CD1 | 183 | Signal Peptide: M4-A31, M4-S26 | HMMER |
| | | | Transmembrane domain: M4-F20; N-terminus is non-cytosolic | TMAP |
| | | | Leucine zipper pattern: L36-L57 | MOTIFS |
| | | | Potential Phosphorylation Sites: S19 S74 S124 S169 T98 | MOTIFS |
| 18 | 2850658CD1 | 621 | Signal cleavage: M1-G20 | SPSCAN |
| | | | Signal Peptide: M1-G20 | HMMER |
| | | | 3′-5′ exonuclease: D101-S263 | HMMER-PFAM |
| | | | Transmembrane domain: S2-R30, I241-N265; N-terminus is non- | TMAP |
| | | | cytosolic | |
| | | | Cytochrome c family heme-binding site signature: C459-A464 | MOTIFS |
| | | | Potential Phosphorylation Sites: S34 S69 S97 S206 S209 S228 | MOTIFS |
| | | | S266 S276 S320 S348 S407 S428 T151 T234 T517 Y191 | |
| 19 | 6579653CD1 | 79 | Signal cleavage: M1-L25 | SPSCAN |
| | | | Signal Peptide: M1-L25 | HMMER |
| | | | Transmembranedomain: L3-R31; N-terminus is non-cytosolic | TMAP |
| | | | Potential Phosphorylation Sites: S45 T33 T57 | MOTIFS |
| | | | Potential Glycosylation Sites: N6 | MOTIFS |
| 20 | 16819648CD1 | 83 | Signal_cleavage: M1-R20 | SPSCAN |
| | | | Signal Peptide: M1-A30 | HMMER |
| | | | Signal Peptide: M1-R20 | HMMER |
| | | | Signal Peptide: M1-R28 | HMMER |
| 21 | 2771521CD1 | 204 | Signal_cleavage: M1-W54 | SPSCAN |
| | | | Potential Phosphorylation Sites: S57 S152 | MOTIFS |
| 22 | 7095792CD1 | 83 | Signal_cleavage: M1-S17 | SPSCAN |
| | | | Signal Peptide: M1-A22 | HMMER |
| | | | Signal Peptide: M1-517 | HMMER |
| | | | Transmembrane domain: Q16-A44; N-terminus is non-cytosolic | TMAP |
| | | | DEAD and DEAH box families ATP-dependent helicases | PROFILESCAN |
| | | | signatures: F21-R72 | |
| | | | Potential Phosphorylation Sites: S47 | MOTIFS |
| 23 | 711269CD1 | 174 | Signal_cleavage: M1-G24 | SPSCAN |
| | | | Signal Peptide: M1-G24 | HMMER |
| | | | Transmembrane domain: I4-F27; N-terminus is non-cytosolic | TMAP |
| | | | Potential Phosphorylation Sites: S36 S48 S70 S81 S159 S165 | MOTIFS |
| | | | T56 T140 T169 | |
| | | | Potential Glycosylation Sites: N68 N168 | MOTIFS |
| 24 | 7759388CD1 | 771 | Signal_cleavage: M1-G31 | SPSCAN |
| | | | Signal Peptide: M1-G31 | HMMER |
| | | | Signal Peptide: S12-G31 | HMMER |
| | | | Signal Peptide: S8-G31 | HMMER |
| | | | Leucine Rich Repeat: N138-L161, T163-V186, K211-R233, A114- | HMMER_PFAM |
| | | | G137, R66-T89, S90-R113, N187-H210 | |
| | | | Leucine rich repeat C-terminal domain: N252-E297 | HMMER_PFAM |
| | | | Fibronectin type III domain: S422-T502 | HMMER_PFAM |
| | | | Immunoglobulin domain: G314-A372 | HMMER_PFAM |
| | | | Transmembrane domain: L10-W25, H530-R558 | TMAP |
| | | | Potential Phosphorylation Sites: S122 S162 S219 S288 S318 | MOTIFS |
| | | | S344 S422 S460 S461 S567 S659 S753 T163 T267 T282 T361 T517 | |
| | | | T658 T726 T761 Y710 | |
| | | | Potential Glycosylation Sites: N87 N343 N420 N459 | MOTIFS |
| 25 | 8165414CD1 | 841 | Signal_cleavage: M1-A20 | SPSCAN |
| | | | Hsp79protein: V3-Q607 | HMMER_PFAM |
| | | | Heat shock hsp70 proteins family proteins BL00297: P457-M510, | BLIMPS_BLOCKS |
| | | | V4-S40, P43-191, V132-I183, V198-D237, D296-E339, G359-A386, | |
| | | | D396-Q450 | |
| | | | 70 Kd heat shock protein signature PR00301: S2-V15, A52-A60, | BLIMPS_PRINTS |
| | | | C140-T160, I335-K351, L366-A386 | |
| | | | HEAT SHOCK PROTEIN ATP-BINDING FAMILY MULTIGENE CHAPERONE | BLAST_PRODOM |
| | | | HSP70 DNA K PRECURSOR PD000089: V3-E627 | |
| | | | PROTEIN HEAT SHOCK 01 03 02 CG8 CG4 CG3 CG9 PD152186: | BLAST_PRODOM |
| | | | Y624-I712 | |
| | | | ATP-BINDING PROTEIN HEAT SHOCK SPERM HEAT SHOCK MULTIGENE | BLAST_PRODOM |
| | | | FAMILY RECEPTOR EGG PD005795: F689-S784 | |
| | | | PROTEIN HEAT SHOCK 70-RELATED APG2 AT-BINDING MULTIGENE | BLAST_PRODOM |
| | | | FAMILY ISCHEMIA RESPONSIVE PD109124: P786-D841 | |
| | | | HEAT SHOCK HSP70 PROTEINS FAMILY DM00029 | BLAST_DOMO |
| | | | \|P34932\|1-390: M1-E391 | |
| | | | \|I56208\|1-390: M1-E391 | |
| | | | \|P48722\|1-390: M1-E391 | |
| | | | \|A57513\|1-390: M1-E391 | |
| | | | Hsp70_2 F200-C213 | MOTIFS |
| | | | Hsp70_3 V338-E352 | MOTIFS |
| | | | Potential Phosphorylation Sites: S31 S155 S323 S393 S403 S408 | MOTIFS |
| | | | S415 S448 S475 S496 S575 S641 S715 S737 S831 T104 T149 T225 | |
| | | | T538 T649 T732 T832 Y336 Y626 Y723 | |
| | | | Potential Glycosylation Sites: N45 N111 N172 N280 | MOTIFS |
| 26 | 2540610CD1 | 394 | Signal_cleavage: M1-A48 | SPSCAN |
| | | | Signal Peptide: M17-A48 | HMMER |
| | | | Transmembrane domain: S27-A47 Transmembrane segments: | TMAP |
| | | | N-terminus is non-cytosolic | |
| | | | Potential Phosphorylation Sites: S64 S69 S96 S112 S203 S252 | MOTIFS |
| | | | S308 S325 S361 S374 T18 T285 T355 | |
| 27 | 1593380CD1 | 196 | Signal_cleavage: M1-G22 | SPSCAN |
| | | | Signal Peptide: M1-A20 | HMMER |
| | | | Signal Peptide: M1-G22 | HMMER |
| | | | SignaI Peptide: M1-C23 | HMMER |
| | | | Leucine Rich Repeat: A107-P130, G83-A106, Q155-P178, R131- | HMMER_PFAM |
| | | | A154 | |
| | | | Transmembrane domain: M1-C29: Transmembrane segments; | TMAP |
| | | | N-terminus is non-cytosolic | |
| | | | Potential Phosphorylation Sites: S117 S139 | MOTIFS |
| | | | Potential Glycosylation Sites: N115 | MOTIFS |
| 28 | 1480069CD1 | 87 | Signal_cleavage: M1-G16 | SPSCAN |
| | | | SignalPeptide: M1-G16 | HMMER |
| | | | Potential Phosphorylation Sites: S41 S46 T17 | MOTIFS |
| 29 | 2310442CD1 | 233 | Signal_cleavage: M1-G16 | SPSCAN |
| | | | Signal Peptide: M1-G16 | HMMER |
| | | | Signal Peptide: M1-S20 | HMMER |
| | | | Signal Peptide: M1-T24 | HMMER |
| | | | Immunoglobulins and major histocompatibility complex | PROFILESCAN |
| | | | proteins: N191-S233 | |
| | | | Immunoglobulin domain: G34-V108, A148-V216 | HMMER_PFAM |
| | | | Immunoglobulins and major histocompatibility | BLIMPS_BLOCKS |
| | | | complex proteins BL00290: T152-S174, Y212- | |
| | | | 8HS20 PROTEIN PRECURSOR PD174509: L23-V108 | BLAST_PRODOM |
| | | | FRAMEWORK DOMAIN DM00397\|S24319\|1-128: M1-P130 | BLAST_DOMO |
| | | | IMMUNOGLOBULIN; IG; HISTOCOMPATIBILITY; MAJOR; DM02680\| | BLAST_DOMO |
| | | | A39949\|1-118: S116-C232 | |
| | | | FRAMEWORK DOMAIN DM00397\|P01719\|1-107: Y21-G 128 | BLAST_DOMO |
| | | | FRAMEWORK DOMAIN DM00397\|S30526\|1-119: S20-F139 | BLAST_DOMO |
| | | | Ig_Mhc Y212-H218 | MOTIFS |
| | | | Potential Phosphorylation Sites: S70 S94 S111 S142 S208 | MOTIFS |
| | | | S221 T36 | |
| 30 | 7503731CD1 | 373 | Signal_cleavage: M1-A48 | SPSCAN |
| | | | Signal Peptide: M17-A48 | HMMER |
| | | | Potential Phosphorylation Sites: S64 S69 S96 S112 S203 S252 | MOTIFS |
| | | | S266 S279 S310 T18 | |
| 31 | 7506368CD1 | 301 | Signal_cleavage: M1-T67 | SPSCAN |
| | | | Potential Phosphorylation Sites: S135 S163 S239 S296 S298 T80 | MOTIFS |
| | | | T99 T134 T217 T280 | |
| | | | Potential Glycosylation Sites: N157 | MOTIFS |
| 32 | 7509087CD1 | 193 | Potential Phosphorylation Sites: S57 | MOTIFS |

TABLE 4

| Polynucleotide | |
|---|---|
| SEQ ID NO:/ | |
| Incyte ID/ | |
| Sequence Length | Sequence Fragments |
| 33/2072638CB1/3716 | 1-530, 4-492, 30-252, 110-388, |
| | 166-434, 192-824, 205-511, 234- |
| | 587, 287-502, 369-617, 369-729, |
| | 369-1006, 622-884, 627-929, 674- |
| | 1120, 886-1433, 930-1418, 945- |
| | 1527, 956-1370, 965-1314, 965- |
| | 1536, 1009-1509, 1013-1700, |
| | 1033-1177, 1076-1646, 1130-1781, |
| | 1170-1863, 1223-1727, 1248-1867, |
| | 1249-1778, 1277-1851, 1286-1863, |
| | 1327-1690, 1331-1922, 1368-1897, |
| | 1382-1813, 1407-2092, 1413-2068, |
| | 1479-2079, 1533-1938, 1573-2130, |
| | 1621-2140, 1625-1933, 1625-2080, |
| | 1625-2093, 1625-2114, 1630-2306, |
| | 1631-1670, 1631-1679, 1631-1707, |
| | 1643-1677, 1648-1677, 1650-1884, |
| | 1663-2218, 1686-2176, 1734-2193, |
| | 1735-2360, 1744-2315, 1872-2363, |
| | 1882-2029, 1905-2071, 1971-2456, |
| | 1974-2514, 1975-2175, 2003-2269, |
| | 2016-2377, 2020-2188, 2034-2622, |
| | 2039-2087, 2039-2115, 2056-2085, |
| | 2056-2115, 2096-2717, 2148-2660, |
| | 2183-2651, 2437-3025, 2470-2668, |
| | 2476-3017, 2486-3039, 2488-2743, |
| | 2491-3092, 2534-2574, 2539-2574, |
| | 2540-2574, 2543-3052, 2544-2574, |
| | 2545-2569, 2545-2574, 2581-3246, |
| | 2590-3043, 2603-3161, 2672-3193, |
| | 2674-3163, 2675-3312, 2728-2897, |
| | 2750-2972, 2801-3444, 2858-3021, |
| | 2876-3449, 2897-3412, 2956-3462, |
| | 2965-3243, 2970-3117, 2970-3410, |
| | 2979-3248, 3130-3428, 3151-3425, |
| | 3171-3716, 3173-3449, 3213-3407, |
| | 3272-3428, 3274-3568, 3310-3595, |
| | 3316-3580, 3322-3456, 3461-3716 |
| 34/747515CB1/2398 | 1-235, 1-490, 5-548, 6-418, 8- |
| | 280, 8-3 18, 8-676, 15-455, 20- |
| | 170, 20-270, 20-488, 20-576, 23- |
| | 249, 23-275, 30-373, 30-705, 31- |
| | 294, 33-255, 33-263, 33-561, 33- |
| | 749, 35-546, 38-483, 38-649, 49- |
| | 524, 266-726, 384-947, 385-537, |
| | 385-677, 385-944, 399-1080, 430- |
| | 981, 568-1004, 580-1003, 581-836, |
| | 581-1086, 583-1003, 598-1003, |
| | 604-4173, 607-933, 634-1004, |
| | 658-1107, 682-967, 685-1004, |
| | 688-879, 714-882, 714-1009, 740- |
| | 1362, 806-1066, 835-1107, 849- |
| | 1080, 918-1167, 1072-1620, 1234- |
| | 1504, 1327-1903, 1358-2005, |
| | 1413-1579, 1650-2134, 1678-1903, |
| | 1678-1922, 1721-1952, 1891-2359, |
| | 1936-2178, 1981-2126, 1981-2127, |
| | 1981-2250, 2052-2323, 2052-2325, |
| | 2052-2398, 2116-2398 |
| 35/7495641CB1/2533 | 1-443, 1-566, 1-655, 5-640, 37- |
| | 441, 143-475, 248-705, 293-976, |
| | 533-1142, 596-1140, 621-1250, |
| | 675-1361, 693-1362, 695-1362, |
| | 721-1324, 732-1354, 747-1239, |
| | 758-1303, 758-1323, 758-1417, |
| | 763-933, 768-1039, 1016-1221, |
| | 1115-1307, 1146-1356, 1174-1637, |
| | 1215-1863, 1311-1921, 1312-1716, |
| | 1357-1611, 1357-1622, 1357-1668, |
| | 1357-1720, 1357-1734, 1357-1869, |
| | 1357-1899, 1357-1931, 1357-1997, |
| | 1376-1668, 1376-1711, 1419-1599, |
| | 1519-2028, 1529-1761, 1557-1996, |
| | 1565-1936, 1565-1995, 1593-1847, |
| | 1593-1852, 1599-1761, 1600-1869, |
| | 1615-1965, 1657-2277, 1667-2194, |
| | 1712-2284, 1714-2324, 1729-1966, |
| | 1734-2355, 1751-1998, 1751-2376, |
| | 1762-1965, 1764-2336, 1771-1964, |
| | 1772-2319, 1774-2382, 1837-2440, |
| | 1870-2168, 1899-2450, 1925-2533, |
| | 1966-2168, 1966-2232, 2000-2168, |
| | 2066-2209, 2077-2232 |
| 36/2937340CB1/1424 | 1-356, 28-244, 69-304, 69-600, |
| | 377-655, 436-661, 450-700, 458- |
| | 886, 458-1061, 462-812, 516-777, |
| | 516-924, 653-1192, 691-959, 691- |
| | 960, 691-972, 740-931, 759-1367, |
| | 760-918, 760-1023, 796-1337, |
| | 796-1376, 862-1389, 911-1424, |
| | 924-1075, 960-1410, 977-1178, |
| | 987-1410, 1003-1228, 1003-1363, |
| | 1005-1367, 1009-1421, 1029-1410, |
| | 1040-1410, 1045-1176, 1045-1219, |
| | 1045-1291, 1045-1369, 1045-1387, |
| | 1045-1395, 1045-1396, 1045-1409, |
| | 1045-1417, 1045-1419, 1045-1420, |
| | 1045-1421, 1045-1422, 1045-1423, |
| | 1045-1424, 1049-1410, 1065-1212, |
| | 1073-1410, 1080-1329, 1080-1424, |
| | 1082-1310, 1102-1406, 1107-1200, |
| | 1113-1410, 1118-1261, 1132-1409, |
| | 1179-1360, 1179-1424, 1255-1418, |
| | 1270-1406 |
| 37/3765326CB1/2448 | 1-780, 6-7 15, 9-552, 10-736, |
| | 11-716, 18-859, 20-246, 20-510, |
| | 26-683, 28-280, 45-279, 75-334, |
| | 75-454, 144-522, 388-1167, 440- |
| | 1103, 517-1103, 620-1332, 622- |
| | 1237, 711-1029, 775-927, 775- |
| | 1028, 775-1227, 928-1150, 1027- |
| | 1114, 1114-1506, 1125-1589, |
| | 1149-1442, 1149-1698, 1149-1855, |
| | 1153-1861, 1154-1636, 1223-1846, |
| | 1292-1551, 1298-1608, 1299-1595, |
| | 1387-1762, 1410-2061, 1567-1850, |
| | 1711-2295, 1876-2448, 1883-2447, |
| | 1883-2448, 1990-2265, 1994-2359, |
| | 2097-2351, 2100-2394, 2111-2434, |
| | 2129-2424, 2129-2435, 2161-2448, |
| | 2250-2429, 2250-2448, 2264-2429 |
| 38/948883CB1/1616 | 1-242, 1-371, 1-379, 1-380, 1- |
| | 398, 1-405, 1-407, 1-408, 1-413, |
| | 1-414, 1-415, 1-425, 1-431, 1- |
| | 433, 1-437, 1-440, 1-441, 1-442, |
| | 1-450, 1-454, 1-465, 1-466, 1- |
| | 472, 1-489, 1-494, 1-522, 1-531, |
| | 1-569, 1-570, 1-585, 1-605, 1- |
| | 622, 1-623, 1-63 1, 1-636, 1- |
| | 638, 1-640, 1-645, 1-647, 1-648, |
| | 1-655, 1-656, 1-663, 1-667, 1- |
| | 676, 1-685, 1-698, 1-724, 1-725, |
| | 1-749, 1-764, 1-775, 2-719, 2- |
| | 823, 3-464, 3-639, 3-678, 4-607, |
| | 4-668, 4-765, 4-769, 4-779, 12- |
| | 420, 20-717, 37-795, 38-496, 68- |
| | 633, 90-794, 91-576, 107-861, |
| | 130-867, 137-636, 140-634, 146- |
| | 886, 168-658, 183-809, 186-933, |
| | 196-647, 197-766, 205-769, 219- |
| | 657, 219-680, 221-684, 225-763, |
| | 230-833, 250-775, 251-794, 251- |
| | 1158, 253-802, 262-815, 263-826, |
| | 266-794, 270-772, 280-971, 310- |
| | 1025, 312-602, 318-841, 329- |
| | 1168, 340-858, 345-1019, 367- |
| | 808, 368-841, 371-894, 373-831, |
| | 383-1092, 385-893, 387-878, 391- |
| | 853, 397-1048, 399-831, 401- |
| | 1062, 418-1228, 419-923, 423- |
| | 952, 429-1033, 434-1024, 452- |
| | 888, 459-1039, 464-1025, 466- |
| | 928, 469-992, 478-947, 479-975, |
| | 482-984, 485-1008, 485-1169, |
| | 489-992, 489-1020, 499-1086, |
| | 527-834, 527-1066, 528-944, 529- |
| | 1092, 530-988, 543-1036, 555- |
| | 1044, 580-1269, 584-1041, 587- |
| | 1094, 589-1212, 590-1257, 593- |
| | 1237, 596-941, 598-1039, 598- |
| | 1105, 599-1055, 600-1093, 612- |
| | 1276, 613-1289, 614-802, 628- |
| | 1112, 631-1190, 631-1195, 635- |
| | 1353, 636-1155, 637-1074, 637- |
| | 1284, 639-1155, 640-1195, 641- |
| | 1210, 644,1336, 650-1187, 681- |
| | 1215, 688-1280, 689-1153, 691- |
| | 1136, 694-1199, 698-1176, 705- |
| | 1437, 709-1247, 710-1222, 730- |
| | 1286, 730-1377, 735-1261, 745- |
| | 1399, 756-1242, 758-1295, 763- |
| | 1243, 763-1441, 763-1456, 780- |
| | 1180, 786-1610, 794-1284, 794- |
| | 1494, 799-1616, 802-1616, 807- |
| | 1337, 810-1610, 810-1616, 831- |
| | 1616, 832-1517, 836-1332, 838- |
| | 1448, 842-1328, 852-1262, 856- |
| | 1553, 857-1320, 860-1610, 874- |
| | 1359, 876-1616, 889-1616, 893- |
| | 1527, 895-1616, 902-1615, 907- |
| | 1585, 907-1615, 916-1411, 921- |
| | 1418, 924-1616, 936-1616, 938- |
| | 1616, 946-1489, 948-1616, 952- |
| | 1616, 972-1614, 973-1614, 977- |
| | 1616, 980-1616, 981-1616, 985- |
| | 1496, 985-1616, 986-1616, 989- |
| | 1374, 990-1435, 1008-1616, 1009- |
| | 1514, 1009-1616, 1010-1616, |
| | 1014-1483, 1015-1616, 1041-1616, |
| | 1047-1616, 1050-1527, 1052-1589, |
| | 1053-1616, 1055-1616, 1056-1616, |
| | 1057-1616, 1075-1520, 1079-1616, |
| | 1095-1601, 1099-1616, 1100-1616, |
| | 1102-1616, 1108-1616, 1118-1616, |
| | 1124-1616, 1125-1607, 1133-1616, |
| | 1135-1616, 1144-1616, 1148-1616, |
| | 1151-1616, 1152-1616, 1159-1613, |
| | 1179-1616, 1217-1616, 1220-1616, |
| | 1226-1616, 1232-1616, 1244-1616, |
| | 1263-1616, 1326-1616, 1339-1616, |
| | 1367-1616, 1384-1616 |
| 39/5665403CB1/424 | 1-120, 1-160, 1-219, 4-330, 4- |
| | 424, 14-219 |
| 40/7493065CB1/1782 | 1-50, 1-303, 18-391, 19-548, 24- |
| | 306, 24-610, 26-763, 28-247, 29- |
| | 310, 33-711, 34-629, 34-832, 37- |
| | 263, 39-297, 41-289, 41-567, 86- |
| | 328, 86-385, 113-304, 176-454, |
| | 263-864, 275-858, 293-535, 569- |
| | 872, 634-1210, 837-1110, 837- |
| | 1328, 837-1355, 980-1121, 1077- |
| | 1300, 1077-1702, 1155-1771, |
| | 1155-1776, 1155-1782, 1199-1780 |
| 41/7493531CB1/846 | 1-265, 1-302, 1-575, 1-587, 144- |
| | 422, 145-418, 145-430, 145-757, |
| | 145-846, 149-7 19, 261-345 |
| 42/3321454CB1/3601 | 1-256, 6-256, 7-196, 8-198, 8- |
| | 256, 8-614, 8-694, 10-256, 10- |
| | 257, 13-198, 19-198, 20-256, 22- |
| | 256, 23-550, 23-572, 23-800, 29- |
| | 255, 44-681, 50-256, 54-256, |
| | 139-694, 412-921, 412-960, 480- |
| | 1154, 481-582, 481-1154, 798- |
| | 874, 799-1495, 801-969, 801-975, |
| | 801-1010, 801-1151, 801-1154, |
| | 941-1322, 1038-1461, 1050-1637, |
| | 1051-1866, 1407-1847, 1453-1729, |
| | 1453-1873, 1461-2220, 1469-1966, |
| | 1491-1621, 1604-1803, 1626-2086, |
| | 1741-2314, 1861-2104, 1861-2183, |
| | 1894-1980, 2106-2189, 2122-2233, |
| | 2174-2849, 2181-2283, 2182-2849, |
| | 2189-2812, 2214-2461, 2214-2747, |
| | 2286-2708, 2298-2961, 2303-2870, |
| | 2314-2398, 2340-2394, 2314-2392, |
| | 2385-2621, 2385-2646, 2440-2743, |
| | 2450-2708, 2519-3131, 2530-2784, |
| | 2530-3114, 2538-2742, 2610,2708, |
| | 2773-3049, 2961-3265, 2990-3048, |
| | 2997-3601, 3028-3247, 3049-3105, |
| | 3049-3109 |
| 43/189299CB1/2333 | 1-503, 91-260, 204-289, 204-608, |
| | 204-634, 204-652, 204-703, 204- |
| | 705, 204-771, 204-807, 204-824, |
| | 390-1119, 448-937, 449-1127, |
| | 514-1103, 514-1104, 572-1091, |
| | 573-1162, 581-1023, 581-1185, |
| | 581-1214, 581-1217, 597-1246, |
| | 598-1169, 615-815, 615-1150, |
| | 631-1093, 638-1310, 678-995, |
| | 691-1196, 702-1248, 706-1034, |
| | 712-991, 734-1000, 734-1123, |
| | 734-1328, 756-1345, 853-1404, |
| | 886-1507, 887-1572, 889-1573, |
| | 933-1523, 963-1573, 972-1478, |
| | 975-1547, 975-1670, 983-l677, |
| | 995-12O3, 1020-1486, 1022-1755, |
| | 1038-1493, 1047-1574, 1092-1699, |
| | 1114-1675, 1149-1594, 1150-1677, |
| | 1154-1905, 1174-1762, 1211-1829, |
| | 1213-1938, 1214-1451, 1214-1822, |
| | 1257,1835, 1259-1804, 1272-1996, |
| | 1273-1862, 1275-1733, 1277-1955, |
| | 1285-1825, 1285-1865, 1305-1842, |
| | 1305-1845, 1307-1616, 1307-1882, |
| | 1308-1938, 1311-1857, 1311-1859, |
| | 1312-1919, 1327-1857, 1332-1857, |
| | 1333-1852, 1335-1949, 1345-1804, |
| | 1348-1856, 1377-1801, 1385-1962, |
| | 1400-2091, 1402-2086, 1403-1683, |
| | 1403-1955, 1405-1862, 1405-1923, |
| | 1416-2017, 1417-1910, 1459-1940, |
| | 1464-1947, 1474-2076, 1481-2140, |
| | 1509-2065, 1529-2166, 1532-2156, |
| | 1535-2140, 1540-2097, 1556-1965, |
| | 1578-2209, 1585-1620, 1601-2211, |
| | 1611-2315, 1621-2318, 1623-2300, |
| | 1630-2305, 1662-2099, 1662-2319, |
| | 1759-2275, 1788-2241, 1790-2066, |
| | 1812-2245, 1834-2333, 1886-2236, |
| | 1907-2132 |
| 44/7488057CB1/2016 | 1-781, 42-2016, 152-787, 186- |
| | 706, 221-818, 233-762, 324-922, |
| | 358-864, 362-917, 476-1061, 524- |
| | 648, 526-648, 576-1144, 577- |
| | 1153, 583-1146, 587-1042, 616- |
| | 648, 616-649, 630-1087, 655- |
| | 1207, 661-1257, 668-1207, 668- |
| | 1240, 668-1241, 668-1262, 674- |
| | 1203, 674-1204, 681-1173, 695- |
| | 1283, 701-1283, 719-1220, 720- |
| | 749, 720-782, 720-792, 720-793, |
| | 763-1255, 763-1283, 764-1341, |
| | 786-1280, 791-1267, 791-1289, |
| | 791-1326, 791-1328, 791-1336, |
| | 791-1353, 791-1361, 791-1372, |
| | 803-927, 805-1283, 806-927, 806- |
| | 1028, 806-1066, 806-1072, 811- |
| | 1302, 816-1401, 845-1347, 855- |
| | 1478, 874-1514, 895-927, 921- |
| | 1468, 940-1541, 999-1064, 999- |
| | 1066, 999-1072, 1000-1060, 1004- |
| | 1072, 1042-1072, 1043-1072, |
| | 1340-2003 |
| 45/7486411CB1/1280 | 1-834, 6-834, 16-834, 102-834, |
| | 126-831, 128-834, 220-834, 223- |
| | 831, 736-1023, 811-1149, 813- |
| | 1023, 813-1149, 851-1273, 985- |
| | 1279, 1024-1149, 1051-1280, |
| | 1052-1280, 1055-1280 |
| 46/2005762CB1/709 | 1-278, 54-520, 54-528, 135-357, |
| | 155-679, 168-709, 181-345, 181- |
| | 685, 181-709, 239-699, 240-522, |
| | 276-476, 276-686, 276-709, 317- |
| | 505, 325-709, 327-699, 329-709, |
| | 337-578, 349-704, 459-671, 459- |
| | 696 |
| 47/2514091CB1/1699 | 1-539, 1-610, 1-656, 8-520, 12- |
| | 1699, 116-586, 225-974, 314- |
| | 1143, 320-546, 473-1044, 488- |
| | 1347, 595-1335, 630-1517, 677- |
| | 1207, 680-1392, 684-1436, 749- |
| | 1517, 767-1214, 818-1621, 828- |
| | 1376, 851-1664, 854-1363, 858- |
| | 1456, 883-1550, 975-1478, 979- |
| | 1522, 987-1653, 988-1458, 990- |
| | 1481, 1010-1576, 1052-1604, |
| | 1101-1699, 1199-1685 |
| 48/2726954CB1/3060 | 1-228, 1-242, 1-245, 1-261, 1- |
| | 263, 7-222, 7-567, 10-543, 11- |
| | 272, 22-279, 22-380, 37-306, 38- |
| | 303, 47-287, 62-327, 62-517, 85- |
| | 351, 87-346, 92-324, 92-339, 92- |
| | 351, 92-602, 105-326, 105-355, |
| | 105-372, 107-315, 108-348, 110- |
| | 257, 111-400, 114-426, 120-288, |
| | 120-341, 120-488, 139-299, 156- |
| | 419, 157-418, 158-494, 204-595, |
| | 234-367, 235-476, 261-436, 262- |
| | 352, 262-376, 262-737, 263-525, |
| | 267-534, 278-528, 299-700, 319- |
| | 589, 332-616, 345-788, 354-815, |
| | 359-841, 365-767, 385-515, 405- |
| | 636, 416-929, 419-642, 422-989, |
| | 423-673, 447-527, 462-723, 476- |
| | 759, 481-983, 482-755, 503-883, |
| | 504-801, 511-673, 525-708, 565- |
| | 800, 574-915, 595-1127, 602- |
| | 1151, 675-916, 679-1130, 686- |
| | 1122, 698-1132, 699-1143, 700- |
| | 1097, 712-1140, 723-1122, 736- |
| | 1136, 746-1046, 747-1122, 750- |
| | 1010, 750-1252, 751-1236, 760- |
| | 1160, 770-893, 780-1094, 795- |
| | 1048, 808-1412, 820-1027, 824- |
| | 1136, 826-1090, 837-1164, 840- |
| | 1142, 857-1125, 860-1291, 861- |
| | 1098, 861-1111, 875-1270, 913- |
| | 1274, 916-1277, 931-1376, 945- |
| | 1131, 948-1199, 951-1133, 994- |
| | 1322, 1004-1239, 1006-1398, |
| | 1011-1293, 1021-1137, 1026-1137, |
| | 1026-1144, 1037-1274, 1199-1583, |
| | 1210-1577, 1312-1611, 1329-1558, |
| | 1378-1608, 1515-2010, 1549-2129, |
| | 1903-2004, 1903-2209, 1903-2223, |
| | 1908-2339, 1914-2104, 1914-2160, |
| | 1925-2261, 1927-2130, 1938-2159, |
| | 1939-2244, 1961-2064, 1971-2243, |
| | 1974-2160, 1976-2238, 1985-2254, |
| | 1985-2392, 1986-2247, 1986-2460, |
| | 1986-2509, 1992-2484, 2038-2248, |
| | 2038-2274, 2041-2309, 2066-2290, |
| | 2089-2316, 2106-2409, 2126-2678, |
| | 2152-2682, 2182-2441, 2267-2544, |
| | 2267-2629, 2306-2595, 2307-2532, |
| | 2310-2717, 2312-2587, 2323-2541, |
| | 2342-2591, 2356-2675, 2359-2621, |
| | 2359-2759, 2365-2632, 2368-2616, |
| | 2371-2685, 2393-2919, 2396-2664, |
| | 2400-2675, 2411-2666, 2411-2779, |
| | 2413-2679, 2414-2690, 2430-2903, |
| | 2436-2742, 2436-2951, 2443-2997, |
| | 2469-2667, 2479-2709, 2500-2956, |
| | 2508-2759, 2515-3001, 2546-2909, |
| | 2547-3036, 2566-3036, 2572-3032, |
| | 2574-2994, 2582-3031, 2583-3040, |
| | 2609-3036, 2642-3036, 2646-3036, |
| | 2653-2891, 2653-2990, 2653-2994, |
| | 2666-2903, 2666-3051, 2671-2917, |
| | 2689-2939, 2690-2945, 2698-3037, |
| | 2707-3031, 2713-2854, 2715-2919, |
| | 2737-2939, 2737-2993, 2737-3036, |
| | 2739-2958, 2739-2975, 2739-3040, |
| | 2740-2995, 2750-3032, 2755-2944, |
| | 2757-3029, 2797-3060, 2801-3019, |
| | 2801-3033, 2801-3044, 2806-3037, |
| | 2808-3040, 2813-3034, 2821-3036, |
| | 2828-3046, 2832-3012, 2859-3036, |
| | 2866-3036, 2923-3036, 2934-3036 |
| 49/5406015CB1/746 | 1-253, 1-580, 1-746 |
| 50/2850658CB1/2303 | 1-36, 1-306, 1-458, 1-508, 2-171, |
| | 66-287, 88-502, 112-383, 119- |
| | 474, 126-279, 126-490, 126-502, |
| | 126-551, 176-491, 502-722, 612- |
| | 833, 684-888, 725-1284, 789- |
| | 1048, 1136-1488, 1141-1713, |
| | 1171-1321, 1220-1719, 1312-1581, |
| | 1357-1625, 1357-1829, 1515-1761, |
| | 1598-1788, 1630-1906, 1667-1815, |
| | 1699-1936, 1770-2303, 1775-2032, |
| | 1777-2260, 1795-2303, 1812-1956, |
| | 1812-2123, 1812-2154, 1812-2290, |
| | 1813-2102, 1825-1942 |
| 51/6579653CB1/604 | 1-120, 1-604 |
| 52/6819648CB1/1061 | 1-569, 1-652, 446-1061, 540-1061 |
| 53/2771521CB1/845 | 1-468, 47-823, 61-343, 61-356, |
| | 61-404, 61-414, 61-471, 61-477, |
| | 61-514, 61-538, 61-539, 61-540, |
| | 61-543, 61-544, 61-548, 61-549, |
| | 61-554, 61-582, 70-470, 77-629, |
| | 78-416, 78-574, 79-269, 80-520, |
| | 81-552, 81-578, 83-554, 89-378, |
| | 107-563, 111-510, 112-570, 113- |
| | 555, 130-711, 196-455, 263-821, |
| | 304-543, 332-841, 444-830, 495- |
| | 837, 532-830, 560-830, 570-830, |
| | 601-845, 623-830, 628-826, 634- |
| | 845, 637-831, 647-815, 672-804 |
| 54/7095792CB1/655 | 1-654, 1-655, 68-652, 87-546, |
| | 111-645 |
| 55/7112696CB1/1087 | 1-807, 6-611, 6-656, 6-693, 6- |
| | 695, 8-535, 29-398, 35-415, 51- |
| | 524, 243-1084, 278-1087 |
| 56/7759388CB1/2869 | 1-566, 1-625, 1-1039, 36-817, |
| | 59-623, 70-2869, 128-802, 323- |
| | 1043, 816-1345, 827-1381, 827- |
| | 1418, 869-1358, 1088-1721, 1257- |
| | 1568, 1399-2028, 1567-1827, |
| | 1567-1890, 1653-2230, 2140-2686, |
| | 2193-2730, 2245-2412, 2249-2869, |
| | 2312-2563 |
| 57/8165414CB1/2798 | 1-507, 54-559, 143-777, 288-746, |
| | 311-589, 312-706, 312-922, 324- |
| | 629, 344-618, 347-890, 350-1027, |
| | 351-832, 366-1065, 425-1037, |
| | 457-1246, 462-1065, 473-847, |
| | 481-1244, 498-1079, 504-1187, |
| | 506-964, 509-1032, 518-1113, |
| | 518-1174, 518,1245, 518-1379, |
| | 558-823, 567-842, 581-871, 631- |
| | 1124, 663-1316, 695-1434, 747- |
| | 1406, 748-1019, 767-1317, 822- |
| | 1369, 824-1133, 826-1486, 876- |
| | 1565, 884-1246, 930-1480, 943- |
| | 1634, 953-1608, 956-1402, 993- |
| | 1334, 1015-1686, 1036-1627, |
| | 1037-1721, 1039-1579, 1039-1634, |
| | 1065-1535, 1066,1614, 1067-1628, |
| | 1084-1576, 1118-1640, 1128-1651, |
| | 1132-1881, 1155-1783, 1165-1677, |
| | 1188-1461, 1212-1740, 1245-1977, |
| | 1246-1560, 1265-1523, 1265-1801, |
| | 1272-1558, 1276-1780, 1293-1546, |
| | 1299-1858, 1299-1968, 1300-1598, |
| | 1308-1816, 1309-2024, 1313-1595, |
| | 1315-1593, 1315-1595, 1315-1598, |
| | 1323-1595, 1323-1999, 1325-1595, |
| | 1326-1595, 1326-1609, 1328-1906, |
| | 1329-1594, 1329-1595, 1329-1596, |
| | 1329-1597, 1329-1598, 1329-1599, |
| | 1329-1603, 1329-1604, 1329-1910, |
| | 1329-1925, 1329-1926, 1330-1603, |
| | 1330-2008, 1331-1595, 1333-1595, |
| | 1338-1683, 1339-1599, 1342-1638, |
| | 1349-1968, 1352-1744, 1364-1619, |
| | 1376-1629, 1399-1862, 1404-2050, |
| | 1406-1699, 1408-1968, 1408-2102, |
| | 1415-1965, 1425-1713, 1430-1702, |
| | 1436,2036, 1448-1719, 1452-2125, |
| | 1459-1860, 1465-1971, 1476-1763, |
| | 1477-1996, 1478-2005, 1493-2083, |
| | 1493-2288, 1517-2104, 1520-2091, |
| | 1523-1779, 1546-2115, 1546-2303, |
| | 1552-1999, 1566-1846, 1567-1818, |
| | 1585-2090, 1585-2091, 1588-1834, |
| | 1588-1904, 1607-1877, 1615-2082, |
| | 1620-1884, 1632-1891, 1641-1903, |
| | 1644-2134, 1656-2482, 1674-2378, |
| | 1675-2408, 1680-1940, 1694-1949, |
| | 1694-1981, 1716-2087, 1727-2004, |
| | 1733-1979, 1735-2294, 1743-2212, |
| | 1744-2048, 1774-2042, 1774-2234, |
| | 1787-2080, 1790-2219, 1797-2082, |
| | 1803-2103, 1814-2523, 1826-2120, |
| | 1844-2605, 1858-2525, 1859-2291, |
| | 1863-2140, 1867-2745, 1868-2443, |
| | 1872-2120, 1879-2542, 1888-2158, |
| | 1890-2122, 1893-2539, 1903-2125, |
| | 1903-2157, 1903-2217, 1918-2195, |
| | 1921-2798, 1926-2535, 1937-2153, |
| | 1942-2212, 1942-2567, 1950-2545, |
| | 1950-2642, 1956-2195, 1987-2160, |
| | 2020-2195, 2020-2204 |
| 58/2540610CB1/3808 | 1-408, 1-600, 199-451, 287-854, |
| | 308-544, 315-561, 315-882, 330- |
| | 604, 393-871, 528-1047, 539- |
| | 1025, 550-1008, 553-1005, 559- |
| | 1029, 563-1025, 567-1006, 592- |
| | 1047, 602-827, 615-1047, 629- |
| | 1025, 641-944, 642-862, 646- |
| | 1094, 650-1046, 656-987, 658- |
| | 1046, 661-1047, 662-1025, 664- |
| | 1047, 667-1002, 667-1038, 671- |
| | 1025, 675-1025, 678-1047, 684- |
| | 994, 686-1025, 689-1025, 693- |
| | 1025, 693-1047, 696-957, 697- |
| | 931, 697-1047, 698-1033, 701- |
| | 1015, 702-1320, 721-1013, 726- |
| | 1047, 727-1046, 732-919, 747- |
| | 1047, 749-1012, 760-1029, 762- |
| | 1046, 769-1025, 769-1047, 771- |
| | 1047, 774-1023, 779-998, 779- |
| | 1020, 779-1046, 796-1021, 799- |
| | 829, 821-1029, 851-1047, 874- |
| | 1627, 898-1047, 965-1479, 1040- |
| | 1421, 1040-1441, 1040-1446, |
| | 1040-1491, 1040-1518, 1040-1519, |
| | 1040-1520, 1040-1524, 1040-1536, |
| | 1040-1537, 1040-1543, 1041-1411, |
| | 1041-1547, 1041-1583, 1046-1614, |
| | 1048-1285, 1048-1374, 1048-1514, |
| | 1048-1648, 1048-1656, 1048-1692, |
| | 1048-1694, 1048-1718, 1057-1537, |
| | 1058-1486, 1068-1472, 1071-1349, |
| | 1071-1476, 1071-1482, 1103-1451, |
| | 1124-1439, 1129-1445, 1135-1518, |
| | 1151-1389, 1151-1593, 1153-1668, |
| | 1155-1700, 1162-1751, 1169-1460, |
| | 1195-1985, 1197-1474, 1198-1895, |
| | 1225-1309, 1227-1398, 1231-1806, |
| | 1233-1713, 1259-1366, 1271-2001, |
| | 1273-1979, 1279-1523, 1281-1432, |
| | 1295-1872, 1296-1612, 1306-1969, |
| | 1323-1456, 1339-1614, 1353-1907, |
| | 1360-1909, 1361-1996, 1368-1528, |
| | 1383-2058, 1384-1907, 1396-1917, |
| | 1399-2019, 1415-2038, 1431-2164, |
| | 1439-1560, 1465-1728, 1477-2194, |
| | 1494-2204, 1507-1744, 1507-1912, |
| | 1519-1782, 1529-2183, 1552-2250, |
| | 1563-2182, 1571-2248, 1575-2093, |
| | 1578-2201, 1589-2151, 1594-1838, |
| | 1601-1772, 1601-2157, 1601-2167, |
| | 1601-2211, 1601-2284, 1601-2292, |
| | 1606-2086, 1627-1937, 1632-2047, |
| | 1644-1872, 1646-1897, 1649-2122, |
| | 1657-1950, 1666-2114, 1666-2284, |
| | 1676-2206, 1684-2317, 1689-1927, |
| | 1697-1915, 1712-2214, 1729-1926, |
| | 1734-2315, 1752-2279, 1755-1938, |
| | 1763-2313, 1766-2284, 1777-2291, |
| | 1780-2066, 1791-1988, 1803-2103, |
| | 1804-1837, 1823-2419, 1838-2129, |
| | 1838-2307, 1853-2125, 1859-2133, |
| | 1860-2404, 1874-2556, 1890-2157, |
| | 1890-2382, 1921-2225, 1947-2440, |
| | 1948-2442, 1973-2588, 1988-2246, |
| | 2004-2282, 2008-2585, 2022-2569, |
| | 2063-2177, 2082-2788, 2092-2205, |
| | 2099-2619, 2142-2711, 2161,2890, |
| | 2184-2826, 2194-2272, 2207-2772, |
| | 2213-2755, 2227-2794, 2227-2805, |
| | 2230-2650, 2238-2677, 2262-3035, |
| | 2264-2864, 2277-2814, 2278-2468, |
| | 2278-2710, 2278-2799, 2278-2849, |
| | 2283-2537, 2290-2564, 2292-2820, |
| | 2318-2586, 2319-2597, 2326-2591, |
| | 2331-2912, 2341-2928, 2343-2511, |
| | 2347-2456, 2351-2593, 2352-2605, |
| | 2368-3050, 2369-2930, 2389-2685, |
| | 2394-2634, 2401-2837, 2408-2953, |
| | 2409-2531, 2423-2996, 2431-2686, |
| | 2464-3093, 2477-3061, 2478-2700, |
| | 2485-2942, 2521-3052, 2534-3226, |
| | 2555-3112, 2564-2832, 2577-3161, |
| | 2578-3116, 2578-3266, 2584-3064, |
| | 2611-3110, 2619-2839, 2621-3264, |
| | 2622-3225, 2682-3207, 2685-3339, |
| | 2705-3189, 2707-3189, 2713-3195, |
| | 2732-3217, 2734-3350, 2737-3328, |
| | 2740-3238, 2746-3355, 2750-3090, |
| | 2750-3350, 2753-3404, 2756-3368, |
| | 2767-3428, 2770-3258, 2780-3328, |
| | 2781-3346, 2786-3415, 2788-3271, |
| | 2788-3272, 2794-2832, 2796-3363, |
| | 2803-3189, 2815-3739, 2818-3275, |
| | 2827-3317, 2838-3463, 2843-3523, |
| | 2846-3497, 2847-3586, 2848-3552, |
| | 2861-3383, 2862-3345, 2877-3281, |
| | 2878-3416, 2885-3342, 2890-3334, |
| | 2890-3344, 2904-3326, 2912-3559, |
| | 2913-3404, 2913-3453, 2913-3454, |
| | 2913-3462, 2918-3189, 2926-3365, |
| | 2934-3218, 2943-3617, 2966-3245, |
| | 2982-3193, 2986-3264, 2989-3580, |
| | 2992-3236, 2994-3776, 3002-3264, |
| | 3002-3267, 3002-3273, 3002-3298, |
| | 3004-3476, 3015-3292, 3016-3215, |
| | 3016-3287, 3016-3561, 3026-3749, |
| | 3027-3252, 3027-3306, 3028-3623, |
| | 3046-3703, 3054-3313, 3058-3287, |
| | 3058-3573, 3072-3592, 3074-3773, |
| | 3080-3297, 3081-3297, 3082-3334, |
| | 3087-3319, 3087-3331, 3088-3788, |
| | 3103-3697, 3108-3595, 3111-3671, |
| | 3121-3703, 3122-3705, 3124-3728, |
| | 3125-3759, 3131-3410, 3153-3284, |
| | 3153-3445, 3168-3777, 3183-3762, |
| | 3186-3759, 3186-3761, 3189-3756, |
| | 3196-3737, 3199-3771, 3203-3759, |
| | 3244-3283, 3259-3744, 3271-3787, |
| | 3274-3771, 3284-3765, 3290-3808, |
| | 3291-3783, 3297-3776, 3304-3769, |
| | 3306-3780, 3310-3772, 3310-3780, |
| | 3315-3765, 3318-3771, 3318-3774, |
| | 3318-3776, 3318-3783, 3319-3768, |
| | 3319-3782, 3320-3778, 3321-3778, |
| | 3324-3785, 3325-3774, 3334-3774, |
| | 3335-3773, 3338-3782, 3338-3785, |
| | 3346-3781, 3357-3619, 3384-3742, |
| | 3399-3679, 3400-3720, 3410-3678, |
| | 3410-3774, 3412-3776, 3412-3779, |
| | 3420-3575, 3428-3767, 3461-3740, |
| | 3467-3704, 3515-3808, 3580-3776 |
| 59/1593380CB1/1877 | 1-701, 31-712, 187-897, 189-663, |
| | 193-900, 229-704, 238-773, 239- |
| | 1107, 254-713, 265-686, 267-647, |
| | 268-602, 269-609, 270-613, 270- |
| | 666, 270-692, 271,640, 271-662, |
| | 271-693, 271-771, 273-929, 276- |
| | 648, 279708, 280-902, 294-669, |
| | 297-692, 297-720, 330-1059, 330- |
| | 1088, 341-1028, 343-912, 362- |
| | 1047, 396-1086, 405-619, 448- |
| | 1044, 459-1197, 465-1116, 470- |
| | 856, 521-970, 524-1080, 529-970, |
| | 536-1040, 574-1005, 586-1141, |
| | 626-842, 629-1078, 635-1084, |
| | 651-871, 658-1078, 728-1153, |
| | 792-999, 1034-1739, 1099-1631, |
| | 1135-1330, 1156-1830, 1167-1796, |
| | 1167-1877, 1168-1877, 1174-1664, |
| | 1227-1877 |
| 60/1480069CB1/1688 | 1-1255, 10-643, 122-683, 159- |
| | 256, 221-283, 272-698, 298-875, |
| | 417-1010, 431-817, 479-683, 640- |
| | 922, 743-1247, 981-1243, 1000- |
| | 1243, 1092-1398, 1092-1688 |
| 61/2310442CB1/776 | 1-448, 1-500, 1-533, 1-562, 1- |
| | 579, 1-602, 5-550, 5-646, 5-751, |
| | 10-534, 27-480, 27-776, 80-639 |
| 62/7503731CB1/3158 | 1-408, 1-600, 1-3123, 199-451, |
| | 287-854, 308-544, 315-561, 315- |
| | 882, 330-604, 393-871, 528-1047, |
| | 539-1025, 550-1008, 553-1005, |
| | 559-1029, 563-1025, 567-1006, |
| | 592-1047, 615-1047, 629-1025, |
| | 641-944, 642-862, 646-1094, 661- |
| | 1047, 662-1025, 664-1047, 667- |
| | 896, 667-1002, 667-1038, 671- |
| | 1025, 675-1025, 678-1047, 684- |
| | 994, 686-1025, 689-1025, 693- |
| | 1025, 693,1047, 696-957, 697- |
| | 1047, 698-1033, 701-1015, 702- |
| | 1320, 721-1013, 726-1047, 732- |
| | 919, 747-1047, 749-1012, 760- |
| | 1029, 769-1025, 769-1047, 771- |
| | 1047, 774-1023, 779-998, 779- |
| | 1020, 779-1046, 796-1021, 821- |
| | 1029, 851-1047, 874-1627, 898- |
| | 1047, 965-1479, 1040-1421, 1040- |
| | 1441, 1040-1446, 1040-1491, |
| | 1040-1518, 1040-1519, 1040-1520, |
| | 1040-1524, 1040-1536, 1040-1537, |
| | 1040-1543, 1041-1411, 1041-1421, |
| | 1041-1491, 1041-1547, 1048-1285, |
| | 1048-1514, 1048-1656, 1048-1692, |
| | 1048-1694, 1048-1718, 1057-1537, |
| | 1058-1486, 1071-1349, 1071-1476, |
| | 1071-1482, 1103-1451, 1129-1445, |
| | 1135-1518, 1151-1389, 1151-1593, |
| | 1153-1668, 1155-1700, 1162-1751, |
| | 1197-1474, 1233-1713, 1259-1366, |
| | 1279-1523, 1281-1432, 1296-1612, |
| | 1323-1456, 1339-1614, 1434-1489, |
| | 1465-1728, 1507-1744, 1567-1807, |
| | 1679-1961, 1689-1807, 1804-1980, |
| | 1804-1983, 1804-1996, 1804-2008, |
| | 1804-2015, 1804-2075, 1804-2078, |
| | 1804-2133, 1804-2166, 1804-2257, |
| | 1804-2376, 1805-1976, 1807-2408, |
| | 1809-2069, 1820-2405, 1823-2127, |
| | 1823-2277, 1823-2291, 1830-2110, |
| | 1849-2541, 1850-2002, 1850-2565, |
| | 1852-2073, 1866-2143, 1868-2123, |
| | 1868-2227, 1870-2427, 1877-2467, |
| | 1878-2115, 1878-2162, 1879-2147, |
| | 1884-2164, 1884-2581, 1892-2476, |
| | 1893-2144, 1893-2184, 1893-2581, |
| | 1895-2141, 1895-2350, 1899-2379, |
| | 1912-2187, 1913-2117, 1916-2194, |
| | 1916-2492, 1921-2211, 1922-2200, |
| | 1923-2167, 1925-2250, 1926-2394, |
| | 1926-2425, 1934-2154, 1936-2579, |
| | 1937-2540, 1940-2547, 1941-2204, |
| | 1944-2350, 1946-2214, 1949-2169, |
| | 1951-2214, 1951-2256, 1951-2507, |
| | 1952-2557, 1952-2598, 1959-2213, |
| | 1966-2636, 1971-2241, 1980-2168, |
| | 1986-2553, 1989-2257, 1995-2215, |
| | 1999-2237, 1999-2323, 2004-2359, |
| | 2007-2140, 2008-2259, 2008-2263, |
| | 2013-2359, 2020-2504, 2022-2504, |
| | 2028-2510, 2036-2264, 2045-2343, |
| | 2049-2665, 2052-2360, 2052-2643, |
| | 2061-2670, 2062-2291, 2062-2318, |
| | 2063-2289, 2065-2338, 2065-2405, |
| | 2065-2665, 2068-2719, 2071-2683, |
| | 2072-2334, 2074-2637, 2082-2743, |
| | 2085-2367, 2085-2573, 2087-2346, |
| | 2087-2348, 2088-2396, 2088-2397, |
| | 2095-2335, 2095-2643, 2101-2360, |
| | 2101-2730, 2103-2587, 2110-2355, |
| | 2111-2678, 2118-2504, 2130-2336, |
| | 2130-2359, 2130-2399, 2133-2590, |
| | 2139-2443, 2142-2395, 2142-2632, |
| | 2145-2402, 2145-2415, 2153-2778, |
| | 2158-2838, 2160-2791, 2161-2812, |
| | 2162-2901, 2167-2368, 2176-2698, |
| | 2177-2660, 2178-2446, 2186-2410, |
| | 2191-2416, 2192-2596, 2193-2440, |
| | 2193-2462, 2193-2731, 2194-2410, |
| | 2196-2676, 2197-2565, 2200-2657, |
| | 2205-2649, 2205-2659, 2219-2641, |
| | 2221-2462, 2221-2477, 2221-2703, |
| 62 | 2227-2874, 2228-2719, 2231-2500, |
| | 2232-2468, 2233-2504, 2241-2329, |
| | 2241-2680, 2242-2567, 2245-2501, |
| | 2249-2467, 2249-2501, 2249-2503, |
| | 2249-2533, 2254-2503, 2258-2932, |
| | 2259-2533, 2263-2676, 2265-2496, |
| | 2281-2560, 2284-2483, 2288-2498, |
| | 2288-2521, 2292-2496, 2297-2508, |
| | 2301-2579, 2304-2895, 2307-2551, |
| | 2307-2631, 2309-3091, 2314-2939, |
| | 2317-2579, 2317-2582, 2317-2588, |
| | 2317-2613, 2319-2791, 2330-2607, |
| | 2331-2602, 2331-2876, 2342-2567, |
| | 2342-2621, 2343-2938, 2359-2577, |
| | 2364-2566, 2369-2628, 2369-2706, |
| | 2369-3071, 2373-2602, 2373-2888, |
| | 2374-2814, 2374-2882, 2378-2633, |
| | 2382-2675, 2387-2907, 2395-2612, |
| | 2396-2612, 2396-2676, 2397-2649, |
| | 2402-2634, 2402-2646, 2402-2673, |
| | 2403-3103, 2412-2814, 2413-2622, |
| | 2418-3012, 2420-2716, 2421-2657, |
| | 2426-2610, 2426-2620, 2426-2629, |
| | 2426-2660, 2426-2986, 2436-3018, |
| | 2437-2720, 2437-3020, 2438-3091, |
| | 2440-3074, 2446-2725, 2449-2657, |
| | 2457-2749, 2460-2688, 2461-2710, |
| | 2465-3073, 2466-2746, 2468-2599, |
| | 2468-2760, 2468-2801, 2468-3073, |
| | 2471-2633, 2472-3068, 2473-3054, |
| | 2477-3001, 2482-2730, 2483-3092, |
| | 2494-2770, 2498-3077, 2501-3074, |
| | 2501-3130, 2503-2746, 2504-2574, |
| | 2504-3071, 2508-2786, 2508-2803, |
| | 2511-2762, 2511-3052, 2512-2868, |
| | 2514-3086, 2518-3074, 2521-2693, |
| | 2521-2820, 2522-2753, 2527-2813, |
| | 2530-2768, 2530-2776, 2542-2748, |
| | 2543-2830, 2546-2757, 2546-2813, |
| | 2546-2819, 2549-2934, 2558-2849, |
| | 2566-2750, 2566-2808, 2574-3059, |
| | 2578-2790, 2580-2844, 2586-2807, |
| | 2586-2828, 2588-2824, 2589-3086, |
| | 2590-2879, 2591-3102, 2593-2729, |
| | 2599-3080, 2600-2858, 2600-2884, |
| | 2606-3098, 2607-3100, 2609-2878, |
| | 2612-3091, 2617-3084, 2619-3084, |
| | 2621-3095, 2624-3091, 2625-3087, |
| | 2625-3095, 2629-3087, 2630-3080, |
| | 2633-3086, 2633-3089, 2633-3091, |
| | 2633-3098, 2634-2892, 2634-3083, |
| | 2634-3097, 2635-3093, 2636-3083, |
| | 2636-3093, 2636-3098, 2637-3080, |
| | 2637-3100, 2638-3080, 2639-3093, |
| | 2639-3094, 2639-3100, 2640-2936, |
| | 2640-3089, 2641-3100, 2642-3085, |
| | 2642-3091, 2645-3086, 2649-2927, |
| | 2649-2929, 2649-3089, 2650-2919, |
| | 2650-3088, 2651-3096, 2652-3085, |
| | 2653-3097, 2653-3100, 2656-3080, |
| | 2656-3099, 2658-3085, 2659-2908, |
| | 2659-3085, 2659-3091, 2659-3100, |
| | 2660-3084, 2661-3096, 2662-3086, |
| | 2664-3086, 2665-3100, 2667-3094, |
| | 2668-3091, 2670-3100, 2671-3122, |
| | 2672-2905, 2672-2913, 2672-2923, |
| | 2672-2926, 2672-3075, 2672-3086, |
| | 2673-2918, 2673-2922, 2673-2953, |
| | 2675-3015, 2675-3079, 2679-3085, |
| | 2679-3094, 2679-3100, 2682-2919, |
| | 2682-2939, 2683-3080, 2685-2907, |
| | 2685-3085, 2687-3093, 2690-3086, |
| | 2694-3091, 2694-3096, 2698-3086, |
| | 2699-3057, 2700-3041, 2700-3070, |
| | 2700-3090, 2701-2947, 2701-2989, |
| | 2702-3090, 2706-2941, 2711-2960, |
| | 2711-3158, 2714-2994, 2715-3035, |
| | 2716-3093, 2717-3091, 2718-3086, |
| | 2720-2959, 2724-3100, 2725-2993, |
| | 2725-3085, 2727-3091, 2727-3094, |
| | 2728-3091, 2730-3091, 2732-3086, |
| | 2735-2890, 2737-3080, 2740-2963, |
| | 2742-3084, 2743-3082, 2745-3091, |
| | 2752-2990, 2757-3053, 2758-3030, |
| | 2758-3091, 2760-3015, 2761-3091, |
| | 2762-2967, 2762-3091, 2765-3092, |
| | 2772-3093, 2775-3087, 2776-3055, |
| | 2776-3078, 2777-3090, 2778-3097, |
| | 2780-3091, 2782-3009, 2782-3019, |
| | 2799-3064, 2813-3091, 2816-3100, |
| | 2823-3077, 2823-3084, 2830-3146, |
| | 2831-3085, 2833-3098, 2849-3091, |
| | 2849-3100, 2850-3082, 2851-3082, |
| | 2852-3100, 2853-3093, 2855-3084, |
| | 2859-3090, 2861-3100, 2890-3069, |
| | 2906-3084, 2916-3086, 2918-3085, |
| | 2919-3091, 2920-3100, 2940-3072, |
| | 2941-3100, 2942-3091, 2949-3151, |
| | 2958-3084, 2964-3085, 2969-3086, |
| | 2979-3100, 2983-3084, 2983-3086, |
| | 2296-3090 |
| 63/7506368CB1/3024 | 1-154, 1-198, 1-271, 2-280, 18- |
| | 329, 18-3024, 24-239, 24-637, |
| | 25-613, 27-632, 27-699, 28-290, |
| | 39-296, 54-323, 55-322, 64-304, |
| | 79-347, 83-754, 84-253, 98-495, |
| | 103-620, 103-664, 103-747, 103- |
| | 803, 106-740, 109-342, 109-360, |
| | 109-386, 109-645, 115-183, 116- |
| | 598, 122-359, 122-379, 122-391, |
| | 124-385, 125-375, 127-408, 128- |
| | 417, 131-443, 137-505, 138-358, |
| | 156-340, 173-436, 173-671, 175- |
| | 434, 184-405, 221-612, 251-493, |
| | 251-824, 251-908, 252-721, 261- |
| | 549, 279-702, 279-718, 279-755, |
| | 279-784, 287-802, 288-552, 295- |
| | 546, 299-833, 326-461, 336-606, |
| | 349-633, 362-842, 375-858, 380- |
| | 842, 393-848, 393-851, 393-852, |
| | 422-653, 436-661, 439-693, 479- |
| | 743, 489-903, 492-901, 493-909, |
| | 495-776, 519-910, 528-690, 582- |
| | 817, 582-910, 756-1111, 787- |
| | 1370, 788-1111, 807-1111, 909- |
| | 1246, 909-1252, 909-1522, 923- |
| | 1111, 926-1177, 929-1111, 972- |
| | 1300, 982-1217, 989-1271, 999- |
| | 1118, 1015-1252, 1044-1528, |
| | 1127-1396, 1147-1558, 1177-1560, |
| | 1188-1677, 1291-1595, 1295-1960, |
| | 1356-1606, 1366-1908, 1427-2092, |
| | 1493-1992, 1527-2107, 1678-1715, |
| | 1678-1724, 1682-2284, 1714-1940, |
| | 1721-1982, 1769-2125, 1769-2305, |
| | 1774-1970, 1799-2416, 1812-2387, |
| | 1813-1859, 1832-2083, 1881-2201, |
| | 1892-2138, 1903-2347, 1905-2177, |
| | 1916-2227, 1916-2559, 1939-2068, |
| | 1949-2221, 1952-2138, 1953-2224, |
| | 1963-2232, 1963-2370, 1964-2225, |
| | 1964-2502, 1964-2556, 1970-2493, |
| | 2016-2253, 2016-2271, 2019-2287, |
| | 2020-2646, 2039-2624, 2043-2268, |
| | 2063-2679, 2067-2328, 2084-2386, |
| | 2104-2684, 2128-2660, 2151-2801, |
| | 2160-2419, 2244-2530, 2245-2642, |
| | 2284-2573, 2285-2511, 2288-2659, |
| | 2288-2695, 2301-2519, 2320-2568, |
| | 2337-2599, 2337-2737, 2342-2610, |
| | 2346-2594, 2349-2663, 2374-2642, |
| | 2378-2653, 2389-2644, 2389-2757, |
| | 2391-2657, 2392-2668, 2394-2992, |
| | 2400-2881, 2414-2722, 2421-3005, |
| | 2435-3014, 2447-2645, 2457-2687, |
| | 2457-3007, 2478-2934, 2485-2741, |
| | 2501-3024, 2517-3006, 2524-2887, |
| | 2544-3014, 2550-3010, 2560-3009, |
| | 2561-3018, 2587-3014, 2620-3014, |
| | 2624-3014, 2631-2869, 2631-3002, |
| | 2631-3024, 2644-2895, 2649-2895, |
| | 2667-2918, 2668-2923, 2685-3009, |
| | 2689-2832, 2693-2913, 2715-2917, |
| | 2715-2971, 2715-3014, 2717-2936, |
| | 2717-2953, 2717-3001, 2728-3010, |
| | 2733-2922, 2758-3024, 2779-3019, |
| | 2779-3023, 2786-3018, 2799-3024, |
| | 2806-3024, 2809-2989, 2837-3014, |
| | 2844-3024, 2867-3024, 2881-2945, |
| | 2912-3024 |
| 64/7509087CB1/948 | 1-948, 47-682, 50-107, 57-469, |
| | 61-267, 61-343, 61-356, 61-404, |
| | 61-405, 61-408, 61-414, 61-444, |
| | 70-452, 79-269, 79-437, 84-469, |
| | 89-402, 107-469, 110-444, 111- |
| | 197, 111-429, 111-437, 111-465, |
| | 111-469, 123-444, 126-348, 138- |
| | 469, 162-428, 179-934, 194-884, |
| | 196-455, 209-458, 243-363, 259- |
| | 544, 274-409, 283-948, 296-936, |
| | 317-469, 321-542, 468-901, 487- |
| | 732, 488-948, 489-724, 509-733, |
| | 523-786, 525-948, 532-937, 537- |
| | 939, 538-937, 539-933, 563-937, |
| | 568-837, 575-937, 591-849, 593- |
| | 924, 594-834, 598-948, 600-859, |
| | 600-865, 602-944, 605-935, 615- |
| | 899, 638-884, 638-898, 642-943, |
| | 643-722, 643-723, 643-730, 643- |
| | 753, 643-818, 643-899, 643-903, |
| | 644-666, 649-825, 651-878, 667- |
| | 937, 670-936, 677-937, 684-948, |
| | 686-937, 686-946, 687-947, 692- |
| | 938, 694-937, 705-933, 708-948, |
| | 719-936, 730-937, 735-933, 744- |
| | 938, 752-932, 762-897, 768-945, |
| | 776-946, 791-919 |

TABLE 5

| Polynucleotide | | |
|---|---|---|
| SEQ. | Incyte | Representative |
| ID NO: | Project ID: | Library |
| 33 | 2072638CB1 | THYMFET02 |
| 34 | 747515CB1 | ADRENOT03 |
| 35 | 7495641CB1 | BRAFNON02 |
| 36 | 2937340CB1 | BRAYDIN03 |
| 37 | 3765326CB1 | BRSTTUT01 |
| 38 | 948883CB1 | PANCNOT05 |
| 39 | 5665403CB1 | PITUNOT06 |
| 40 | 7493065CB1 | BRANDIN01 |
| 41 | 7493531CB1 | NOSEDIT02 |
| 42 | 3321454CB1 | DRGTNOT01 |
| 43 | 189299CB1 | HEARFET02 |
| 44 | 7488057CB1 | PLACFEF05 |
| 46 | 2005762CB1 | OVARNOT10 |
| 47 | 2514091CB1 | OVARDIN02 |
| 48 | 2726954CB1 | THYRNOT08 |
| 49 | 5406015CB1 | BRAMNOT01 |
| 50 | 2850658CB1 | BRSTNOT05 |
| 51 | 6579653CB1 | BRANDIT04 |
| 52 | 6819648CB1 | OVARDIR01 |
| 53 | 2771521CB1 | SPLNFET02 |
| 54 | 7095792CB1 | BRACDIR02 |
| 55 | 7112696CB1 | BRAENOK01 |
| 56 | 7759388CB1 | THYMNOE02 |
| 57 | 8165414CB1 | SINIDME01 |
| 58 | 2540610CB1 | BRACNOK02 |
| 59 | 1593380CB1 | BRAITUT13 |
| 60 | 1480069CB1 | CORPNOT02 |
| 61 | 2310442CB1 | PANCNOT08 |
| 62 | 7503731CB1 | MPHGNOT03 |
| 63 | 7506368CB1 | RATRNOT02 |
| 64 | 7509087CB1 | THYMTUT03 |

TABLE 6

| Library | Vector | Library Description |
|---|---|---|
| ADRENOT03 | PSPORT1 | Library was constructed using RNA isolated from the adrenal tissue |
| | | of a 17-year-old Caucasian male, who died from |
| | | cerebral anoxia. |
| BRACDIR02 | PCDNA2.1 | This random primed library was constructed using RNA isolated from |
| | | diseased corpus callosum tissue removed from a 57-year-old Caucasian male who died from |
| | | a cerebrovascular accident. Patient history included Huntington's disease and |
| | | emphysema. |
| BRACNOK02 | PSPORT1 | This amplified and normalized library was constructed using RNA |
| | | isolated from posterior cingulate tissue removed from an 85-year-old Caucasian female |
| | | who died from myocardial infarction and retroperitoneal hemorrhage. Pathology indicated |
| | | atherosclerosis, moderate to severe, involving the circle of Willis, middle cerebral, |
| | | basilar and vertebral arteries; infarction, remote, left dentate nucleus; and amyloid |
| | | plaque deposition consistent with age. There was mild to moderate |
| | | leptomeningeal fibrosis, especially over the convexity of the frontal lobe. There was mild |
| | | generalized atrophy involving all lobes. The white matter was mildly thinned. |
| | | Cortical thickness in the temporal lobes, both maximal and minimal, was |
| | | slightly reduced. The substantia nigra pars compacta appeared mildly depigmented. |
| | | Patient history included COPD, hypertension, and recurrent deep venous thrombosis. |
| | | 6.4 million independent clones from this amplified library were |
| | | normalized in one round using conditions adapted from Soares et al., |
| | | PNAS (1994) 91: 9228-9232 and Bonaldo et al., |
| | | Genome Research 6 (1996): 791. |
| BRAENOK01 | PSPORT1 | This amplified and normalized library was constructed using |
| | | RNA isolated from inferior parietal cortex tissue removed from a 35-year-old Caucasian |
| | | male who died from cardiac failure. Pathology indicated moderate leptomeningeal fibrosis |
| | | and multiple microinfarctions of the cerebral neocortex. There was evidence of shrunken |
| | | and slightly eosinophilic pyramidal neurons throughout the cerebral hemispheres. |
| | | There were multiple small microscopic areas of cavitation with surrounding gliosis |
| | | scattered throughout the cerebral cortex. Patient history included dilated |
| | | cardiomyopathy, congestive heart failure, and cardiomegaly. Patient medications |
| | | included simethicone, Lasix, Digoxin, Colace, Zantac, captopril, and |
| | | Vasotec. 1.08 million independent clones from this amplified library were normalized |
| | | in one round using conditions adapted from Soares et al., PNAS (1994) 91: 9228-9232 |
| | | and Bonaldo et al., Genome Research 6 (1996): 791, except that a |
| | | significantly longer (48 hours/round) reannealing hybridization was used. |
| BRAFNON02 | pINCY | This normalized frontal cortex tissue library was constructed from 10.6 million |
| | | independent clones from a frontal cortex tissue library. Starting RNA was made from |
| | | superior frontal cortex tissue removed from a 35-year-old Caucasian male who |
| | | died from cardiac failure. Pathology indicated moderate leptomeningeal fibrosis |
| | | and multiple microinfarctions of the cerebral neocortex. Grossly, the brain regions |
| | | examined and cranial nerves were unremarkable. No atherosclerosis of the |
| | | major vessels was noted. Microscopically, the cerebral hemisphere revealed |
| | | moderate fibrosis of the leptomeninges with focal calcifications. There was |
| | | evidence of shrunken and slightly eosinophilic pyramidal neurons throughout |
| | | the cerebral hemispheres. There were also multiple small microscopic areas |
| | | of cavitation with surrounding gliosis scattered throughout the cerebral cortex. |
| | | Patient history included dilated cardiomyopathy, congestive heart failure, |
| | | cardiomegaly, and an enlarged spleen and liver. Patient medications included |
| | | simethicone, Lasix, Digoxin, Colace, Zantac, captopril, and |
| | | Vasotec. The library was normalized in two rounds using conditions adapted |
| | | from Soares et al., PNAS (1994) 91: 9228 and Bonaldo et al., Genome Research |
| | | (1996) 6: 791, except that a significantly longer (48 hours/round) reannealing |
| | | hybridization was used. |
| BRAITUT13 | pINCY | Library was constructed using RNA isolated from brain tumor tissue removed |
| | | from the left frontal lobe of a 68-year-old Caucasian male during excision of a |
| | | cerebral meningeal lesion. Pathology indicated a meningioma in the left frontal lobe. |
| BRAMNOT01 | pINCY | Library was constructed using RNA isolated from medulla tissue removed |
| | | from the brain of a 35-year-old Caucasian male who died from cardiac failure. |
| | | Pathology indicated moderate leptomeningeal fibrosis and multiple microinfarctions of the |
| | | cerebral neocortex. Microscopically, the cerebral hemisphere revealed moderate fibrosis |
| | | of the leptomeninges with focal calcifications. There was evidence of shrunken and slightly |
| | | eosinophilic pyramidal neurons throughout the cerebral hemispheres. In addition, scattered |
| | | throughout the cerebral cortex, there were multiple small microscopic areas of |
| | | cavitation with surrounding gliosis. Patient history included dilated cardiomyopathy, congestive |
| | | heart failure, cardiomegaly and an enlarged spleen and liver. |
| BRANDIN01 | pINCY | This normalized pineal gland tissue library was constructed from. 4 million independent |
| | | clones from a pineal gland tissue library from two different donors. Starting RNA was |
| | | made from pooled pineal gland tissue removed from two Caucasian |
| | | females: a 68-year-old (donor A) who died from congestive heart failure and a 79-year-old |
| | | (donor B) who died from pneumonia. Neuropathology for donor A indicated mild to moderate |
| | | Alzheimer disease, atherosclerosis, and multiple infarctions. Neuropathology for donor |
| | | B indicated severe Alzheimer disease, arteriolosclerosis, cerebral amyloid |
| | | angiopathy and multiple infarctions. There were diffuse and neuritic amyloid plaques |
| | | and neurofibrillary tangles throughout the brain sections examined in both donors. |
| | | Patient history included diabetes mellitus, rheumatoid arthritis, |
| | | hyperthyroidism, amyloid heart disease, and dementia in donor A; and pseudophakia, |
| | | gastritis with bleeding, glaucoma, peropheral vascular disease, COPD, delayed onset |
| | | tonic/clonic seizures, and transient ischemic attack in donor B. The |
| | | library was normalized in one round using conditions adapted from Soares |
| | | et al., PNAS (1994) 91: 9228-9232 and Bonaldo |
| | | et al., Genome Research 6 (1996): 791, except that a significantly longer |
| | | (48 hours/round) reannealing hybridization was used. |
| BRANDIT04 | pINCY | Library was constructed using RNA isolated from pineal gland tissue removed from |
| | | a 68-year-old Caucasian female who died from congestive heart failure. Neuropathology |
| | | indicated mild to moderate Alzheimer disease, atherosclerosis, and |
| | | multiple infarctions. Microscopically, there were diffuse and neuritic amyloid |
| | | plaques throughout the cerebral cortex. There were neurofibrillary tangles in the |
| | | temporal lobes particularly the entorhinal cortex. The frontal cortex contained |
| | | scattered, ballooned neurons. The amygdala contained marked gliosis, neuritic |
| | | plaques and intracellular neurofibrillary tangles. The hippocampus contained |
| | | neuritic and diffuse plaques, and neurofibrillaiy tangles. The thalamus contained |
| | | diffuse and focal neuritic amyloid plaques and scattered neurofibrillary tangles. |
| | | There was area of cystic cavitation with surrounding gliosis in the left globus pallidus. |
| | | The pallidum contained scattered intracellular neurofibrillary tangles. The |
| | | caudate, putamen and nucleus accumbens contained diffuse plaques. There was |
| | | an area of cystic cavitation with lipid-laden macrophages in the right cerebellar |
| | | hemisphere. Patient history included diabetes mellitus, rheumatoid arthritis, |
| | | hyperthyroidism, amyloid heart disease, and dementia. |
| BRAYDIN03 | pINCY | This normalized library was constructed from 6.7 million independent clones |
| | | from a brain tissue library. Starting RNA was made from RNA isolated from diseased |
| | | hypothalamus tissue removed from a 57-year-old Caucasian male who died from a |
| | | cerebrovascular accident. Patient history included Huntington's disease and |
| | | emphysema. The library was normalized in 2 rounds using conditions adapted |
| | | from Soares et al., PNAS (1994) 91: 9228 and Bonaldo et al., Genome Research (1996) |
| | | 6: 791, except that a significantly longer (48-hours/round) reannealing hybridization |
| | | was used. The library was linearized and recircularized to select |
| | | for insert containing clones. |
| BRSTNOT05 | PSPORT1 | Library was constructed using RNA isolated from breast tissue removed |
| | | from a 58-year-old Caucasian female during a unilateral extended simple mastectomy. |
| | | Pathology for the associated tumor tissue indicated multicentric invasive grade 4 |
| | | lobular carcinoma. Patient history included skin cancer, rheumatic heart disease, |
| | | osteoarthritis, and tuberculosis. Family history included cerebrovascular and |
| | | cardiovascular disease, breast and prostate cancer, and type I diabetes. |
| BRSTTUT01 | PSPORT1 | Library was constructed using RNA isolated from breast tumor tissue |
| | | removed from a 55-year-old Caucasian female during a unilateral extended simple mastectomy. |
| | | Pathology indicated invasive grade 4 mammary adenocarcinoma of mixed lobular |
| | | and ductal type, extensively involving the left breast. The tumor was identified in the deep |
| | | dermis near the lactiferous ducts with extracapsular extension. Seven mid and low and five |
| | | high axillary lymph nodes were positive for tumor. Proliferative fibrocysytic changes were |
| | | characterized by apocrine metaplasia, sclerosing adenosis, cyst formation, and ductal |
| | | hyperplasia without atypia. Patient history included atrial tachycardia, blood in the stool, |
| | | and a benign breast neoplasm. Family history included benign hypertension, |
| | | atherosclerotic coronary artery disease, cerebrovascular disease, |
| | | and depressive disorder. |
| CORPNOT02 | pINCY | Library was constructed using RNA isolated from diseased corpus |
| | | callosum tissue removed from the brain of a 74-year-old |
| | | Caucasian male who died from Alzheimer's disease. |
| DRGTNOT01 | pINCY | Library was constructed using RNA isolated from dorsal root ganglion tissue |
| | | removed from the thoracic spine of a 32-year-old Caucasian male who died from |
| | | acute pulmonary edema and bronchopneumonia, bilateral pleural and pericardial |
| | | effusions, and malignant lymphoma (natural killer cell type). Patient history included |
| | | probable cytomegalovirus infection, hepatic congestion and steatosis, splenomegaly, |
| | | hemorrhagic cystitis, thyroid hemorrhage, and Bell's palsy. Surgeries |
| | | included colonoscopy, large intestine biopsy, adenotonsillectomy, and nasopharyngeal |
| | | endoscopy and biopsy; treatment included radiation therapy. |
| HEARFET02 | pINCY | Library was constructed using RNA isolated from heart tissue removed |
| | | from a Caucasian male fetus, who was stillborn |
| | | with a hypoplastic left heart and died at 23 weeks' gestation. |
| MPHGNOT03 | PBLUESCRIPT | Library was constructed using RNA isolated from plastic adherent |
| | | mononuclear cells isolated from buffy coat units |
| | | obtained from unrelated male and female donors. |
| NOSEDIT02 | pINCY | Library was constructed using RNA isolated from nasal polyp tissue. |
| OVARDIN02 | pINCY | This normalized ovarian tissue library was constructed from 5.76 million |
| | | independent clones from an ovary library. Starting RNA was made from diseased |
| | | ovarian tissue removed from a 39-year-old Caucasian female during total abdominal |
| | | hysterectomy, bilateral salpingo-oophorectomy, dilation andcurettage, partial colectomy, |
| | | incidental appendectomy, and temporary colostomy. Pathology indicated the right and |
| | | left adnexa, mesentery and muscularis propria of the sigmoid |
| | | colon were extensively involved by endometriosis. Endometriosis also involved |
| | | the anterior and posterior serosal surfaces of the uterus and the cul-de-sac. |
| | | The endometrium was proliferative. Pathology for the associated tumor tissue indicated |
| | | multiple (3 intramural, 1 subserosal) leiomyomata. The patient presented with abdominal |
| | | pain and infertility. Patient history included scoliosis. Family history included hyperlipidemia, |
| | | benign hypertension, atherosclerotic coronary artery disease, depressive disorder, |
| | | brain cancer, and type II diabetes. The library was normalized in two rounds using conditions |
| | | adapted from Soares et al., PNAS(1994) 91: 9228 and Bonaldo et al., Genome |
| | | Research 6 (1996): 791, except that a significantly longer (48-hours/round) |
| | | reannealing hybridization was used. |
| OVARDIR01 | PCDNA2.1 | This random primed library was constructed using RNA isolated from |
| | | right ovary tissue removed from a 45-year-old Caucasian female during total abdominal |
| | | hysterectomy, bilateral salpingo-oophorectomy, vaginal suspension and fixation, |
| | | and incidental appendectomy. Pathology indicated stromal hyperthecosis of the right |
| | | and left ovaries. Pathology for the matched tumor tissue indicated a dermoid cyst |
| | | (benign cystic teratoma) in the left ovary. Multiple (3) intramural |
| | | leiomyomata were identified. The cervix showed squamous metaplasia. Patient |
| | | history included metrorrhagia, female stress incontinence, alopecia, depressive disorder, |
| | | pneumonia, normal delivery, and deficiency anemia. Family history included |
| | | benign hypertension, atherosclerotic coronary artery disease, hyperlipidemia, and |
| | | primary tuberculous complex. |
| OVARNOT10 | pINCY | Library was constructed using RNA isolated from left ovarian tissue |
| | | removed from a 52-year-old Caucasian female during a total abdominal hysterectomy, |
| | | incidental appendectomy, and bilateral salpingo-oophorectomy. Pathology indicated a |
| | | paratubal cyst in the left fallopian tube and a mesothelial-lined peritoneal cyst. Pathology |
| | | for the associated tumor tissue indicated multiple (9 intramural, 4 subserosal) |
| | | leiomyomata. Patient history included hyperlipidemia. Family history |
| | | included myocardial infarction, type II diabetes, atherosclerotic coronary artery |
| | | disease, hyperlipidemia, and cerebrovascular disease. |
| PANCNOT05 | PSPORT1 | Library was constructed using RNA isolated from the pancreatic |
| | | tissue of a 2-year-old Hispanic male who died from |
| | | cerebral anoxia. |
| PANCNOT08 | pINCY | Library was constructed using RNA isolated from pancreatic tissue |
| | | removed from a 65-year-old Caucasian female during radical subtotal pancreatectomy. |
| | | Pathology for the associated tumor tissue indicated an invasive grade 2 adenocarcinoma. |
| | | Patient history included type II diabetes, osteoarthritis, cardiovascular disease, benign |
| | | neoplasm in the large bowel, and a cataract. Previous surgeries included a total |
| | | splenectomy, cholecystectomy, and abdominal hysterectomy. Family history |
| | | included cardiovascular disease, type II diabetes, and stomach cancer. |
| PITUNOT06 | pINCY | Library was constructed using RNA isolated from pituitary gland |
| | | tissue removed from a 55-year-old male who died from |
| | | chronic obstructive pulmonary disease. Neuropathology indicated there were no gross |
| | | abnormalities, other than mild ventricular enlargement. There was no apparent |
| | | microscopic abnormality in any of the neocortical areas examined, except |
| | | for a number of silver positive neurons with apical dendrite staining, particularly |
| | | in the frontal lobe. The significance of this was undetermined. The only other |
| | | microscopic abnormality was that there was prominent silver staining with some swollen |
| | | axons in the CA3 region of the anterior and posterior hippocampus. Microscopic |
| | | sections of the cerebellum revealed mild Bergmann's gliosis in the Purkinje |
| | | cell layer. Patient history included schizophrenia. |
| PLACFEF05 | PCMV-ICIS | Library was constructed using RNA isolated from placental tissue |
| | | removed from a Caucasian fetus, who died after 16 weeks' gestation from |
| | | wrapped around the head (3 times) and the shoulders (1 time). Serology was |
| | | positive for anti-CMV and remaining serologies were negative. Family |
| | | history included multiple pregnancies and live births, and an abortion in the mother. |
| RATRNOT02 | PSPORT1 | Library was constructed using RNA isolated from the right |
| | | atrium tissue of a 39-year-old Caucasian male, who died from a |
| | | gunshot wound. |
| SINIDME01 | PCDNA2.1 | This 5′ biased random primed library was constructed |
| | | using RNA isolated from diseased ileum tissue removed from a 29- |
| | | year-old Caucasian female during jejunostomy. Pathology indicated mild chronic |
| | | inflammation. The patient presented with ulcerative colitis. Patient history included |
| | | a benign neoplasm of the large bowel. Patient medications included Asacol, |
| | | Rowasa, Clomid and Pergonol. Family history included benign hypertension in |
| | | the mother, and colon cancer and cerebrovascular accident in the grandparent(s). |
| SPLNFET02 | pINCY | Library was constructed using RNA isolated from spleen tissue |
| | | removed from a Caucasian male fetus, who died at 23 weeks' gestation. |
| THYMFET02 | pINCY | Library was constructed using RNA isolated from thymus |
| | | tissue removed from a Caucasian female fetus, who died at 17 |
| | | weeks' gestation from anencephalus. |
| THYMNOE02 | PCDNA2.1 | This 5′ biased random primed library was |
| | | constructed using RNA isolated from thymus tissue removed from a 3-year-old |
| | | Hispanic male during a thymectomy and closure of a patent ductus arteriosus. |
| | | The patient presented with severe pulmonary stenosis and cyanosis. |
| | | Patient history included a cardiac catheterization and echocardiogram. |
| | | Previous surgeries included Blalock-Taussig shunt and pulmonary valvotomy. |
| | | The patient was not taking any medications. Family history included |
| | | benign hypertension, osteoarthritis, depressive disorder, and extrinsic |
| | | asthma in the grandparent(s). |
| THYMTUT03 | pINCY | Library was constructed using RNA isolated from |
| | | thymus tumor tissue removed from a 56-year-old Caucasian female |
| | | during total thymectomy. Pathology indicated the neoplastic cells were |
| | | negative for all markers (AEAl/AE3, CAll 5.2, wide spectrum keratin, |
| | | LCA, Ber-H2, L26, CD3, CD31, S-10O, actin, and desmin). |
| | | Ultrastructurally, the neoplastic cells showed epithelial differentiation, |
| | | such as desmosomes, tonofilaments that support the diagnosis of |
| | | malignant thymoma. The patient presented with persistent thymus |
| | | hyperplasia and deficiency anemia. Patient history included cardiac |
| | | dysrhythmia, left bundle branch block and normal delivery. Patient |
| | | medications included Allopurinol. |
| THYRNOT08 | pINCY | Library was constructed using RNA isolated from the |
| | | diseased left thyroid tissue removed from a 13-year-old Caucasian |
| | | female during a complete thyroidectomy. Pathology indicated lymphocytic |
| | | thyroiditis. Pathology for the matched tumor tissue indicated grade I papillary |
| | | carcinoma. Multiple lymph nodes from the right, left, and midline section |
| | | of the neck were negative for tumor. Fragments of the thymus were benign. |
| | | Fibroadipose tissue was identified in the right inferior and superior parathyroid |
| | | regions. Multiple lymph nodes (2 of 6) from the right side of the neck |
| | | contained microscopic foci of metastatic papillary carcinoma. Patient |
| | | history included attention deficit disorder with hyperactivity. Previous surgeries |
| | | included an operative procedure on the external ear. Patient medications |
| | | included Prozac. Family history included chronic obstructive asthma in the |
| | | mother; alcohol abuse, benign hypertension, and depressive disorder |
| | | in the grandparent(s); and attention deficit disorder with hyperactivity in the sibling(s). |

TABLE 7

| Program | Description | Reference | Parameter Threshold |
|---|---|---|---|
| ABI | A program that removes vector sequences | Applied Biosystems, Foster City, | |
| FACTURA | and masks ambiguous bases in nucleic acid | CA. | |
| | sequences. | | |
| ABI/PARACEL | A Fast Data Finder useful in comparing and | Applied Biosystems, Foster City, | Mismatch < 50% |
| FDF | annotating amino acid or nucleic acid | CA; Paracel Inc., Pasadena, CA. | |
| | sequences. | | |
| ABI Auto | A program that assembles nucleic acid | Applied Biosystems, Foster City, | |
| Assembler | sequences. | CA. | |
| BLAST | A Basic Local Alignment Search Tool useful | Altschul, S. F. et al. (1990) J. Mol. | ESTs: Probability value = 1.0E−8 |
| | in sequence similarity search for amino acid | Biol. 215: 403-410; Altschul, S. F. et | or less; Full Length |
| | and nucleic acid sequences. BLAST includes | al. (1997) Nucleic Acids Res. | sequences: Probability value = 1.0E−10 |
| | five functions: blastp, blastn, blastx, tblastn, | 25: 3389-3402. | or less |
| | and tblastx. | | |
| FASTA | A Pearson and Lipman algorithm that searches | Pearson, W. R. and D. J. Lipman | ESTs: fasta E value = 1.06E−6; |
| | for similarity between a query sequence and a | (1988) Proc. Natl. Acad Sci. USA | Assembled ESTs: fasta |
| | group of sequences of the same type. FASTA | 85: 2444-2448; Pearson, W. R. | Identity = 95% or greater and |
| | comprises as least five functions: fasta, tfasta, | (1990) Methods Enzymol. 183: 63-98; | Match length = 200 bases or |
| | fastx, tfastx, and ssearch. | and Smith, T. F. and M. S. Waterman | greater; fastx E value = 1.0E−8 |
| | | (1981) Adv. Appl. Math. | or less; Full Length sequences: |
| | | 2: 482-489. | fastx score = 100 or greater |
| BLIMPS | A BLocks IMProved Searcher that matches a | Henikoff, S. and J. G. Henikoff | Probability value = 1.0E−3 or |
| | sequence against those in BLOCKS, PRINTS, | (1991) Nucleic Acids Res. 19: 6565-6572; | less |
| | DOMO, PRODOM, and PFAM databases to | Henikoff, J. G. and S. | |
| | search for gene families, sequence homology, | Henikoff (1996) Methods Enzymol. | |
| | and structural fingerprint regions. | 266: 88-105; and Attwood, T. K. et | |
| | | al. (1997) J. Chem. Inf. Comput. | |
| | | Sci. 37: 417-424. | |
| HMMER | An algorithm for searching a query sequence | Krogh, A. et al. (1994) J. Mol. Biol. | PFAM hits: Probability value = 1.0E−3 |
| | against hidden Markov model (HMM)-based | 235: 1501-1531; Sonnhammer, E. L. L. | or less; Signal peptide |
| | databases of protein family consensus | et al. (1988) Nucleic Acids | hits: Score = 0 or greater |
| | sequences, such as PFAM. | Res. 26: 320-322; Durbin, R. et al. | |
| | | (1998) Our World View, in a | |
| | | Nutshell, Cambridge Univ. Press, | |
| | | pp. 1-350. | |
| ProfileScan | An algorithm that searches for structural and | Gribskov, M. et al. (1988) CABIOS | Normalized quality |
| | sequence motifs in protein sequences that | 4: 61-66; Gribskov, M. et al. (1989) | score ≧ GCG-specified “HIGH” |
| | match sequence patterns defined in Prosite. | Methods Enzymol. 183: 146-159; | value for that particular |
| | | Bairoch, A. et al. (1997) Nucleic | Prosite motif. Generally, |
| | | Acids Res. 25: 217-221. | score = 1.4-2.1. |
| Phred | A base-calling algorithm that examines | Ewing, B. et al. (1998) Genome | |
| | automated sequencer traces with high | Res. 8: 175-185; Ewing, B. and P. | |
| | sensitivity and probability. | Green (1998) Genome Res. 8: 186-194. | |
| Phrap | A Phils Revised Assembly Program including | Smith, T. F. and M. S. Waterman | Score = 120 or greater; Match |
| | SWAT and CrossMatch, programs based on | (1981) Adv. Appl. Math. 2: 482-489; | length = 56 or greater |
| | efficient implementation of the Smith- | Smith, T. F. and M. S. Waterman | |
| | Waterman algorithm, useful in searching | (1981) J. Mol. Biol. | |
| | sequence homology and assembling DNA | 147: 195-197; and Green, P., | |
| | sequences. | University of Washington, Seattle, | |
| | | WA. | |
| Consed | A graphical tool for viewing and editing Phrap | Gordon, D. et al. (1998) Genome | |
| | assemblies. | Res. 8: 195-202. | |
| SPScan | A weight matrix analysis program that scans | Nielson, H. et al. (1997) Protein | Score = 3.5 or greater |
| | protein sequences for the presence of | Engineering 10: 1-6; Claverie, J. M. | |
| | secretory signal peptides. | and S. Audic (1997) CABIOS | |
| | | 12: 431-439. | |
| TMAP | A program that uses weight matrices to | Persson, B. and P. Argos (1994) J. | |
| | delineate transmembrane segments on protein | Mol. Biol. 237: 182-192; Persson, B. | |
| | sequences and determine orientation. | and P. Argos (1996) Protein Sci. | |
| | | 5: 363-371. | |
| TMHMMER | A program that uses a hidden Markov model | Sonnhammer, E. L. et al. (1998) | |
| | (HMM) to delineate transmembrane segments | Proc. Sixth Intl. Conf. On | |
| | on protein sequences and determine | Intelligent Systems for Mol. Biol., | |
| | orientation. | Glasgow et al., eds., The Am. | |
| | | Assoc. for Artificial Intelligence | |
| | | (AAAI) Press, Menlo Park, CA, and | |
| | | MIT Press, Cambridge, MA, pp. | |
| | | 175-182. | |
| Motifs | A program that searches amino acid sequences | Bairoch, A. et al. (1997) Nucleic | |
| | for patterns that matched those defined in | Acids Res. 25: 217-221; Wisconsin | |
| | Prosite. | Package Program Manual, version | |
| | | 9, page M51-59, Genetics Computer | |
| | | Group, Madison, WI. | |

TABLE 8

| | | | | | | | | | | Cauc- | Afri- | | His- |
|---|---|---|---|---|---|---|---|---|---|---|---|---|---|
| | | | | | | | | | | asian | can | Asian | panic |
| | | | | | | | | | | Allele | Allele | Allele | Allele |
| SEQ | | | | | | EST | All- | All- | | 1 | 1 | 1 | 1 |
| ID | | | | EST | CB1 | All- | ele | ele | Amino | fre- | fre- | fre- | fre- |
| NO: | PID | EST ID | SNP ID | SNP | SNP | ele | 1 | 2 | Acid | quency | quency | quency | quency |
| 63 | 7506368 | 1262740H1 | SNP00007141 | 148 | 2110 | G | C | G | noncoding | 0.41 | n/a | n/a | n/a |
| 63 | 7506368 | 1288420H1 | SNP00032285 | 44 | 2674 | T | T | C | noncoding | n/a | n/a | n/a | n/a |
| 63 | 7506368 | 1678140H1 | SNP00007141 | 95 | 2110 | C | C | G | noncoding | 0.41 | n/a | n/a | n/a |
| 63 | 7506368 | 1889769H1 | SNP00055076 | 232 | 2609 | A | A | C | noncoding | n/d | n/a | n/a | n/a |
| 63 | 7506368 | 1963250H1 | SNP00007141 | 146 | 2110 | G | C | G | noncoding | 0.41 | n/a | n/a | n/a |
| 63 | 7506368 | 1973196H1 | SNP00007115 | 134 | 306 | A | C | A | K52 | 0.40 | n/a | n/a | n/a |
| 63 | 7506368 | 2197283H1 | SNP00007115 | 185 | 306 | A | C | A | K52 | 0.40 | n/a | n/a | n/a |
| 63 | 7506368 | 2447843H1 | SNP00055076 | 153 | 2609 | A | A | C | noncoding | n/d | n/a | n/a | n/a |
| 63 | 7506368 | 2518388H1 | SNP00007114 | 43 | 106 | G | T | G | noncoding | 0.21 | n/a | n/a | n/a |
| 63 | 7506368 | 2733260H1 | SNP00007116 | 79 | 660 | T | T | C | C170 | 0.50 | n/a | n/a | n/a |
| 63 | 7506368 | 2782753H1 | SNP00007116 | 177 | 660 | C | T | C | C170 | 0.50 | n/a | n/a | n/a |
| 63 | 7506368 | 2817767H1 | SNP00007141 | 25 | 2110 | C | C | G | noncoding | 0.41 | n/a | n/a | n/a |
| 63 | 7506368 | 2872534H1 | SNP00007141 | 155 | 2110 | C | C | G | noncoding | 0.41 | n/a | n/a | n/a |
| 63 | 7506368 | 2905668H1 | SNP00007115 | 206 | 306 | C | C | A | N52 | 0.40 | n/a | n/a | n/a |
| 63 | 7506368 | 2938935H1 | SNP00055076 | 236 | 2609 | A | A | C | noncoding | n/d | n/a | n/a | n/a |
| 63 | 7506368 | 3181915H1 | SNP00007114 | 89 | 87 | T | T | G | noncoding | 0.21 | n/a | n/a | n/a |
| 63 | 7506368 | 3219591H1 | SNP00007116 | 70 | 660 | C | T | C | C170 | 0.50 | n/a | n/a | n/a |
| 63 | 7506368 | 3356882H1 | SNP00007114 | 25 | 106 | G | T | G | noncoding | 0.21 | n/a | n/a | n/a |
| 63 | 7506368 | 3356882H1 | SNP00007115 | 226 | 306 | A | C | A | K52 | 0.40 | n/a | n/a | n/a |
| 63 | 7506368 | 3441234H1 | SNP00007142 | 32 | 2680 | C | C | T | noncoding | n/a | n/a | n/a | n/a |
| 63 | 7506368 | 3514705H1 | SNP00007115 | 180 | 306 | C | C | A | N52 | 0.40 | n/a | n/a | n/a |
| 63 | 7506368 | 3630850H1 | SNP00007116 | 220 | 660 | T | T | C | C170 | 0.50 | n/a | n/a | n/a |
| 63 | 7506368 | 3725180H1 | SNP00007115 | 27 | 306 | A | C | A | K52 | 0.40 | n/a | n/a | n/a |
| 63 | 7506368 | 3787129H1 | SNP00007142 | 267 | 2680 | C | C | T | noncoding | n/a | n/a | n/a | n/a |
| 63 | 7506368 | 3787129H1 | SNP00055076 | 196 | 2609 | A | A | C | noncoding | n/d | n/a | n/a | n/a |
| 63 | 7506368 | 3991349H1 | SNP00007115 | 173 | 306 | A | C | A | K52 | 0.40 | n/a | n/a | n/a |
| 63 | 7506368 | 4054452H1 | SNP00007114 | 52 | 106 | G | T | G | noncoding | 0.21 | n/a | n/a | n/a |
| 63 | 7506368 | 4054452H1 | SNP00007115 | 251 | 306 | A | C | A | K52 | 0.40 | n/a | n/a | n/a |
| 63 | 7506368 | 4125460H1 | SNP00007114 | 50 | 106 | T | T | G | noncoding | 0.21 | n/a | n/a | n/a |
| 63 | 7506368 | 4125460H1 | SNP00007115 | 250 | 306 | A | C | A | K52 | 0.40 | n/a | n/a | n/a |
| 63 | 7506368 | 4145785H1 | SNP00007141 | 193 | 2110 | C | C | G | noncoding | 0.41 | n/a | n/a | n/a |
| 63 | 7506368 | 4152721H1 | SNP00007114 | 77 | 106 | G | T | G | noncoding | 0.21 | n/a | n/a | n/a |
| 63 | 7506368 | 4198254H1 | SNP00007115 | 177 | 306 | A | C | A | K52 | 0.40 | n/a | n/a | n/a |
| 63 | 7506368 | 4337020H1 | SNP00007115 | 128 | 306 | C | C | A | N52 | 0.40 | n/a | n/a | n/a |
| 63 | 7506368 | 4666011H1 | SNP00007115 | 132 | 306 | A | C | A | K52 | 0.40 | n/a | n/a | n/a |
| 63 | 7506368 | 483252H1 | SNP00007115 | 170 | 306 | C | C | A | N52 | 0.40 | n/a | n/a | n/a |
| 63 | 7506368 | 4846937H1 | SNP00007115 | 198 | 306 | A | C | A | K52 | 0.40 | n/a | n/a | n/a |
| 63 | 7506368 | 4919862H1 | SNP00007141 | 159 | 2110 | G | C | G | noncoding | 0.41 | n/a | n/a | n/a |
| 63 | 7506368 | 4970743H1 | SNP00007115 | 148 | 306 | A | C | A | K52 | 0.40 | n/a | n/a | n/a |
| 63 | 7506368 | 4979309H1 | SNP00007116 | 159 | 660 | C | T | C | C170 | 0.50 | n/a | n/a | n/a |
| 63 | 7506368 | 5159806H1 | SNP00007115 | 185 | 306 | A | C | A | K52 | 0.40 | n/a | n/a | n/a |
| 63 | 7506368 | 5217829H1 | SNP00007141 | 44 | 2110 | G | C | G | noncoding | 0.41 | n/a | n/a | n/a |
| 63 | 7506368 | 5394448H1 | SNP00007141 | 95 | 2110 | G | C | G | noncoding | 0.41 | n/a | n/a | n/a |
| 63 | 7506368 | 5684461H1 | SNP00007115 | 53 | 306 | C | C | A | N52 | 0.40 | n/a | n/a | n/a |
| 63 | 7506368 | 5764117H1 | SNP00007141 | 147 | 2110 | G | C | G | noncoding | 0.41 | n/a | n/a | n/a |
| 63 | 7506368 | 5782012H1 | SNP00055076 | 219 | 2609 | A | A | C | noncoding | n/d | n/a | n/a | n/a |
| 63 | 7506368 | 5833371H1 | SNP00007142 | 196 | 2680 | T | C | T | noncoding | n/a | n/a | n/a | n/a |
| 63 | 7506368 | 5833371H1 | SNP00055076 | 125 | 2609 | A | A | C | noncoding | n/d | n/a | n/a | n/a |
| 63 | 7506368 | 5976852H1 | SNP00007142 | 530 | 2680 | C | C | T | noncoding | n/a | n/a | n/a | n/a |
| 63 | 7506368 | 5976852H1 | SNP00055076 | 459 | 2609 | A | A | C | noncoding | n/d | n/a | n/a | n/a |
| 63 | 7506368 | 6152756H1 | SNP00007115 | 178 | 306 | A | C | A | K52 | 0.40 | n/a | n/a | n/a |
| 63 | 7506368 | 6369838H1 | SNP00057390 | 179 | 937 | C | A | C | Q263 | n/a | n/a | n/a | n/a |
| 63 | 7506368 | 6481283H1 | SNP00007142 | 202 | 2680 | C | C | T | noncoding | n/a | n/a | n/a | n/a |
| 63 | 7506368 | 6481283H1 | SNP00055076 | 273 | 2609 | C | A | C | noncoding | n/d | n/a | n/a | n/a |
| 63 | 7506368 | 6486589H1 | SNP00057390 | 150 | 937 | A | A | C | K263 | n/a | n/a | n/a | n/a |
| 63 | 7506368 | 6542778H1 | SNP00007115 | 198 | 306 | A | C | A | K52 | 0.40 | n/a | n/a | n/a |
| 63 | 7506368 | 6837376H1 | SNP00007141 | 7 | 2110 | C | C | G | noncoding | 0.41 | n/a | n/a | n/a |
| 63 | 7506368 | 6845404H1 | SNP00007141 | 225 | 2110 | C | C | G | noncoding | 0.41 | n/a | n/a | n/a |
| 63 | 7506368 | 6995446H1 | SNP00055076 | 405 | 2609 | C | A | C | noncoding | n/d | n/a | n/a | n/a |
| 63 | 7506368 | 7009565H1 | SNP00007141 | 276 | 2110 | G | C | G | noncoding | 0.41 | n/a | n/a | n/a |
| 63 | 7506368 | 7019695H1 | SNP00007114 | 23 | 106 | T | T | G | noncoding | 0.21 | n/a | n/a | n/a |
| 63 | 7506368 | 7019695H1 | SNP00007115 | 224 | 306 | A | C | A | K52 | 0.40 | n/a | n/a | n/a |
| 63 | 7506368 | 7019695H1 | SNP00007116 | 578 | 660 | T | T | C | C170 | 0.50 | n/a | n/a | n/a |
| 63 | 7506368 | 7067650H1 | SNP00007141 | 141 | 2110 | C | C | G | noncoding | 0.41 | n/a | n/a | n/a |
| 63 | 7506368 | 7069744H1 | SNP00007114 | 80 | 106 | G | T | G | noncoding | 0.21 | n/a | n/a | n/a |
| 63 | 7506368 | 7069744H1 | SNP00007115 | 280 | 306 | A | C | A | K52 | 0.40 | n/a | n/a | n/a |
| 63 | 7506368 | 7241663H1 | SNP00007141 | 225 | 2110 | C | C | G | noncoding | 0.41 | n/a | n/a | n/a |
| 63 | 7506368 | 7344566H1 | SNP00007115 | 207 | 306 | A | C | A | K52 | 0.40 | n/a | n/a | n/a |
| 63 | 7506368 | 753980H1 | SNP00007141 | 67 | 2110 | G | C | G | noncoding | 0.41 | n/a | n/a | n/a |
| 63 | 7506368 | 889680H1 | SNP00007142 | 37 | 2680 | C | C | T | noncoding | n/a | n/a | n/a | n/a |
| 63 | 7506368 | 998412H1 | SNP00055076 | 221 | 2609 | A | A | C | noncoding | n/d | n/a | n/a | n/a |
| 63 | 7506368 | 998775H1 | SNP00007114 | 68 | 106 | G | T | G | noncoding | 0.21 | n/a | n/a | n/a |
| 64 | 7509087 | 509134H1 | SNP00131021 | 163 | 905 | C | C | T | noncoding | n/a | n/a | n/a | n/a |

What is claimed is:

1. An isolated polypeptide selected from the group consisting of:
a) a polypeptide comprising an amino acid sequence selected from the group consisting of SEQ ID NO:1-32,
b) a polypeptide comprising a naturally occurring amino acid sequence at least 90% identical to an amino acid sequence selected from the group consisting of SEQ ID NO:1, SEQ ID NO:3-11, SEQ ID NO:13-15, SEQ ID NO:17, SEQ ID NO:19-23, SEQ ID NO:27-28, SEQ ID NO:30, and SEQ ID NO:32,
c) a polypeptide comprising a naturally occurring amino acid sequence at least 93% identical to the amino acid sequence of SEQ ID NO:2,
d) a polypeptide comprising a naturally occurring amino acid sequence at least 99% identical to an amino acid sequence selected from the group consisting of SEQ ID NO:12 and SEQ ID NO:16,
e) a polypeptide comprising a naturally occurring amino acid sequence at least 91% identical to the amino acid sequence of SEQ ID NO:24,
f) a polypeptide comprising a naturally occurring amino acid sequence at least 98% identical to an amino acid sequence selected from the group consisting of SEQ ID NO:25 and SEQ ID NO:29,
g) a polypeptide consisting essentially of a naturally occurring amino acid sequence at least 90% identical to the amino acid sequence of SEQ ID NO:31,
h) a biologically active fragment of a polypeptide having an amino acid sequence selected from the group consisting of SEQ ID NO:1-32, and
i) an immunogenic fragment of a polypeptide having an amino acid sequence selected from the group consisting of SEQ ID NO:1-32.

2. An isolated polypeptide of claim 1 comprising an amino acid sequence selected from the group consisting of SEQ ID NO:1-32.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:33-64.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-32.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:33-64,
b) a polynucleotide comprising a naturally occurring polynucleotide sequence at least 90% identical to a polynucleotide sequence selected from the group consisting of SEQ ID NO:33-62 and SEQ ID NO:64,
c) a polynucleotide consisting essentially of a naturally occurring polynucleotide sequence at least 90% identical to the polynucleotide sequence of SEQ ID NO:63,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-32.

19. A method for treating a disease or condition associated with decreased expression of functional SECP, comprising administering to a patient in need of such treatment the composition of claim 17.

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional SECP, comprising administering to a patient in need of such treatment a composition of claim 21.

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional SECP, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A diagnostic test for a condition or disease associated with the expression of SECP in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of SECP in a subject, comprising administering to said subject an effective amount of the composition of claim 32.

34. A composition of claim 32, wherein the antibody is labeled.

35. A method of diagnosing a condition or disease associated with the expression of SECP in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of an amino acid sequence selected from the group consisting of SEQ ID NO:1-32, or an immunogenic fragment thereof, under conditions to elicit an antibody response,
b) isolating antibodies from said animal, and
c) screening the isolated antibodies with the polypeptide, thereby identifying a polyclonal antibody which specifically binds to a polypeptide comprising an amino acid sequence selected from the group consisting of SEQ ID NO:1-32.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of an amino acid sequence selected from the group consisting of SEQ ID NO:1-32, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which specifically binds to a polypeptide comprising an amino acid sequence selected from the group consisting of SEQ ID NO:1-32.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide comprising an amino acid sequence selected from the group consisting of SEQ ED NO: 1-32 in a sample, the method comprising:
a) incubating the antibody of claim 11 with a sample under conditions to allow specific binding of the antibody and the polypeptide, and
b) detecting specific binding, wherein specific binding indicates the presence of a polypeptide comprising an amino acid sequence selected from the group consisting of SEQ ID NO:1-32 in the sample.

45. A method of purifying a polypeptide comprising an amino acid sequence selected from the group consisting of SEQ ID NO:1-32 from a sample, the method comprising:
a) incubating the antibody of claim 11 with a sample under conditions to allow specific binding of the antibody and the polypeptide, and
b) separating the antibody from the sample and obtaining the purified polypeptide comprising an amino acid sequence selected from the group consisting of SEQ ID NO:1-32.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO:1.

57. A polypeptide of claim 1, comprising the amino acid sequence of SEQ ID NO:2.

58. A polypeptide of claim 1, comprising the amino acid sequence of SEQ ID NO:3.

59. A polypeptide of claim 1, comprising the amino acid sequence of SEQ ID NO:4.

60. A polypeptide of claim 1, comprising the amino acid sequence of SEQ ID NO:5.

61. A polypeptide of claim 1, comprising the amino acid sequence of SEQ ID NO:6.

62. A polypeptide of claim 1, comprising the amino acid sequence of SEQ ID NO:7.

63. A polypeptide of claim 1, comprising the amino acid sequence of SEQ ID NO:8.

64. A polypeptide of claim 1, comprising the amino acid sequence of SEQ ID NO:9.

65. A polypeptide of claim 1, comprising the amino acid sequence of SEQ ID NO:10.

66. A polypeptide of claim 1, comprising the amino acid sequence of SEQ ID NO:11.

67. A polypeptide of claim 1, comprising the amino acid sequence of SEQ ID NO:12.

68. A polypeptide of claim 1, comprising the amino acid sequence of SEQ ID NO:13.

69. A polypeptide of claim 1, comprising the amino acid sequence of SEQ ID NO:14.

70. A polypeptide of claim 1, comprising the amino acid sequence of SEQ ID NO:15.

71. A polypeptide of claim 1, comprising the amino acid sequence of SEQ ID NO:16.

72. A polypeptide of claim 1, comprising the amino acid sequence of SEQ ID NO:17.

73. A polypeptide of claim 1, comprising the amino acid sequence of SEQ ID NO:18.

74. A polypeptide of claim 1, comprising the amino acid sequence of SEQ ID NO:19.

75. A polypeptide of claim 1, comprising the amino acid sequence of SEQ ID NO:20.

76. A polypeptide of claim 1, comprising the amino acid sequence of SEQ ID NO:21.

77. A polypeptide of claim 1, comprising the amino acid sequence of SEQ ID NO:22.

78. A polypeptide of claim 1, comprising the amino acid sequence of SEQ ID NO:23.

79. A polypeptide of claim 1, comprising the amino acid sequence of SEQ ID NO:24.

80. A polypeptide of claim 1, comprising the amino acid sequence of SEQ ID NO:25.

81. A polypeptide of claim 1, comprising the amino acid sequence of SEQ ID NO:26.

82. A polypeptide of claim 1, comprising the amino acid sequence of SEQ ID NO:27.

83. A polypeptide of claim 1, comprising the amino acid sequence of SEQ ID NO:28.

84. A polypeptide of claim 1, comprising the amino acid sequence of SEQ ID NO:29.

85. A polypeptide of claim 1, comprising the amino acid sequence of SEQ ID NO:30.

86. A polypeptide of claim 1, comprising the amino acid sequence of SEQ ID NO:31.

87. A polypeptide of claim 1, comprising the amino acid sequence of SEQ ID NO:32.

88. A polynucleotide of claim 12, comprising the polynucleotide sequence of SEQ ID NO:33.

89. A polynucleotide of claim 12, comprising the polynucleotide sequence of SEQ ID NO:34.

90. A polynucleotide of claim 12, comprising the polynucleotide sequence of SEQ ID NO:35.

91. A polynucleotide of claim 12, comprising the polynucleotide sequence of SEQ ID NO:36.

92. A polynucleotide of claim 12, comprising the polynucleotide sequence of SEQ ID NO:37.

93. A polynucleotide of claim 12, comprising the polynucleotide sequence of SEQ ID NO:38.

94. A polynucleotide of claim 12, comprising the polynucleotide sequence of SEQ ID NO:39.

95. A polynucleotide of claim 12, comprising the polynucleotide sequence of SEQ ID NO:40.

96. A polynucleotide of claim 12, comprising the polynucleotide sequence of SEQ ID NO:41.

97. A polynucleotide of claim 12, comprising the polynucleotide sequence of SEQ ID NO:42.

98. A polynucleotide of claim 12, comprising the polynucleotide sequence of SEQ ID NO:43.

99. A polynucleotide of claim 12, comprising the polynucleotide sequence of SEQ ID NO:44.

100. A polynucleotide of claim 12, comprising the polynucleotide sequence of SEQ ID NO:45.

101. A polynucleotide of claim 12, comprising the polynucleotide sequence of SEQ ID NO:46.

102. A polynucleotide of claim 12, comprising the polynucteotide sequence of SEQ ID NO:47.

103. A polynucleotide of claim 12, comprising the polynucleotide sequence of SEQ ID NO:48.

104. A polynucleotide of claim 12, comprising the polynucleotide sequence of SEQ ID NO:49.

105. A polynucleotide of claim 12, comprising the polynucleotide sequence of SEQ ID NO:50.

106. A polynucleotide of claim 12, comprising the polynucleotide sequence of SEQ ID NO:51.

107. A polynucleotide of claim 12, comprising the polynucleotide sequence of SEQ ID NO:52.

108. A polynucleotide of claim 12, comprising the polynucleotide sequence of SEQ ID NO:53.

109. A polynucleotide of claim 12, comprising the polynucleotide sequence of SEQ ID NO:54.

110. A polynucleotide of claim 12, comprising the polynucleotide sequence of SEQ ID NO:55.

111. A polynucleotide of claim 12, comprising the polynucleotide sequence of SEQ ID NO:56.

112. A polynucleotide of claim 12, comprising the polynucleotide sequence of SEQ ID NO:57.

113. A polynucleotide of claim 12, comprising the polynucleotide sequence of SEQ ID NO:58.

114. A polynucleotide of claim 12, comprising the polynucleotide sequence of SEQ ID NO:59.

115. A polynucleotide of claim 12, comprising the polynucleotide sequence of SEQ ID NO:60.

116. A polynucleotide of claim 12, comprising the polynucleotide sequence of SEQ ID NO:61.

117. A polynucleotide of claim 12, comprising the polynucleotide sequence of SEQ ID NO:62.

118. A polynucleotide of claim 12, comprising the polynucleotide sequence of SEQ ID NO:63.

119. A polynucleotide of claim 12, comprising the polynucleotide sequence of SEQ ID NO:64.
